SEQUENCE LISTING (36 sequences)

SEQ ID NO: 1 - DNA, 1131 bp, Caenorhabditis elegans
cgtttgcagcatgtcaagcggcgccgaacaacataatactaatggcaaaaagtgttatatgaataaaagatatgacgatgccgttgatcactattcgaaagccatcaaagtaaatcctctgccgaaatactatcagaacagagcaatgtgttactttcagcttaacaacctcaagatgactgaagaagactgcaaacgggcactggaactgagcccaaacgaagtgaaacctttatacttcttgggaaatgtatttctacagagcaaaaagtacagtgaagcaataagctgtctctccaaagcgctctaccataatgctgttatcacgaacgctccagatattgagaacgccctcaaacgggcacgccatcaaaagtacgaggaggaggagtcaaagcgaattgtacaagacgttgaatttcacacgtatttggagagcttgatcgaaaaggatcgtcaagaaaattctgaaaacccagaggagttgcaaagagcagatatggctaaaaaacgtctaaccgaactcactttagcgacacaagaaaaacggcaaaacagggaggttcctgagatgctctgcggcaaaattacactggaactgatgaaagagccggtgatagttccatctggaatcacgtatgaccgagaagaaattgtgcagcatttgagaagaattggccatttcgatccagtcacaagaaaaccacttaccgaaaatgaaattattccgaattatgcactgaaagaggttattgaaaagtttcttgacgacaacccatgggctaaatatgaacccggaggcatggtttaaatactattcagaatattgaaaaattcaacattaaacattcctggctgcttctcaatgcaacttcttcacttttctacatgtttcagttctttgcatttttttttcggatttttaaacttttattgtttgaacgctgtaccctccttttctgttctaccggtatttgtatcttggtgtattttttagctttagttttctttcaaaattccattgaatttctggtagaaagtgcacttcaacttgtcgcttagttctcaattttactgctaaaagtgagattctgtaaatattgtatgaaataaagaggtgtgatgcttttc

SEQ ID NO: 2 - protein, 266 aa, Caenorhabditis elegans
MSSGAEQHNTNGKKCYMNKRYDDAVDHYSKAIKVNPLPKYYQNRAMCYFQLNNLKMTEEDCKRALELSPNEVKPLYFLGNVFLQSKKYSEAISCLSKALYHNAVITNAPDIENALKRARHQKYEEEESKRIVQDVEFHTYLESLIEKDRQENSENPEELQRADMAKKRLTELTLATQEKRQNREVPEMLCGKITLELMKEPVIVPSGITYDREEIVQHLRRIGHFDPVTRKPLTENEIIPNYALKEVIEKFLDDNPWAKYEPGGMV

SEQ ID NO: 3 - DNA, 1226 bp, homo sapiens
ctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgacccggtgaccgggagccccctgacccaggaacagttcatccccaacttggctatgaaggaggttattgacgcattcatctctgagaatggctgggtggaggactactgaggttccctgccctacctggcgtcctggtccaggggagccctgggcagaagcccccggcccctaaacatagtttatgtttttggccaccccgaccgcttcccccaagttctgctgttggactctggactgtttcccctctcagcatcgcttttgctgggccgtgattgtcccctttgtgggctggaaaagcaggtgagggtgggctgggctgaggccattgccgccactatctgtgtaataaaatccgtgagcacgaaa

SEQ ID NO: 4 - protein, 303 aa, homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 5 - DNA, 1491 bp, Danio rerio
accgaaacatgatgctagcgtctcacacccgcgggtaaaatgtcgtgtttttgaggatcgccgatgaaaacgtcggacatttgtcatcttaatgtcatgatcgagtcggaatatgagtttggccgcgtttcattcgctatcagggatccaaaagtgatcatcaaagcaccagtgtcgctaccggtttgctaacgacaggcgtcagtccagcagtcaaacccgaggtttgcaggtaactggctccccgggaccggccggtgctcaccgaagagacggggaacaggattgggaagcaggggggagcggccaccgcagagcccatggagaagatggcgagcagcccagagaaaagctcatccgcacaggagctgaaggagcagggaaaccgcctgttcctcagccgcaagtaccaggaggccgtcacctgctacagcaaagccatcaatcgtaacccatcagtggccgtgtactacactaacagagcgctatgctacgtcaagctacagcagtacgacaaggcactggccgactgtaaacacgcccttgagcttgacagccaatcggtgaaggcgcacttttttctgggccaatgtcagctggagctggagaattacgaagaagccatcggcaacctacagagagcctataacctggcgaaggaacagaggctcaattttggagacgacatcccaagtgctcttcgcatcgccaagaagaaacgctggaacagcatagaggagaagcggatcagccaagagaacgagctgcacgcctatctcagcaaactcatcctggccgagaaggagagagagctggacgaccgagtgaagcagtcagatgacagtcagaatggaggagacatcagcaagatgaagtccaagcatgataagtatctgatggacatggatgagcttttctctcaagtagatgaaaagaggaagaagcgcgagatccccgattacctgtgtgggaagatcagcttcgagctgatgagggaaccctgcattactcccagcggcatcacctacgaccgcaaggacatcgaggagcatctacagcgtgttggccattttgaccccgtcacgcgtagtcctctgacccaagatcagctgatccccaacctggccatgaaggaggtgatcgacgctttcatccaggagaacggctgggtggaggactactgaaaacacacacacacacacactcttcctaatcacacacaataatcactcgcctgctggtctggtacacacacacttcctctcaatctgctgcatccgatgatgatgatgatgatgctgtagtttcgcctctgaatgttaagaagcaacattattgatgacacatctccaataatcattgataaagagggaactctgtgcatgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 284 aa, Danio rerio
MASSPEKSSSAQELKEQGNRLFLSRKYQEAVTCYSKAINRNPSVAVYYTNRALCYVKLQQYDKALADCKHALELDSQSVKAHFFLGQCQLELENYEEAIGNLQRAYNLAKEQRLNFGDDIPSALRIAKKKRWNSIEEKRISQENELHAYLSKLILAEKERELDDRVKQSDDSQNGGDISKMKSKHDKYLMDMDELFSQVDEKRKKREIPDYLCGKISFELMREPCITPSGITYDRKDIEEHLQRVGHFDPVTRSPLTQDQLIPNLAMKEVIDAFIQENGWVEDY

SEQ ID NO: 7 - DNA, 1227 bp, mus musculus
ggatcgctgcgcgggctgcgagatctaggtggccgggcgcggacccaagccgtgccgccggcgccatgaagggcaaggaggaaaaggagggcggcgcgcggctgggcactggtggcggcggcacgcctgataagagcccgagtgcgcaagagctcaaggagcagggaaaccggctcttcgtgggccgcaagtacccggaggcggcggcctgctacggccgcgccatcactcggaacccacttgtggcagtgtactacactaaccgggccctgtgctatctgaagatgcagcagcctgaacaggcacttgctgactgccggcgagccctggagctggacgggcagtctgtgaaggcgcacttcttcctggggcagtgccagctggagatggagagttatgatgaggccattgccaatctgcagcgagcctatagtttggccaaggagcagcgactcaactttggggatgatattcctagtgcccttcgcattgctaagaagaagcgctggaacagtatcgaggaacggcgcatccaccaggagagtgagctgcattcatatctcaccaggctcattgctgctgagcgagagagggaactggaggagtgtcagcggaaccacgagggtcatgaagatgatggccacatccgggcccagcaggcctgcattgaggccaagcacgataaatacatggcagatatggatgagctcttctctcaggtggacgagaaaagaaagaagcgagatatccctgactacttgtgtggcaagattagctttgagctgatgcgggaaccctgcattacacccagtggtatcacctatgaccgcaaggacattgaggagcacctgcagcgtgtgggccactttgaccctgtgacccggagccctctgacccaggaacagctcatccccaatttggccatgaaggaagtcattgacgctttcatctctgagaacggctgggtagaggactattgaggccccatgtcctgcctggcaccctggcccaggaggatctggagacggaagctccagtccctgtatagtttgtgtccctgggcctgcccccatcggccctgctgatgggttctgaactgctccccttctcagcataccccttgctggaccatgagcctcccttgtcccccttctgggctggagagtgggtgagggtgggctgaggttgctgctgctgccactgtcctgtaataaagtctgtgacact

SEQ ID NO: 8 - protein, 304 aa, mus musculus
MKGKEEKEGGARLGTGGGGTPDKSPSAQELKEQGNRLFVGRKYPEAAACYGRAITRNPLVAVYYTNRALCYLKMQQPEQALADCRRALELDGQSVKAHFFLGQCQLEMESYDEAIANLQRAYSLAKEQRLNFGDDIPSALRIAKKKRWNSIEERRIHQESELHSYLTRLIAAERERELEECQRNHEGHEDDGHIRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 9 - DNA, 1265 bp, Rattus norvegicus
ggaagttccggtggcggatcgccgaccgggcggagctgatagctgcgcgggctgcgagatctaggtggccgggcgcggagcccaagccgtgccgcgcggcgccatgaagggcaaggaggaaaaggagggcggcgcgcggctgggcactggtggcggcggcagccctgataagagcccgagtgcgcaagagctcaaggagcagggaaaccggctcttcgtgggccgcaagtacccggaggcggcggcctgctacggccgcgccatcacccggaacccacttgtggcagtgtactacaccaaccgggccctgtgctatctgaagatgcagcagcctgaacaggcacttgctgactgccggagagccctggagctggatgggcagtctgtgaaggcgcacttcttcctggggcagtgccagctagagatggagagttatgatgaggccattgccaatctgcagcgagcctatagtttggccaaggagcagcgactcaactttggggatgatattcctagtgcccttcgcattgctaagaagaagcgctggaacagtatcgaggaacggcgcatccaccaggagagtgagctgcactcctatctcaccaggctcattgctgctgagcgagagagggaactggaagagtgtcagcggaaccacgagggtgatgaggatgatggccacatcagggcccagcaggcctgcattgaggccaagcacgataaatacatggcggacatgaatgagctcttctctcaggtggatgagaaaagaaagaagcgagatatccctgactacttgtgtggcaagatcagctttgagctgatgcgggaaccctgcattacacccagtggcatcacctatgaccgcaaggacattgaggagcacctgcagcgcgtgggccatttcgatcctgtgacccggagccctctgacccaggaacagctcatccccaacttagccatgaaggaagtcattgatgctttcatctctgagaatggctgggtagaggactactgataccccatgtcctgcctggcaccctggcccaggagggtctggaggcagaagctccagtccctgtatatagtttgtgtccctgggcctgcccccatcggtcctgatgatgggttctgaactgctccccttctcagcatatcccttgctgggccatgagcctcccttgtctcccttctgggctggagagctggtgagggtgggttgctgctgctgctgccactgtcctgtaataaagtctgtgagcact

SEQ ID NO: 10 - DNA, 1810 bp, Rattus norvegicus
ggccgcaggcagcttctcccgattcccgaggaagcctctcgagggtggtctgacctgcgggcctctctccttccctcaattccgccgtcctaccagcggtctagacgttcccaggctgccgcatgagcccgcgtgcgccccggtggcgcgctaggacctgggcctagtgaccggccctcggagcgccacttctccagcacggaccgctgccacgttcgcgtggcgtgggccggagggtggggcttgactgccaatgggcggagccttggtctgaatattgagggtaggtcctagttactgtagggcggggccggtagagagcgcagcggtagcttctccgggctaagcgaacccgctcaatccgcgaggctccgcctttgctcacgtgtccccatgccagccccgccccacaccggaagttccggtggcggatcgccgaccgggcggagctgatagctgcgcgggctgcgagatctaggtggccgggcgcggagcccaagccgtgccgcgcggcgccatgaagggcaaggaggaaaaggagggcggcgcgcggctgggcactggtggcggcggcagccctgataagagcccgagtgcgcaagagctcaaggagcagggaaaccggctcttcgtgggccgcaagtacccggaggcggcggcctgctacggccgcgccatcacccggaacccacttgtggcagtgtactacaccaaccgggccctgtgctatctgaagatgcagcagcctgaacaggcacttgctgactgccggagagccctggagctggatgggcagtctgtgaaggcgcacttcttcctggggcagtgccagctagagatggagagttatgatgaggccattgccaatctgcagcgagcctatagtttggccaaggagcagcgactcaactttggggatgatattcctagtgcccttcgcattgctaagaagaagcgctggaacagtatcgaggaacggcgcatccaccaggagagtgagctgcactcctatctcaccaggctcattgctgctgagcgagagagggaactggaagagtgtcagcggaaccacgagggtgatgaggatgatggccacatcagggcccagcaggcctgcattgaggccaagcacgataaatacatggcggacatgaatgagctcttctctcaggtggatgagaaaagaaagaagcgagatatccctgactacttgtgtggcaagatcagctttgagctgatgcgggaaccctgcattacacccagtggcatcacctatgaccgcaaggacattgaggagcacctgcagcgcgtgggccatttcgatcctgtgacccggagccctctgacccaggaacagctcatccccaacttagccatgaaggaagtcattgatgctttcatctctgagaatggctgggtagaggactactgataccccatgtcctgcctggcaccctggcccaggagggtctggaggcagaagctccagtccctgtatatagtttgtgtccctgggcctgcccccatcggtcctgatgatgggttctgaactgctccccttctcagcatatcccttgctgggccatgagcctcccttgtctcccttctgggctggagagctggtgagggtgggttgctgctgctgctgccactgtcctgtaataaagtctgtgagcactacattggcatgtgctggtgcagtgggcttgccagtcgcttgttggctagccaaggaaagtggatatgaagacactggtgtccagattgagtgtggcatgccaccaccgatcaggaaagtacagcgcctggg

SEQ ID NO: 11 - RNA, 19 nt, homo sapiens
ggagcagggcaaucgucug

SEQ ID NO: 12 - RNA, 19 nt, homo sapiens
cagacgauugcccugcucc

SEQ ID NO: 13 - RNA, 19 nt, homo sapiens
gaucagcuuugagcugaug

SEQ ID NO: 14 - RNA, 19 nt, homo sapiens
caucagcucaaagcugauc

SEQ ID NO: 15 - RNA, 19 nt, homo sapiens
ggagguuauugacgcauuc

SEQ ID NO: 16 - RNA, 19 nt, homo sapiens
gaaugcgucaauaaccucc

SEQ ID NO: 17 - RNA, 19 nt, mus musculus
ggagcagggaaaccggcuc

SEQ ID NO: 18 - RNA, 19 nt, mus musculus
gagccgguuucccugcucc

SEQ ID NO: 19 - RNA, 19 nt, mus musculus
cuuguguggcaagauuagc

SEQ ID NO: 20 - RNA, 19 nt, mus musculus
gcuaaucuugccacacaag

SEQ ID NO: 21 - RNA, 19 nt, mus musculus
gcuccagucccuguauagu

SEQ ID NO: 22 - RNA, 19 nt, mus musculus
acuauacagggacuggagc

SEQ ID NO: 23 - DNA, 20 bp, homo sapiens
agcagggcaatcgtctgttc

SEQ ID NO: 24 - DNA, 21 bp, homo sapiens
catcttcaggtagcacaaggc

SEQ ID NO: 25 - DNA, 20 bp, homo sapiens
tgcactcctacctctccagg

SEQ ID NO: 26 - DNA, 20 bp, homo sapiens
ttgtcgtgcttggcctcaat

SEQ ID NO: 27 - DNA, 19 bp, homo sapiens
gcggacatggacgagcttt

SEQ ID NO: 28 - DNA, 20 bp, homo sapiens
ccttgcggtcgtaggtgatg

SEQ ID NO: 29 - DNA, 19 bp, mus musculus
cggcagccctgataagagc

SEQ ID NO: 30 - DNA, 21 bp, mus musculus
cacaagtgggttccgagtgat

SEQ ID NO: 31 - DNA, 21 bp, mus musculus
ccacttgtggcagtgtactac

SEQ ID NO: 32 - DNA, 22 bp, mus musculus
tggcctcatcataactctccat

SEQ ID NO: 33 - DNA, 21 bp, mus musculus
aaggagcagcgactcaacttt

SEQ ID NO: 34 - DNA, 19 bp, mus musculus
cagcagcaatgagcctggt

SEQ ID NO: 35 - DNA, 20 bp, homo sapiens
accccagccatgtacgtagc

SEQ ID NO: 36 - DNA, 19 bp, homo sapiens
gtgtgggtgaccccgtctc

(19) United States
(12) Patent Application Publication
(10) Pub. No.: US 2009/0053196 A1
(43) Pub. Date: Feb. 26, 2009

(54) Chn-1/Chip-Antagonists For The Treatment Of Muscular Diseases

(75) Inventors: Ralf Baumeister, Freiburg (DE); Thorsten Hoppe, Hamburg (DE); Britta Spanier, Freising (DE); Hanns Lochmuller, Unterhaching (DE); Rolf Stucka, Seefeld (DE)

Correspondence Address: FOLEY AND LARDNER LLP;SUITE 500, 3000 K STREET NW, WASHINGTON, DC, 20007, US

(73) Assignee: LUDWIG-MAXIMILLIANS-UNIVERSITAT MUNCHEN, MUNCHEN (DE)

(21) Appl. No.: 11/632,186

(22) PCT Filed: Jul. 29, 2005
(86) PCT No.: PCT/EP05/08273

§ 371 Date: Aug. 27, 2007

(30) Foreign Application Priority Data
Jul. 30, 2004 (EP) 04018128.1

(51) Int. Cl. A61K 31/70; A01K 67/00; A61K 38/00; A61K 39/395; C12Q 1/20

(52) U.S. Cl. 424/130.1; 435/29; 514/12; 514/44; 800/9

(57) ABSTRACT

The present invention relates to a pharmaceutical composition comprising an inhibitor/negative regulator/antagonist of the mammalian ortholog of Caenorhabditis elegans CHN-1 and/or of the human CHIP (carboxyl-terminus of Hsc70 interacting protein). Furthermore, the use of said inhibitor/negative regulator/antagonist in medical and pharmaceutical settings is described. In addition, screening methods and tools for the identification of CHIP/CHN-1 inhibitors/antagonists are provided.

The present invention relates to a pharmaceutical composition comprising an inhibitor/negative regulator/antagonist of the mammalian ortholog of Caenorhabditis elegans CHN-1 and/or of the human CHIP (carboxyl-terminus of Hsc70 interacting protein). Furthermore, the use of an inhibitor/negative regulator/antagonist of the mammalian ortholog of Caenorhabditis elegans CHN-1 and/or of the human CHIP in medical and pharmaceutical settings is described. In addition, screening methods and tools for the identification of CHIP/CHN-1 inhibitors/antagonists are provided.

Acquired muscle (myo)pathies are disorders wherein muscle fibres are affected, for example by acute inflammation and fibre necrosis (e.g. polymyositis), by complex immune and metabolic disorders, for example myasthenia gravis, myotonias or enzyme defects in the glycolytic pathways or by chronic degeneration of muscle fibres, in particular muscular dystrophies, like Duchenne muscular dystrophy or limb-girdle dystrophy.

Muscle fibres are single cells formed during development by the fusion of many separate cells. The bulk of the cytoplasm is made up of myofibrils, which are the contractile units (the sarcomeres), comprising in particular myosin and actin filaments.

Organization of the motor protein myosin into structures that perform essential processes such as cell division, cell motility, vesicular traffic and muscle development is the result of a regulated multi-step assembly pathway. Ubiquitin-dependent protein degradation is known to occur prominently in muscle cells, both in physiologic and disease states, such as during muscle atrophy.

Protein degradation by the ubiquitin/proteasome system plays an important role for cellular regulation. Substrates modified by multiubiquitin chains are usually marked for proteolysis by the 26S proteasome, a multicatalytic protease complex. Ubiquitylation of proteins requires a cascade of enzymes. Ubiquitin-activating enzyme, E1, hydrolyzes ATP and forms a high-energy thioester bond between an internal cysteine residue and the C-terminus of ubiquitin. Activated ubiquitin is then passed on to ubiquitin-conjugating enzymes, E2s, which form similar thioester-linked complexes with ubiquitin. Finally, ubiquitin is covalently attached to the substrate protein by ubiquitin-protein ligases, E3s, which often interact with the substrate directly (for recent reviews see Hershko and Ciechanover, 1998; Hochstrasser, 1996; Pickart, 2004).

Recently, the yeast protein UFD2 has been described as an additional conjugation factor, E4, which binds to the ubiquitin moieties of preformed conjugates and catalyzes multiubiquitin chain assembly in conjunction with E1, E2, and E3. E4-mediated multiubiquitylation is needed for proteasomal targeting and subsequent proteolysis of specific model substrates (Koegl, 1999). The human CHIP protein (Carboxyl-terminus of Hsc70 interacting protein) also displays E4 function, since it positively regulates the ubiquitylation activity of the E3 enzyme Parkin (Imai, 2002). The multiubiquitylation activities of these E4 enzymes may serve as an accessory option to regulate proteolysis by ubiquitin chain elongation.

Both CHIP and UFD2 contain a U-box at the C-terminus (Aravind, 2000; Hatakeyama, 2001; Koegl, 1999). In addition to its C-terminal U-box, CHIP contains three tandem tetratricopeptide repeat (TPR) motifs at the N-terminus. These TPR motifs bind to the chaperones Hsp70 and Hsp90, thereby mediating the co-chaperone activity of CHIP. Thus, CHIP provides a direct link between the chaperone and proteasome systems, and is postulated to assist in regulating the cellular balance between folding and degradation. This hypothesis is supported by the observation that in vivo overexpressed CHIP is found in activation- and folding-competent chaperone complexes with glucocorticoid receptor (GR), cystic fibrosis transmembrane conductance regulator (CFTR) and ErbB2, leading to ubiquitylation of these substrates and acceleration of their degradation through the 26S proteasome. Furthermore, heat-denatured polypeptides might also be substrates for CHIP-dependent ubiquitylation (for recent reviews see Cyr, 2002; Hohfeld, 2001; Murata, 2003).

The organization of the motor protein myosin into motile cellular structures requires precise temporal and spatial control. Myosin complexes are essential for a variety of processes such as cell division, cell motility, vesicle transport, and muscle contraction (reviewed by Barral, 1999). Members of a newly identified family of proteins containing a UCS (UNC-45/CRO1/She4p) domain are necessary for proper myosin function (reviewed by Hutagalung, 2002). They are involved in contractile ring and muscle thick filament assembly, endocytosis and mRNA localization. Mutations in C. elegans UNC-45 (Epstein, 1974), the founding member of this family, result in paralyzed animals (uncoordinated or unc phenotype) with reduced thick filament accumulation and severe myofibril disorganization (Barral, 1998). The UNC-45 protein exerts chaperone activity on the myosin head through its C-terminal UCS domain, and functions as an Hsp90 co-chaperone through its N-terminal TPR domain (Barral, 2002). UNC-45 homologs have been identified in a variety of organisms, including Drosophila, Xenopus, zebrafish, mouse, and human (Hutagalung, 2002).

The main factor causing dystrophies, in particular Duchenne muscle dystrophy (DMD) is the degradation of muscle proteins normally forming the dystrophin-glycoprotein complex (DGC), that connects the actin cytoskeleton with the extracellular matrix and stabilize the muscle structure. Loss of dystrophin induces un- and misfolding of other DGC proteins, which are deactivated and degradated over the ubiquitin-proteasome pathway (Passmore, 2004). In eukaryotic cells, most un- and misfolded proteins are degradated by the ubiquitin-proteasome system. E1 enzymes activate the ubiquitin and transfer it to E2 enzymes, that interact with E3 ubiquitin ligases, which catalyse the conjugation of several ubiquitin molecules with the targeted protein. The regulation of the ubiquitylation is enabled by the highly specific substrate detection and binding of the E3 ubiquitin ligases. The polyubiquitin chain is the signal for proteolysis of marked proteins at the 26S proteasome. Bonuccelli (2003) was able to rescue the expression and localisation of dystrophin and DGC proteins by blockade of the ubiquitin-proteasome system with the proteasome inhibitor MG-132 in dystrophin knockout mice (mdx mice).

Muscular dystrophies are progressive, genetically determined disorders of skeletal and sometimes cardiac muscle. Duchenne muscular dystrophy (DMD) is inherited as an X-linked recessive disorder, but one-third of cases arise by spontaneous mutations. It occurs in 1 in 3000 male infants. Recently the DMD locus has been localized to the Xp21 region of the X chromosome and the disease is characterized by the absence of the gene product—the protein dystrophin, which is a rod-shaped cytoskeletal protein found in muscle. DMD is usually obvious by the fourth or sixth year, and causes death by the age of 20 years or, under specific treatment, by the age of 40 years.

The clinical features of this disease are that the boy has difficulty in running and in rising to an erect position, when he has to “climb up his legs with his hands” (Gowers' sign). There is initially a proximal limb weakness with pseudohypertrophy of the calves. The myocardium is affected. The boy becomes severely disabled by 10 years.

The diagnosis is often made on clinical grounds alone. The creatine phosphokinase is grossly elevated (100-200 times the normal level). Muscle biopsy shows characteristic variation in fibre size, fibre necrosis, regeneration and replacement by fat, and on immunochemical staining an absence of dystrophin. The electromyogram shows a myopathic pattern.

There is no curative treatment. Passive physiotherapy helps to prevent contractures in the later stages of the disease. A trial of prednisolone therapy has shown a short term improvement in muscle strength and function.

A female with an affected brother has a 50% chance of carrying the gene. In carrier females, 70% have a raised creatine phosphokinase level and the remainder usually have electromyographic abnormalities or changes on biopsy. Accurate carrier and prenatal diagnosis can be made using cDNA probes that are co-inherited with the DMD locus.

Genetic advice explaining the inheritance of the condition and counselling about abortion should be given. Determination of the fetal sex by amniocentesis and selective abortion of a male fetus is sometimes carried out. Many proven carrier females choose not to have offspring.

The technical problem underlying the present invention is the provision of means and method for the treatment, amelioration and/or prevention of muscle (myo)pathies, in particular of muscular dystrophies.

The solution to this technical problem is achieved by providing the embodiments characterized in the specification and the claims.

The present invention provides for a pharmaceutical composition comprising an inhibitor/negative regulator of the mammalian ortholog of Caenorhabditis elegans CHN-1 and/or of the human CHIP (carboxyl-terminus of Hsc70 interacting protein).

Accordingly, the invention provides for antagonists of CHIP/CHN-1 expression, function or activity in medical settings. In accordance with this invention the term “CHN-1” relates to the C. elegans ortholog of human “CHIP”—whereby CHIP is the carboxyl-terminus of Hsc70 interacting protein—as described in the art, e.g. Immai, 2002; Hatakeyama, 2001 or Koegl, 1999.

Furthermore, the invention envisages that inhibitor/antagonists and/or negative regulators of CHN-1, CHIP and/or orthologs or homologs of CHN-1/CHIP are employed in the herein disclosed pharmaceutical compositions, uses and methods.

Orthologs as well as homologs of CHN-1/CHIP are known in the art and comprise, inter alia, the amino acid sequences as encoded by the nucleic acid molecules depicted in SEQ ID NOs: 1, 3, 5, 7, 9, 11 and 12 or the amino acid sequence as shown in SEQ ID NOs: 2, 4, 6, 8, 10, whereby SEQ ID NOs 1 and 2 relate to C. elegans sequences, whereby SEQ ID NOs 3 (as well as 11) and 4 relate to human sequences, whereby SEQ ID NOs 5 and 6 relate to zebrafish sequences, whereby SEQ ID NOs 7 and 8 relate to mouse sequences, whereby SEQ ID NOs 9 (as well as 12) and 10 relate to rat sequences.

The term “ortholog” as employed herein relates to genes and gene products in different species that retain the same function. Accordingly “CHIP” is the human ortholog of C. elegans CHN-1 and vice versa. The function of a CHIPortholog or a CHN-1 ortholog in context of this invention is at least the ubiquitylation/multi-ubiquitylation of UNC-45. Another function of a CHIP/CHN-1 ortholog is the interaction with Parkin. Also envisaged in context of this invention is the pharmaceutical and medical use of homologous of CHIP/CHN-1 as defined herein. Homologies are defined below.

As documented in the appended examples, it was surprisingly found that UFR-2 and CHN-1, the C. elegans homologues of human UFD2 and human CHIP, act in concert to multiubiquizylate the myosin chaperone UNC-45, thus revealing that selective protein degradation contributes to proper myosin assembly. A particular unexpected key finding was that UFD-2 and CHN-1 interact with UNC-45. UNC-45 is a member of the UCS (UNC-45/CRO1/She4p) family of proteins that are required for myosin assembly. UNC-45 helps ensure that muscle myosin is properly folded through a dual mechanism: by exerting chaperone activity directly on the myosin head and by acting as a co-chaperone for the heat shock protein Hsp90. Furthermore, it was found that UNC-45 is a substrate for UFD-2- or CHN-1-dependent ubiquitylation. Unexpectedly, most conjugated substrates contained only one to three ubiquitin moieties, indicating that C. elegans UFD-2 and CHN-1 merely act as E3 ubiquitin ligases on their own. Strikingly however, simultaneous addition of UFD-2 and CHN-1 dramatically enhanced multiubiquitylation of UNC-45. These findings document that E4 activity is achieved by the formation of a heterooligomeric complex comprised of two E3 enzymes.

A specific E4 multiubiquitylation activity is described herein, whereas said activity is formed by UFD-2 and the CHIP ortholog CHN-1 in Caenorhabditis elegans. Surprisingly, this E3/E4 complex is necessary and sufficient to multiubiquitylate the myosin chaperone UNC-45 in vitro. In vivo, the myosin assembly defects of unc-45 temperature-sensitive animals are partially suppressed by chn-1 loss-of-function, while UNC-45 overexpression in worms deleted for chn-1 results in severely disorganized muscle cells. Accordingly, and as demonstrated in the examples, CHN-1 and UFD-2 form a functional E3/E4 complex that regulates UNC-45 levels, thereby permitting proper assembly of myosin into muscle thick filaments.

Furthermore, the regulatory interaction between the C. elegans orthologs of the E4 enzymes CHIP and UFD2, CHN-1 and UFD-2, respectively is described. This interaction is required for proper UNC-45-directed myosin assembly. CHN-1 and UFD-2 in conjunction with the E2 enzyme LET-70 drive multiubiquitylation of UNC-45 in vitro. In the absence of CHN-1 or UFD-2 only a few ubiquitin molecules are ligated to UNC-45. Unlike most CHIP-dependent substrates, UNC-45 ubiquitylation does not depend on the general chaperone activities of Hsp70 or Hsp90. Accordingly, CHN-1 and UFD-2 form a functional E3/E4 complex that is necessary to multiubiquitylate the myosin chaperone UNC-45, thereby regulating myosin folding and consequently, assembly.

The findings presented in the context of this invention show that CHN-1 and its human homolog/ortholog CHIP is a negative regulator of UNC-45 in muscle cells. Without being bound by theory, this is related to CHIPs ability to promote, in concert with UFD-2, multiubiquitylation and proteasome-mediated degradation of UNC-45. These results show that multiubiquitylation is crucial for proper myosin assembly by setting the levels of UNC-45 available for aiding myosin folding.

The ubiquitin-proteasome pathway is essential for the degradation of many, if not most unfolded, misfolded or superfluous proteins. However, a multitude of enzymes (E1, E2, E3, E4) are involved in the initial step of protein degradation, the ubiquitylation of protein. These enzymes have mostly very specific substrates. This is particularly true for the E3 ubiquitin ligases. As a consequence, only for very few E3 ubiquitin ligases a substrate is known. Therefore, although a blockade of the entire proteasome system will interfere with many, if not most diseases with defects in protein folding and stability, the effect is not specific and the interference in many cases is even detrimental for the organism, in light of this non-specificity. The present invention provides a substrate for the E3 ligase CHIP. Since this substrate is involved in assembly of the muscular myosin thick filaments, it could be shown in the appended examples that (temperature-sensitive, weak) mutants with defects in muscular thick filament assembly that are preferably degraded by the ubiquitin-proteasome pathway can be suppressed by reducing/eliminating CHIP activity. Muscular Dystrophies, in particular Duchenne Muscular Dystrophy (DMD) are caused, inter alia, by mutations in the dystrophin gene. Phenotypically, these diseases resemble the muscular dystrophies caused by C. elegans unc-45 mutants, whereby the UNC-45 is the CHIP substrate identified herein. Reducing the activity of CHN-1/CHIP or CHN-1/CHIP orthologs has a beneficial effect on DMD mutants. In consequence, two different types of mutants resulting in dystrophic musculature can be suppressed by reducing CHIP/CHN-1 gene activity, as documented in the examples.

The findings of the present invention are of particular interest in the therapy of mammalian, and in particular of human, muscular disorders, preferably of dystrophies and most preferably of Duchenne muscular dystrophy.

The human homolog (or ortholog) of CHN1, CHIP (Ballinger, 1999) (also known as STUB1) can on its own exert E4 activity, however, in C. elegans the formation of an UFD-2/CHN-1 heterooligomer is essential for UNC-45 ubiquitylation/multiubiquitylation. In context of this invention it is envisaged that CHIP, preferably a CHIP homooligomer, functions in the mammalian, in particular the human, system in an analogous manner to the UFD-2/CHN-1 heterooligomer. Accordingly, the present invention provides for means and method for the treatment of muscular diseases, in particular of dystrophies, by the use of an inhibitor/negative regulator/antagonist of CHIP expression, function and/or activity.

A further object of the present invention is the use of an inhibitor/negative regulator/antagonist of the mammalian ortholog of C. elegans CHN-1 and/or of human CHIP for the preparation of a pharmaceutical composition for the treatment, amelioration and/or prevention of a myopathy or a muscular disease. Accordingly, also provided is a method for treating, ameliorating and/or preventing a myopathy or a muscular disease in a subject comprising administering an inhibitor/negative regulator/antagonist of the mammalian ortholog of C. elegans CHN-1 and/or of the human CHIP to mammals in need of such a therapy.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

The term “inhibitor” comprises competitive, non-competitive, functional and chemical antagonists as described, inter alia, in Mutschler, “Arzneimittelwirkungen” (1986), Wissenschaftliche Verlagsgesellschaft mbH, Stuttgart, Germany. The term “partial inhibitor” in accordance with the present invention means a molecule or substance or compound or composition or agent or any combination thereof that is capable of incompletely blocking the action of agonists through, inter alia, a non-competitive mechanism. It is preferred that said inhibitor alters, modulates and/or prevents expression, activation or function of CHN-1/CHIP orthologs or the interaction of CHN-1/CHIP ortholog with its in vivo binding partner as defined herein and illustrated in the appended examples. Preferably, said inhibition leads to partial, preferably complete, standstill. Said standstill may either be reversible or irreversible.

Preferably, the inhibitor/antagonist of a CHN-1/CHIP ortholog alters, modulates and/or prevents ubiquitylation events. Further functions which may be altered, modulated or prevented comprise the interaction with UFD2, the interaction with other E3 enzymes, the interaction with E2 enzymes or the interaction with UNC-45 or fragments of UNC-45. The appended examples provide for test systems how the modulation, alteration and/or prevention of an ubiquilation event can be measured in vitro (for example in cultured cells, cell lysates or cell-free systems) and in vivo, (for example in non-human test animals, like C. elegans).

Accordingly, as documented in the appended examples, the person skilled in the art is readily in a position to screen any given compound or substance for its inhibiting activity on CHN-1/CHIP. The corresponding assays may comprise in vitro assays, cell-based assay as well as assays on and with non-human test animals. As documented in the appended examples, the inhibitory effect on CHN-1/CHIP may also be tested in cellular systems, like cultured human muscle cells.

In a preferred embodiment of the invention an inhibitor/negative regulator/antagonist to be used in the pharmaceutical composition in the preparation of a medicament or the methods of the invention is selected from the group consisting of small-binding molecules, intracellular-binding receptors, aptamers, intramers, RNAi (double-stranded RNA), siRNA and anti-CHN-1- or anti-CHIP-antisense molecules. Preferred siRNAs are shown in SEQ ID NOS: 13 to 24. Furthermore inhibitors/antagonists are mentioned herein below in context of the inventive screening methods provided herein.

The intracellular binding receptor to be used may be an intracellular antibody or an antibody fragment directed against CHN-1/CHIP and/or an ortholog of CHN-1/CHIP.

As documented in the appended examples, in particular inhibiting or interfering nucleic acid molecules, e.g. siRNA or RNAi may successfully be employed in context of this invention and may be used to modify the CHN-1/CHIP activity or function.

Also envisaged are inhibitors/antagonists/negative regulators of CHN-1/CHIP function or activity are truncated and/or mutated CHN-1/CHIP molecules (e.g. CHN-1/CHIP orthologs in other species than C. elegans and human), which interfere with or are capable of interfering with the complex formation of CHN-1/CHIP with its binding partners defined herein and described in the experimental part of the invention.

(Small) binding molecules to be employed as inhibitors/antagonists/negative regulators of CHN-1/CHIP function, activity or expression may comprise natural as well as synthetic compounds. The term “compound” in context of this invention comprises single substances or a plurality of substances. Said compound/binding molecules may be comprised in, for example, samples, e.g., cell extracts from, e.g., plants, animals or microorganisms. Furthermore, said compound(s) may be known in the art but hitherto not known to be capable of (negatively) influencing the activity of CHN-1/CHIP or CHN-1/CHIPorthologs or not known to be capable of influencing the expression of the nucleic acid molecule encoding for CHN-1/CHIP or CHN-1/CHIPorthologs, respectively. The plurality of compounds may be, e.g., added to a sample in vitro, to the culture medium or injected into the cell.

If a sample (collection of compounds) containing (a) compound(s) is identified in the art as a specific inhibitory binding molecule of CHN-1/CHIP or CHN-1/CHIP orthologs, then it is either possible to isolate the compound from the original sample identified as containing the compound in question or one can further subdivide the original sample, for example, if it consists of a plurality of different compounds, so as to reduce the number of different substances per sample and repeat the method with the subdivisions of the original sample. It can then be determined whether said sample or compound displays the desired properties, i.e. the inhibition of CHN-1/CHIP or CHN-1/CHIP orthologs, by methods known in the art. Depending on the complexity of the samples, the steps described above can be performed several times, preferably until the sample identified according to the screening method only comprises a limited number of or only one substance(s). Preferably said sample comprises substances of similar chemical and/or physical properties, and most preferably said substances are identical.

As pointed out above, binding molecules/inhibitory molecules for CHN-1/CHIP or CHN-1/CHIP orthologs may be deduced by methods in the art. Such methods comprise, e.g., but are not limited to methods, where a collection of substances is tested for interaction with CHN-1/CHIP or CHN-1/CHIP orthologs or with (a) fragment(s) thereof and where substances which test positive for interaction in a corresponding readout system are further tested in vivo, in-vitro or in silico for their inhibitory effects on CHN-1/CHIP or CHN-1/CHIP orthologs expression or function.

Said “test for CHN-1/CHIP or CHN-1/CHIP orthologs interaction” of the above described method may be carried out by specific immunological, molecular biological and/or biochemical assays which are well known in the art and which comprise, e.g., homogenous and heterogenous assays as described herein below.

Said interaction assays employing read-out systems are well known in the art and comprise, inter alia, two hybrid screenings (as, described, inter alia, in EP-0 963 376, WO 98/25947, WO 00/02911), GST-pull-down columns, co-precipitation assays from cell extracts as described, inter alia, in Kasus-Jacobi, Oncogene 19 (2000), 2052-2059, “interaction-trap” systems (as described, inter alia, in U.S. Pat. No. 6,004,746) expression cloning (e.g. lamda gtII), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271, WO 00/05410 or Yeast Four hybrid assays as described in Sandrok & Egly, JBC 276 (2001), 35328-35333.

Said interaction assays for CHN-1/CHIP or CHN-1/CHIP orthologs also comprise assays for FRET-assays, TR-FRETs (in “A homogenius time resolved fluorescence method for drug discovery” in: High throughput screening: the discovery of bioactive substances. Kolb, (1997) J. Devlin. NY, Marcel Dekker 345-360) or commercially available assays, like “Amplified Luminescent Proximity Homogenous Assay”, BioSignal Packard. Furthermore, the yeast-2-hybrid (Y2H) system may be employed to elucidate further particular and specific interaction, association partners of CHN-1/CHIP or CHN-1/CHIP orthologs. Said interaction/association partners are further screened for their inhibitory effects, in particular also their effect on ubiquitylation.

Similarly, interacting molecules (for example) (poly)peptides (for example also fragments of CHN-1/CHIP functioning as binding molecules and inhibitory molecules) may be deduced by cell-based techniques well known in the art. These assays comprise, inter alia, the expression of reporter gene constructs or “knock-in” assays, as described, for, e.g., the identification of drugs/small compounds influencing the (gene) expression of CHN-1/CHIP or CHN-1/CHIP orthologs. Said “knock-in” assays may comprise “knock-in” of CHN-1/CHIP or CHN-1/CHIP orthologs (or (a) fragment(s) thereof) in tissue culture cells, as well as in (transgenic) animals. Examples for successful “knock-ins” are known in the art (see, inter alia, Tanaka, J. Neurobiol. 41 (1999), 524-539 or Monroe, Immunity 11 (1999), 201-212). Furthermore, biochemical assays may be employed which comprise, but are not limited to, binding of the CHN-1/CHIP or CHN-1/CHIP orthologs (or (a) fragment(s) thereof) to other molecules/(poly)peptides, peptides or binding of the CHN-1/CHIP or CHN-1/CHIP orthologs (or (a) fragment(s) thereof) to itself (themselves) (dimerizations, oligomerizations, multimerizations) and assaying said interactions by, inter alia, scintillation proximity assay (SPA) or homogenous time-resolved fluorescence assay (HTRFA).

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs, FIAs, CLIAs or ECLs.

As discussed below the interaction of the inhibiting molecules of CHN-1/CHIP or CHN-1/CHIP orthologs mRNA and CHN-1/CHIP or CHN-1/CHIP orthologs protein or fragments thereof may also be tested by molecular biological methods, like two-, three- or four-hybrid-assays, RNA protection assays, Northern blots, Western blots, micro-, macro- and Protein- or antibody arrays, dot blot assays, in situ hybridization and immunohistochemistry, quantitative PCR, coprecipitation, far western blotting, phage based expression cloning, surface plasmon resonance measurements, yeast one hybrid screening, DNAse I, footprint analysis, mobility shift DNA-binding assays, gel filtration chromatography, affinity chromatography, immunoprecipitation, one- or two dimensional gel electrophoresis, aptamer technologies, as well as high throughput synthesis and screening methods.

The compounds identified and/or obtained according to the above described method(s), in particular inhibitors of CHN-1/CHIP or CHN-1/CHIP orthologs or (a) fragment(s) thereof, are expected to be very beneficial as agents in pharmaceutical settings disclosed herein and to be used for medical purposes, in particular, in the treatment of muscular disorders described herein.

Compounds which may function as specific inhibition of CHN-1/CHIP or CHN-1/CHIP orthologs also comprise (small) organic compounds, like compounds which can be used in accordance with the present invention include, inter alia, peptides, proteins, nucleic acids including cDNA expression libraries, small organic compounds, ligands, PNAs and the like. Said compounds can also be functional derivatives or analogues. Methods for the preparation of chemical derivatives and analogues are well known to those skilled in the art and are described in, for example, Beilstein, “Handbook of Organic Chemistry”, Springer Edition New York, or in “Organic Synthesis”, Wiley, New York. Furthermore, said derivatives and analogues can be tested for their effects, i.e. their inhibitory effects of CHN-1/CHIP or CHN-1/CHIP orthologs according to methods known in the art. Furthermore, peptidomimetics and/or computer aided design of appropriate inhibitors of CHN-1/CHIP or CHN-1/CHIP orthologs can be used. Appropriate computer systems for the computer aided design of, e.g., proteins and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for, e.g., optimizing known compounds, substances or molecules. Appropriate compounds can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors of CHN-1/CHIP or CHN-1/CHIP orthologs can be used for the design of (peptidomimetic) inhibitors of CHN-1/CHIP or CHN-1/CHIP orthologs (Rose, Biochemistry 35 (1996), 12933-12944; Rutenber, Bioorg. Med. Chem. 4 (1996), 1545-1558).

As mentioned herein above, the inhibitor of CHN-1/CHIP or CHN-1/CHIP orthologs expression or function may also comprise an aptamer.

In the context of the present invention, the term “aptamer” comprises nucleic acids such as RNA, ssDNA (ss=single stranded), modified RNA, modified ssDNA or PNAs which bind a plurality of target sequences having a high specificity and affinity. Aptamers are well known in the art and, inter alia, described in Famulok, Curr. Op. Chem. Biol. 2 (1998), 320-327. The preparation of aptamers is well known in the art and may involve, inter alia, the use of combinatorial RNA libraries to identify binding sites (Gold, Ann. Rev. Biochem. 64 (1995), 763-797).

Accordingly, aptamers are oligonucleotides derived from an in vitro evolution process called SELEX (systematic evolution of ligands by exponential enrichment). Pools of randomized RNA or single stranded DNA sequences are selected against certain targets. The sequences of tighter binding with the targets are isolated and amplified. The selection is repeated using the enriched pool derived from the first round selection. Several rounds of this process lead to winning sequences that are called ‘aptamers’ or ‘ligands’. Aptamers have been evolved to bind proteins which are associated with a number of disease states. Using this method, many powerful antagonists of such proteins can be found. In order for these antagonists to work in animal models of disease and in humans, it is normally necessary to modify the aptamers. First of all, sugar modifications of nucleoside triphosphates are necessary to render the resulting aptamers resistant to nucleases found in serum. Changing the 2′OH groups of ribose to 2′F or 2′NH2 groups yields aptamers which are long lived in blood. The relatively low molecular weight of aptamers (8000-12000) leads to rapid clearance from the blood. Aptamers can be kept in the circulation from hours to days by conjugating them to higher molecular weight vehicles. When modified, conjugated aptamers are injected into animals, they inhibit physiological functions known to be associated with their target proteins. Aptamers may be applied systemically in animals and humans to treat organ specific diseases (Ostendorf (2001) J. Am. Soc. Neph. 12, 909-918). The first aptamer that has proceeded to phase I clinical studies is NX-1838, an injectable angiogenesis inhibitor that can be potentially used to treat macular degeneration-induced blindness. (Sun (2000) Curr. Op. Mol. Ther. 2, 100-105). Cytoplasmatic expression of aptamers (“intramers”) may be used to inhibit intracellular targets (Mayer (2001) PNAS 98, 4961-4965). Said intramers are also envisaged to be employed in context of this invention.

Said (other) receptors of CHN-1/CHIP or CHN-1/CHIP orthologs may, for example, be derived from (an) antibody(ies) against CHN-1/CHIP or CHN-1/CHIP orthologs by peptidomimetics. The specificity of the recognition implies that other known proteins, molecules are not bound.

The RNAi-approach is also envisaged in context of this invention for use in the preparation of a pharmaceutical composition for the treatment of muscular diseases disclosed herein.

The term RNA interference (RNAi) describes the use of double-stranded RNA to target specific mRNAs for degradation, thereby silencing their expression. Double-stranded RNA (dsRNA) matching a gene sequence is synthesized in vitro and introduced into a cell. The dsRNA feeds into a natural, but only partially understood process including the highly conserved nuclease DICER (Hutvagner, 2001; Grishok, 2001), which cleaves dsRNA precursor molecules into short interfering RNAs (siRNAs). The generation and preparation of siRNA(s) as well as the method for inhibiting the expression of a target gene is, inter alia, described in WO 02/055693, Wei (2000) Dev. Biol. 15, 239-255; La Count (2000), Biochem. Paras. 111, 67-76, Baker (2000) Curr. Biol. 10, 1071-1074, Svoboda (2000), Development 127, 4147-4156 or Marie (2000) Curr. Biol. 10, 289-292. These siRNAs built then the sequence specific part of an RNA-induced silencing complex (RISC), a multicomplex nuclease that destroys messenger RNAs homologous to the silencing trigger. One protein-part of the ribonucleoprotein complex has been identified as Argonaute2 (Hammond, 2001). Elbashir (2001) showed that duplexes of 21 nucleotide RNAs may be used in cell culture to interfere with gene expression in mammalian cells.

Methods to deduce and construct siRNAs are in the art and are described in the appended examples as well as in Elbashir et al., 2002, at the internet web sites of commercial vendors of siRNA, e.g. Xeragon Inc. (www.xeragon.com/siRNA support.html); Dharmacon (www.dharmacon.com); Xeragon Inc. (www.xeragon.com), and Ambion (www.ambion.com), or at the web site of the research group of Tom Tuschl (http://www.rockefeller.edu/labheads/tuschl/index.html). In addition, programs are available online to deduce siRNAs from a given mRNA sequence (e.g. http://www.ambion.com/techlib/misc/siRNA_finder.html or http://katahdin.cshl.org:9331/RNAi/). Uridine residues in the 2-nt 3′ overhang can be replaced by 2′deoxythymidine without loss of activity, which significantly reduces costs of RNA synthesis and may also enhance resistance of siRNA duplexes when applied to mammalian cells (Elbashir, 2001). This modification may also be incorporated in specific, exemplified siRNAs provided herein (see, SEQ ID NOS: 13 to 18 for human or SEQ ID NOS: 19 to 24 for mouse) (see below) of the present application. The siRNAs may also be synthesized enzymatically using T7 or other RNA polymerases (Donze, 2002). Short RNA duplexes that mediate effective RNA interference (esiRNA) may also be produced by hydrolysis with Escherichia coli Rnase III (Yang, 2002). Furthermore, expression vectors have been developed to express double stranded siRNAs connected by small hairpin RNA loops in eukaryotic cells (e.g. (Brummelkamp, 2002)). All of these constructs may be developed with the help of the programs named above. In addition, commercially available sequence prediction tools incorporated in sequence analysis programs or sold separately, e.g. the siRNA Design Tool offered by www.oligoEngine.com (Seattle, Wash.) may be used for siRNA sequence prediction.

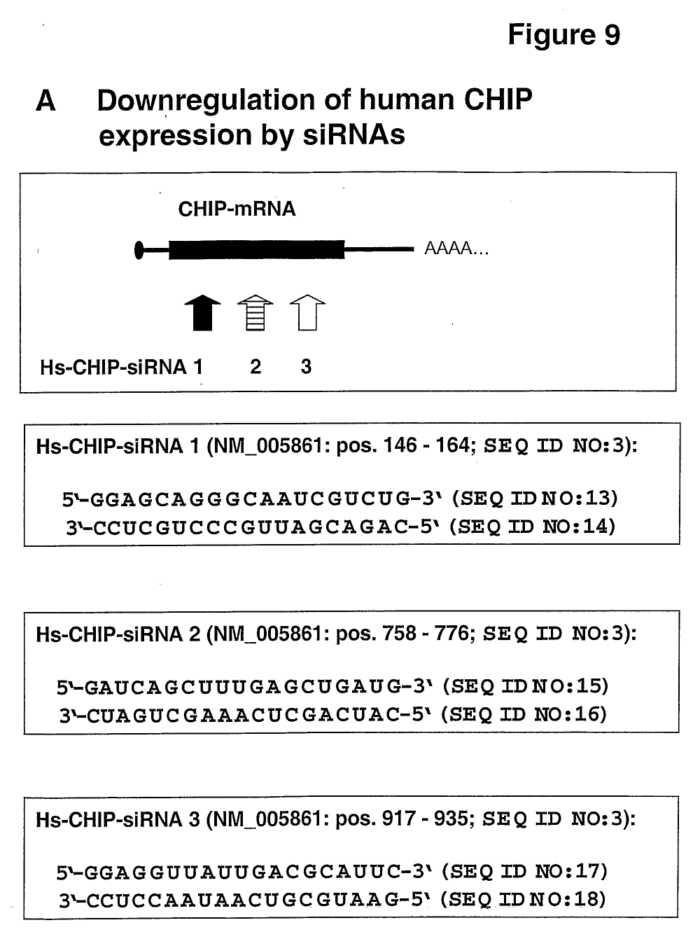

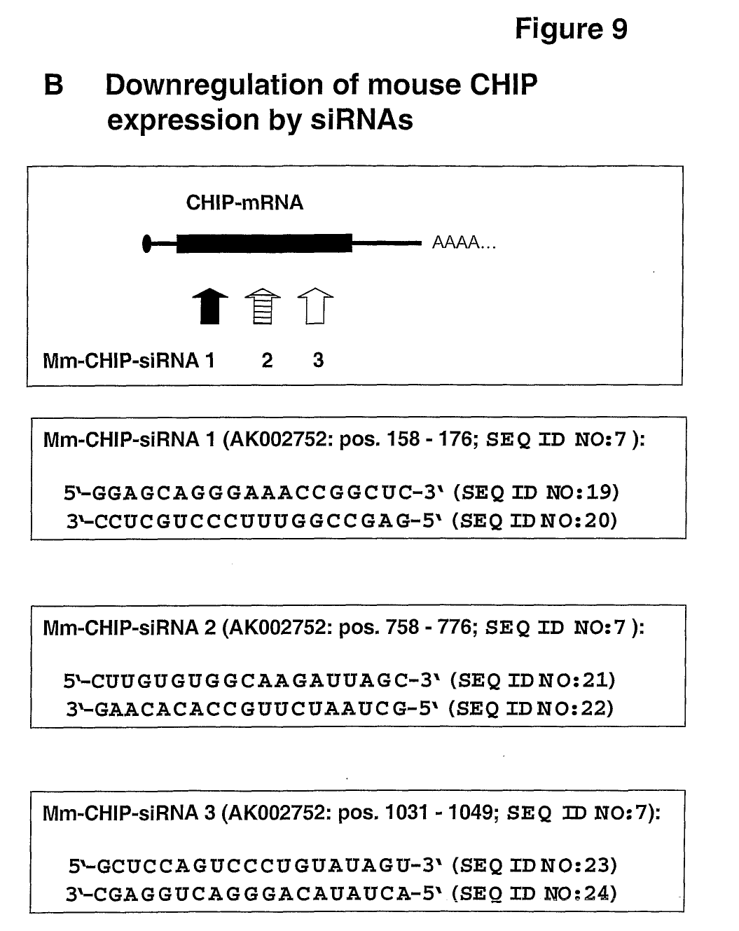

Accordingly, the present invention also provides for the use of specific interfering RNAs as inhibitors of CHN-1/CHIP or CHN-1/CHIP orthologs expression, activity and/or function. Preferably, said (small) interfering RNAs (siRNAs) comprise at least 10, more preferably at least 12, more preferably at least 14, more preferably at least 16, more preferably at least 18 nucleotides. Corresponding examples are given in appended FIG. 9 as well as in SEQ ID NOS: 13 to 24, whereby the duplexes as shown in pairs SEQ ID NOS: 13/14, 15/16 and 17/18 represent potentially useful siRNAs for downregulation of human CHIP and duplexes as shown in SEQ ID NOS: 19/20, 21/22 and 23/24 are siRNAs for the downregulation of the mouse homologue. The above recited exemplified siRNAs are not only useful in the herein described medical setting but are also valuable research tools.

For example siRNAs directed against human CHIP may be employed in experimental in vitro settings using cell culture systems. Such cell culture systems may comprise but are not limited to cultured myoblasts from patients, preferably patients suffering from Duchenne muscular dystrophy (DMD), sarcoglycanopathies as well as limb-girdle dystrophy, facio-scapulo-humeral dystrophy and the like (see below). Accordingly, these cellular systems are an example how inhibitory molecules/negative regulators/antagonists of a mammalian ortholog of CHIP-1/CHIP can be measured and tested.

Also the herein defined mouse siRNAs are useful in a pre-clinical and research setting. Here the siRNAs may be employed in cultured cells, preferably cultured mouse myoblasts as well as in an in vivo setting employing mouse models as defined below. Examples comprise but are not limited to mdx-, dy-Dag-1-mice (see table below). A corresponding experimental setting is given in the examples part. Furthermore, siRNAs directed against the cyclin-dependent kinase inhibitor p21 has previously successfully been employed in an in vitro Duchenne model; see Endesfelder (2005) J. Mol. Med. 83, 64-71.

Intracellular antibodies are known in the art and can be used to neutralize or modulate the functional activity of the target molecule. This therapeutic approach is based on intracellular expression of recombinant antibody fragments, either Fab or single chain Fv, targeted to the desired cell compartment using appropriate targeting sequences (summarized in Teillaud (1999) Pathol. Biol. 47, 771-775).

As mentioned herein above, a further preferred inhibitor of CHN-1/CHIP or CHN-1/CHIP orthologs expression and/or function is an antisense molecule. Preferably said anti-CHN-1/CHIP or CHN-1/CHIP orthologs antisense molecule comprises a nucleic acid molecule which is the complementary strand of a reversed complementary strand of the coding region of CHN-1/CHIP or CHN-1/CHIP orthologs.

Coding regions of CHN-1/CHIP or CHN-1/CHIP orthologs are known in the art and comprise, inter alia, the CHN-1/CHIP or CHN-1/CHIP orthologs GenBank entries for mouse (AK002752), for rat (XP_{—}213270) for protein; XM_{—}213270 for nucleic acid molecule, for human (NM_{—}005861), for C. elegans (NM_{—}059380) or for zebrafish (AAH51775) for protein; NM_{—}199674 for nucleic acid molecule. The person skilled in the art may easily deduce the relevant coding region of CHN-1/CHIP or CHN-1/CHIP orthologs in these GenBank entries, which may also comprise the entry of genomic DNA as well as mRNA/cDNA.

Furthermore, it is also envisaged that the antisense molecules against CHN-1/CHIP or CHN-1/CHIP orthologs expression, activity or function interfere specifically with promoter regions of CHN-1/CHIP or CHN-1/CHIP orthologs.

It is envisaged that the antisense molecules to be used in accordance with the present invention inhibit the expression or function of CHN-1/CHIP or CHN-1/CHIP orthologs, in particular of human CHN-1/CHIP or CHN-1/CHIP orthologs and interact with CHN-1/CHIP or CHN-1/CHIP orthologs as expressed by the coding regions, mRNAs/cDNAs as deposited under the above mentioned GenBank accession numbers as well as with CHN-1/CHIP or CHN-1/CHIP orthologs as expressed by isoforms and variants of said CHN-1/CHIP or CHN-1/CHIP orthologs. Said isoforms or variants may, inter alia, comprise allelic variants or splice variants.

The term “variant” means in this context that the CHN-1/CHIP or CHN-1/CHIP orthologs nucleotide sequence and the encoded CHN-1/CHIP or CHN-1/CHIP orthologs amino acid sequence, respectively, differs from the distinct sequences available under said GenBank Accession numbers, by mutations, e.g. deletion, additions, substitutions, inversions etc.

Therefore, the antisense-molecule to be employed in accordance with the present invention specifically interacts with/hybridizes to one or more nucleic acid molecules encoding CHN-1/CHIP or CHN-1/CHIP orthologs. Preferably said nucleic acid molecule is RNA, i.e. pre mRNA or mRNA. The term “specifically interacts with/hybridizes to one or more nucleic acid molecules encoding CHN-1/CHIP or CHN-1/CHIP orthologs” relates, in context of this invention, to antisense molecules which are capable of interfering with the expression of CHN-1/CHIP or CHN-1/CHIP orthologs. The person skilled in the art can easily deduce whether an antisense construct specifically interacts with/hybridizes to CHN-1/CHIP or CHN-1/CHIP orthologs encoding sequences. These tests comprise, but are not limited to hybridization assays, RNase protection assays, Northern Blots, North-western blots, nuclear magnetic resonance and fluorescence binding assays, dot blots, micro- and macroarrays and quantitative PCR. In addition, such a screening may not be restricted to CHN-1/CHIP or CHN-1/CHIP orthologs mRNA molecules, but may also include CHN-1/CHIP or CHN-1/CHIP orthologs mRNA/protein (RNP) complexes (Hermann, 2000; DeJong et al., 2002). Furthermore, functional tests as provided in the appended examples are envisaged for testing whether a particular antisense construct is capable of specifically interacting with/hybridizing to the CHN-1/CHIP or CHN-1/CHIP orthologs encoding nucleic acid molecules. These functional assays comprise ubiquitylation assays provided in the examples. These functional tests may also include Western blots, immunohistochemistry, immunoprecipitation assay, and bioassays based on, inter alia, GFP reporter gene fusions.

The term “antisense-molecule” as used herein comprises in particular antisense oligonucleotides. Said antisense oligonucleotides may also comprise modified nucleotides as well as modified internucleoside-linkage, as, inter alia, described in U.S. Pat. No. 6,159,697. Most preferably, the antisense oligonucleotides of the present invention comprise at least 8, more preferably at least 10, more preferably at least 12, more preferably at least 14, more preferably at least 16 nucleotides. The deduction as well as the preparation of antisense molecules is very well known in the art. The deduction of antisense molecules is, inter alia, described in Smith, 2000. Usual methods are “gene walking”, RNase H mapping, RNase L mapping (Leaman and Cramer, 1999), combinatorial oligonucleotide arrays on solid support, determination of secondary structure analysis by computational methods (Walton, 2000), aptamer oligonucleotides targeted to structured nucleic acids (aptastruc), thetered oligonucleotide probes, foldback triplex-forming oligonucleotides (FTFOs) (Kandimalla, 1994) and selection of sequences with minimized non-specific binding (Han, 1994).

Preferably, the antisense molecules of the present invention are stabilized against degradation. Such stabilization methods are known in the art and, inter alia, described in U.S. Pat. No. 6,159,697. Further methods described to protect oligonucleotides from degradation include oligonucleotides bridged by linkers (Vorobjev, 2001), minimally modified molecules according to cell nuclease activity (Samani, 2001), 2′O-DMAOE oligonucleotides (Prakash, 2001), 3′5′-Dipeptidyl oligonucleotides (Schwope, 1999), 3′methylene thymidine and 5-methyluridine/cytidine h-phosphonates and phosphonamidites (An, 2001), as well as anionic liposome (De Oliveira, 2000) or ionizable aminolipid (Semple, 2001) encapsulation.

In a preferred embodiment of the invention, the antisense molecule is a nucleic acid molecule which is the complementary strand of a reversed complementary strand of the coding region of CHN-1/CHIP or CHN-1/CHIP orthologs is selected from the group consisting of SEQ ID NO: 1, SEQ ID NO: 3, SEQ ID NO: 5, SEQ ID NO: 7 or SEQ ID NO: 9. Sequences as depicted in SEQ ID NOs: 1, 3, 5, 7, 9, 11 or 12 represent illustrative coding regions (mRNA) of C. elegans, human, zebrafish, mouse or rat, whereby SEQ ID NOs: 1 and 11 represent human sequences and SEQ ID NOs: 9 and 12 represent rat sequences. SEQ ID NOs: 2, 4, 6, 8 or 10 represent translated CHN-1/CHIP or CHN-1/CHIP orthologs of C. elegans, human, zebrafish, mouse or rat, respectively. Accordingly, in context of this invention and as stressed herein above, the CHN-1/CHIP or CHN-1/CHIP orthologs inhibitors to be employed in the uses described herein, preferably, interact with promoter and/or coding regions of nucleic acid molecules which code for or lead to the expression of CHN-1/CHIP or CHN-1/CHIP orthologs molecules as shown in SEQ ID NOs: 2, 4, 6, 8 or 10. It is also envisaged that, e.g., antisense constructs designed and used in accordance with this invention inhibit the expression of functional homologues, variants (for example allelic variants) or isoforms of CHN-1/CHIP or CHN-1/CHIP orthologs-molecules as shown in SEQ ID NOs: 2, 4, 6, 8 or 10.

In context of this invention, the term “coding region of CHN-1/CHIP or CHN-1/CHIP orthologs” comprises not only the translated region of CHN-1/CHIP or CHN-1/CHIP orthologs, but also comprises untranslated regions. Accordingly, the anti-CHN-1/CHIP or CHN-1/CHIP orthologs antisense molecule to be used and employed in accordance with this invention may be antisense molecules which bind to/interact with mRNA sequences comprising untranslated region.

The invention also relates to a method for preventing, ameliorating and/or treating a muscular disease comprising administering a specific inhibitor of CHN-1/CHIP or CHN-1/CHIP orthologs expression or function as defined herein above to a subject in need thereof. Preferably, said subject is a mammal, most preferably said mammal is a human.

A “patient” or “subject” for the purposes of the present invention includes both humans and other animals, particularly mammals, and other organisms. Thus, the methods are applicable to both human therapy and veterinary applications. In the preferred embodiment the patient is a mammal, and in the most preferred embodiment the patient is human.

Most preferably, the pharmaceutical composition to be prepared in accordance with this invention and comprising an anti-CHN-1/CHIP or CHN-1/CHIP orthologs expression and/or function inhibitor(s) is to be administered by one or several of the following modes: Administration can be oral, intravenous, intraarterial, intratracheal, intranasal, subcutaneous, intramuscular, intracranial (i.e. intraventricular) or intraspinal (intrathecal), epidermal or transdermal, pulmonary (e.g. inhalation or insufflation of aerosol or powder), by delivery to the oral or rectal mucosa as well as ophthalmic delivery. Preferred in context of this invention is a intramuscular administration.

Dosage and administration regimes for the CHN-1/CHIP or CHN-1/CHIP orthologs-inhibitors may be established by the physician. For example, for antisense compounds, like antisense-nucleotides specific dosage regimes have been established. Such regimes comprise a dosage of 1 mg/kg up to 200 mg/m² and are, inter alia, described in Schreibner (2001), Gastroenterology 120, 1399-1345; Andrews (2001), J. Clin. Oncol. 19, 2189-2200; Blay (2000), Curr. Op. Mol. Ther. 2, 468-472; Cunnigham (2000), Clin. Cancer Res. 6, 1626-1631; Waters (2000), J. Clin. Oncol. 18, 1809-1811 or Yacyshyn (1998), Gastroenterology 114, 1133-1142. It is, for example, envisaged that the CHN-1/CHIP or CHN-1/CHIP orthologs inhibitors described herein, e.g. antisense compounds, siRNAs or RNAi and the like, be administered in single doses of 0.1 to 25 mg/kg/die (for example i.v. over 2 to 8 hours), as single or multiple doses every other day or by continuous infusion(s) of 0.5 to 10 mg/kg/die over 14 to 21 days with 7 day rest. It is of particular note that in certain clinical or medical indications it might be desirable to administer the CHN-1/CHIP or CHN-1/CHIP orthologs-inhibitors as disclosed herein in a single dose. Further dosage regimes are envisaged and may easily be established by a physician.

In a most preferred embodiment the pharmaceutical composition, use or method of the invention, the CHN-1 ortholog or the CHIP ortholog is selected from the group consisting of a CHN-1- or CHIP-molecule encoded by a nucleotide sequence as shown in SEQ ID NO: 1 [C. elegans]; SEQ ID NO: 3 [human]; SEQ ID NO: 5 [zebrafish]; SEQ ID NO: 7 [mouse]; SEQ ID NO: 9 [rat], SEQ ID NO: 11 [human] or SEQ ID NO: 12 [rat].

Accordingly, CHN-1/CHIP molecules to be employed in context of this invention comprise, but are not limited to the molecules encoded by nucleic acid sequences as disclosed herein. Also envisaged are CHIP/CHN-1 orthologs which are at least 60%, 70%, 80%, 90%, 95%, 96%, 97%, 98% or 99%, most preferably at least 85% identical to the amino acid sequences as shown in SEQ ID NOs: 2, 4, 6, 8 or 10. It is of note that SEQ ID NOs: 1 and 11 code for human CHIP (see SEQ ID NO: 4) whereas SEQ ID NOs: 9 and 12 code for rat CHIP (see SEQ ID NO: 10).

Furthermore envisaged are CHIP/CHN-1 orthologs which are encoded by a nucleic acid sequence which is at least 60%, 70%, 80%, 90%, 95%, 96%, 97%, 98% or 99%, most preferably at least 85% identical to a polynucleotide as shown in SEQ ID NOs: 1, 3, 5, 7, 9, 11 or 12.

In addition, the term CHN-1/CHIP otholog comprises molecules which are at least 60%, more preferably at least 80% and most preferably at least 90% homologous to the polypeptide as shown in any of SEQ ID NOs: 2, 4, 6, 8 or 10.

The term “CHN-1/CHIP ortholog” also comprises functional fragments of the CHN-1/CHIP orthologs disclosed herein. A functional fragment is a fragment which is capable of ubiquitylation as described herein and the experimental part as well as the interaction with UFD2, the interaction with other E3 enzymes, the interaction with E2 enzymes or the interaction with UNC-45 or fragments of UNC-45.

In accordance with the present invention, the term “nucleic acid sequence” means the sequence of bases comprising purine- and pyrimidine bases which are comprised by nucleic acid molecules, whereby said bases represent the primary structure of a nucleic acid molecule. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

When used herein, the term “polypeptide” means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The terms polypeptide and protein are often used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature.

In order to determine whether a nucleic acid sequence has a certain degree of identity to the nucleic acid sequence encoding CHIP/CHN-1 orthologs, the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

The present invention also relates to nucleic acid molecules which hybridize to one of the above described nucleic acid molecules and which has CHIP/CHN-1 activity.

The term “hybridizes” as used in accordance with the present invention may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences having a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementartity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, as mentioned above, more preferably at least 60%, even more preferably at least 70%, particularly preferred at least 80%, more particularly preferred at least 90%, even more particularly preferred at least 95%, 97% or 98% and most preferably at least 99% identity with a nucleic acid sequence as described above encoding CHN-1/CHIP orthologs. Moreover, the term “hybridizing sequences” preferably refers to sequences encoding CHIP/CHN-1-orthologs of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with an amino acid sequence of CHN-1/CHIP-orthologs as described herein above.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands.

Moreover, the present invention uses and relates to nucleic acid molecules encoding CHIP/CHN-1-orthologs the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid.

The nucleic acid molecule encoding CHIP/CHN-1-orthologs may be any type of nucleic acid, e.g. DNA, RNA or PNA (peptide nucleic acid). A peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by Nielsen et al., Science 254:1497 (1991); and Egholm et al., Nature 365:666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the strong binding. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis. The DNA may, for example, be cDNA. In a preferred embodiment it is a genomic DNA. The RNA may be, e.g., mRNA. The nucleic acid molecule may be natural, synthetic or semisynthetic or it may be a derivative, such as peptide nucleic acid (Nielsen, Science 254 (1991), 1497-1500) or phosphorothioates. Furthermore, the nucleic acid molecule may be a recombinantly produced chimeric nucleic acid molecule comprising any of the aforementioned nucleic acid molecules either alone or in combination.

As pointed out above and as illustrated in the in vivo experiments provided herein the CHIP/CHN-1 inhibitors, antagonists or negative regulators are particularly useful in the treatment, amelioration or prevention of a muscular disease or a myopathy. Said myopathy or muscular disease is preferably a muscular dystrophy. Said muscular dystrophy may be selected from the group consisting of Duchenne muscular dystrophy (DMD), limb-girdle dystrophy, facio-scapulo-humeral dystrophy, dystrophia myotonica, muscular dystrophy, Becker type. Most preferred is, however, the treatment of DMD.

In a further embodiment of the invention a method for screening inhibitors, antagonists or negative regulators of CHN-1/CHIP activity, function or expression is provided, when said method comprises the steps of
(a) contacting a cell or a non-human organism expressing CHN-1/CHIP or capable of expressing CHN-1/CHIP with a compound to be tested;
(b) determining the status of ubiquitylation or multi-ubiquitylation in said cell or a cell of said non-human organism in the presence of said compound to be tested when compared to a cell not contacted with said compound; and
(c) identifying the compound which inhibits CHN-1/CHIP activity, function or expression.

The person skilled in the art is readily position to deduce/determine the ubiquitylation/multi-ubiquitylation status in a (cultured) cell or the cell of an organism. Corresponding examples are given in the experimental part of the invention.

The person skilled in the art can easily employ the compounds described herein and the methods of this invention in order to elucidate the inhibitory effects and/or characteristics of a test compound to be identified and/or characterized in accordance with any of the methods described herein and can deduce which is an inhibitor of CHN-1/CHIP ortholog function, activity or expression.

The term “test compound” or “compound to be tested” refers to a molecule or substance or compound or composition or agent or any combination thereof to be tested by one or more screening method(s) of the invention as a putative inhibitor of CHN-1/CHIP ortholog function, activity or expression. A test compound can be any chemical, such as an inorganic chemical, an organic chemical, a protein, a peptide, a carbohydrate, a lipid, or a combination thereof or any of the compounds, compositions or agents described herein. It is to be understood that the term “test compound” when used in the context of the present invention is interchangeable with the terms “test molecule”, “test substance”, “potential candidate”, “candidate” or the terms mentioned hereinabove.

Accordingly, small peptides or peptide-like molecules as described hereinabove are envisaged to be used in the screening methods for inhibitor(s) of CHN-1/CHIP ortholog, function, activity or expression. Such small peptides or peptide-like molecules bind to and occupy the active site of a protein thereby making the catalytic site inaccessible to substrate such that normal biological activity is prevented. Moreover, any biological or chemical composition(s) or substance(s) may be envisaged as CHN-1/CHIP inhibitor. The inhibitory function of the inhibitor can be measured by methods known in the art and by methods described herein. Such methods comprise interaction assays, like immunoprecipitation assays, ELISAs, RIAs as well as specific assays, like the assays provided in the appended Examples. In the context of the present application it is envisaged that cells expressing CHN-1/CHIP ortholog as described herein are used in the screening assays. It is also envisaged that elements of the pathway of ubiqitylation/multi-ubiquitylation as shown in the appended examples may be used, e.g., enzymes. Said enzymes may be present in whole cell extracts of cells expressing a CHN-1/CHIP ortholog or said enzymes may be purified, partially purified or recombinantly expressed as described hereinbelow.

Also preferred potential candidate molecules or candidate mixtures of molecules to be used when contacting a cell expressing a CHN-1/CHIP ortholog or an element of the ubiqitylation/multiubiquitylation pathway defined and described herein may be, inter alia, substances, compounds or compositions which are of chemical or biological origin, which are naturally occurring and/or which are synthetically, recombinantly and/or chemically produced. Thus, candidate molecules may be proteins, protein-fragments, peptides, amino acids and/or derivatives thereof or other compounds, such as ions, which bind to and/or interact with elements of the ubiqitylation/multi-ubiquitylation pathway defined herein, like the enzymes defined herein and/or fragments thereof which are described hereinbelow in the experimental part in detail. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means.

In addition, the generation of chemical libraries is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds can theoretically be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop, Journal of Medicinal Chemistry, Vol. 37, No. 9, 1233-1250 (1994)). Other chemical libraries known to those in the art may also be used, including natural product libraries. Once generated, combinatorial libraries are screened for compounds that possess desirable biological properties. For example, compounds which may be useful as drugs or to develop drugs would likely have the ability to bind to the target protein identified, expressed and purified as described herein.

In the context of the present invention, libraries of compounds are screened to identify compounds that function as inhibitors of the CHN-1/CHIP ortholog. First, a library of small molecules is generated using methods of combinatorial library formation well known in the art. U.S. Pat. Nos. 5,463,564 and 5,574,656 are two such teachings. Then the library compounds are screened to identify those compounds that possess desired structural and functional properties. U.S. Pat. No. 5,684,711, discusses a method for screening libraries. To illustrate the screening process, the target cell or gene product and chemical compounds of the library are combined and permitted to interact with one another. A labeled substrate is added to the incubation. The label on the substrate is such that a detectable signal is emitted from metabolized substrate molecules. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the cell/enzyme can be analyzed and features common to the various compounds identified can be isolated and combined into future iterations of libraries. Once a library of compounds is screened, subsequent libraries are generated using those chemical building blocks that possess the features shown in the first round of screen to have activity against the target cell/enzyme. Using this method, subsequent iterations of candidate compounds will possess more and more of those structural and functional features required to inhibit the function of the target cell/enzyme, until a group of (enzyme)inhibitors with high specificity for the enzyme can be found. These compounds can then be further tested for their safety and efficacy as antibiotics for use in animals, such as mammals. It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in PCT publications No. WO 99/35494, WO 98/19162, WO 99/54728.

Preferably, candidate agents to be tested in the encompass numerous chemical classes, though typically they are organic molecules, preferably small organic compounds having a molecular weight of more than 50 and less than about 2,500 Daltons, preferably less than about 750, more preferably less than about 350 daltons. Candidate agents may also comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Exemplary classes of candidate agents may include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

As mentioned above, candidate agents are also found among biomolecules including peptides, amino acids, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Other candidate compounds to be used as a starting point for the screening of inhibitors of CHN-1/CHIP orthologs are aptamers, aptazymes, RNAi, shRNA, RNAzymes, ribozymes, antisense DNA, antisense oligonucleotides, antisense RNA, antibodies, affybodies, trinectins, anticalins, or the like compounds which are described herein. These candidate compounds are, accordingly, also compounds to be employed in the pharmaceutical composition, uses and medical methods provided herein. Therefore, the person skilled in the art is readily in a position to have candidate compounds at his disposal which can be used in the screening methods for inhibitors of CHN-1/CHIP orthologs as a basis to, inter alia, improve or further develop the capability of such compounds to inhibit CHN-1/CHIP orthologs activities, function or expression. Accordingly, the person skilled in the art can readily modify such compounds by methods known in the art to improve their capability of acting as an inhibitor in the sense of the present invention. The capability of one or more of the aforementioned compounds to inhibit CHN-1/CHIP orthologs activities, function or expression is tested as described hereinabove.

In one embodiment of the present invention, the enzymes involved in ubiquitylation/multiubiquitylation as described herein are isolated and expressed. These recombinant proteins may then be used as targets or substrates in assays to screen libraries of compounds for potential drug candidates.

As mentioned above, also cell-based screening assays are within the scope of the present invention. Current cell-based assays used to identify or to characterize compounds for drug discovery and development frequently depend on detecting the ability of a test compound to modulate the activity of a target molecule located within a cell or located on the surface of a cell. Most often such target molecules are proteins such as enzymes and the like. A number of highly sensitive cell-based assay methods are available to those of skill in the art to detect binding and interaction of test compounds with specified-target molecules.

Current methods employed in the arts of medicinal and combinatorial chemistry are able to make use of structure-activity relationship information derived from testing compounds in various biological assays including direct binding assays and cell based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus, more compounds can now be characterized providing more useful information than would be obtained otherwise.

The non-human organism to be used in said screening method is preferably selected from the group consisting of C. elegans, yeast, drosophila, zebrafish, mouse, rat, guinea pig, dog, cat.

In a most preferred embodiment, said non-human organism to be employed is a transgenic organism. The C. elegans to be used may be, inter alia, a mutant in the dys-1 gene, or a strain with mutants in the dys-1 and hlh-1 genes, or a mutant in K08B12.5 (dmp-1), preferably a deletion of its coding region, or a point mutation reducing or altering its activity or expression, or a mutation in unc-60 (cofilin).

Also an embodiment of the invention is a method for the preparation of a pharmaceutical composition comprising the steps of
(a) identifying a compound capable of inhibiting or negatively-regulating CHN-1/CHIP activity by the method disclosed above; and
(b) formulating said compound with a pharmaceutically acceptable carrier.

The invention also relates to non-human transgenic animals which comprises a double mutation, wherein the first of said mutations comprises a modification in a gene which leads to a phenotype of a muscular disease. Wherein said first mutation is selected from the group consisting of a mutation in the Dystrophin-, Utrophin-, α-, β-, γ-, δ-Sarcoglycan-, Laminin-α2-, Dysferlin-, Integrin α5-, Integrin α7-, α-Dystrobrevin-, α-Dystroglycan-, Calpain 3-, Lamin A-, LARGE-, Caveolin 3-, CHIP-gene and wherein the second of said mutations comprises a mutation in a CHN-1/CHIP ortholog. Transgenic, non-human animals comprising modifications in genes which led to muscular diseases are known in the art and comprise but are not limited to:

| | | |
|---|---|---|
| Name | effected gene | reference |

Dystrophinopathies

| | | |
|---|---|---|
| mdx | Dystrophin | Sicinski (1989) Science 244: 1578-1580 |
| utrn (−/−) | Utrophin | Deconinck (1997) Cell 90: 717-727 |
| (mdx/utrn⁻) | dystrophin/utrophin- | Grady (1997) Cell 90: 729-738 |
| | double-Knockout | |
| mdx2-5^{CV} | Dystrophin | Cox (1993) Nature Genet. 4: 87-93 |

Sarcoglycanopathies

| | | |
|---|---|---|
| Sgca | α-sarcoglycan | Duclos (1998) J Cell Biol. 142: 1461-1471 |
| Sgcg | γ-sarcoglycan | Hack (1998) J Cell Biol. 142: 1279-1287 |
| Sgcb | β-sarcoglycan | Araishi (1999) Hum. Mol. Genet. 8: 1589-1598 |
| Sgcd | δ-sarcoglycan | Coral-Vazquez (1999) Cell 98: 465-474 |

Congenital muscular dystrophies

| | | |
|---|---|---|
| dy | laminin-α2 | Michelson (1955) Proc. Natl. Acad. Sci. USA 91: 1079-1084 |
| dy^{2J} | laminin-α2 | Xu (1994) Nature Genet. 8: 297-302 |
| dy^{3K} | laminin-α2 | Miyagoe (1997) FEBS Lett. 415: 33-39 |
| dy^{W} | laminin-α2 | Kuang (1998) J. Clin. Invest. 102: 844-852 |
| dy^{PAS} | laminin-α2 | Dubowitz (1999) Neuromusc. Disord. 9: 446-454 |

Dysferlinopathies

| | | |
|---|---|---|
| SJL | dysferlin | Bittner (1999) Nature Genet. 23: 141-142 |

other muscular dystrophies

| | | |
|---|---|---|
| α5 | integrin α5 | Taverna (1998) J. Cell Biol. 143: 849-859 |
| α7 | integrin α7 | Mayer (1997) Nature Genet. 17: 318-323 |
| adbn | α-dystrobrevin | Grady (1999) Nature Cell Biol. 1: 215-220 |
| Dag1 | α-dystroglycan | Williamson (1997) Hum. Mol. Genet. 6: 831-841 |
| capn3 | calpain 3 | Richard (2000) J. Cell Biol. 151: 1583-1590 |
| lmna (−/−) | lamin A | Sullivan (1999) J. Cell Biol. 147: 913-919 |
| LARGE | LARGE | Cane (1976) J. Hered. 67: 135-138 |
| (myd) | | |
| CAV3(−/−) | caveolin 3 | Galbiatii (2001) J. Biol. Chem. 276: 21425-21433 |
| CHIP (−/−) | chip | Dai (2003) EMBO J. 22: 5446-5458 |

Preferably, said non-human transgenic animal is a mouse, rat, zebrafish as well as drosophila. Said mutation is, preferably a CHN-1/CHIP ortholog “knock-out” mutation or a “conditioned knock-out” mutation, for example based on the cre/lox system. Corresponding knock-out animals (mice) are known in the art; see, e.g. Dai et al. (2003).

However, in particular in screening assays provided herein further or other mutations in the CHN-1/CHIP ortholog may be comprised in said double-transgenic non-human animal. Said mutation may, accordingly, also be an CHN-1/CHIP ortholog-overexpressing mutation or a mutation which leads to a more active CHN-1/CHIP ortholog. Also mutations in the CHN-1/CHIP ortholog which lead to a non-functional or less functional molecule are envisaged to be comprised in the double transgenic non-human animal provided here. Also envisaged is the use of single transgenic non-human animals in screening methods provided herein. Preferably, said single transgenic non-human animals are animals which overexpress the CHIP-ortholog or which lead to a more active CHN-1/CHIP ortholog function.

The non-human transgenic animals may be employed in screening methods disclosed herein. Particularly preferred are these animals (single or double transgenics described herein) in tests for inhibitors/antagonists/negative regulators of CHN-1/CHIP, CHN-1/CHIP-orthologs. Tests with these animals may comprise, but are not limited to: a) Force measurements; b) Histology; c) Immunhistochemistry; d) CK measurements or e) Membrane stability. These tests are, where applicable not limited to test with living animals but who comprise tests with and on organs, tissues or cells derived from said animals.

The technique to measure the force of animals comprises the measurement of e.g. grip strength. Furthermore the specific twitch force, the specific tetanic force, the resistance to eccentric contractions or the isometric tetanic force may be measured on isolated muscles. Said isolated muscles comprise for example EDL, quadriceps, tibialis anterior, musculus soleus and diaphragm.

Studies of the histology of tested animals can be performed, e.g. on muscle cross sections or Western blots. Dystrophic muscles show certain deviations from healthy muscles, which is characterized e.g. by polygonal shape and large equal cells with peripherally located nuclei. In order to determine the muscle fibre shape, H+E stainings on cross sections can be performed. Further histological analysis comprise Van Gieson staining for visualizing connective and fat tissue (fibrosis), nuclear bisbenzimide staining to evaluate central nucleation in dystrophic muscle or NADH-tetrazolium reductase staining reveals metabolic fiber type (oxidative versus, glycolytic fiber type).

Immunhistochemistry may be performed e.g. on muscle cross-sections to inspect the intactness of muscle membranes and can be assessed by checking expression and correct sarcolemmal membrane localization of molecules of the dystroglycan-sarcoglycan-complex which is absent/decreased in dystrophic muscle. Such immunhistochemistry comprises, inter alia, antibody stainings which reveal dystrophin and sarcoglycan complex membrane localization as well as the detection of dystrophic muscle which is permanently regenerated (by muscle stem cells, the so called satellite cells). Regeneration may be evaluated by immunostaining with NCAM-antibodies. Furthermore inflammatory cells invade and remove dystrophic tissue and can be detected e.g. by mac1-antibodies.

Also CK-measurement may be useful in context of the present invention and in particular in screening methods of the invention, since dystrophic muscle fiber reveals high serum creatine kinase (CK) levels.

Furthermore, membrane stability assays may also be employed in the method of the invention employing transgenic non-human animals described herein (or parts, like organs, tissues or cells thereof). For example “evan's blue” stains (‘leaky’) dystrophic muscle blue.

The screening methods for the elucidation, determination, verification of an antagonist/inhibitor/negative regulator of a mammalian ortholog of C. elegans CHIP-1 or of human CHIP (and preferably of human CHIP) may be optimized for high-through-put-screenings. These HTP-screenings are in particular useful when in vitro systems or cell-based systems (like altered muscle-cells) are employed. For example, and non-limiting, tests for ubiquitylation/multi-ubiquitylation may be carried out in an HTPS-system. As discussed above, the present invention further discloses a method of identifying a putative agent that inhibits CHN-1 expression or function, wherein the method comprises the steps of (a) contacting a C. elegans or isolated C. elegans cell (preferably a muscle cell) with an agent of interest, wherein the C. elegans or isolated C. elegans cell comprises a wild-type CHN-1 allele; and (b) observing the resulting phenotype of the C. elegans or isolated C. elegans cell, wherein modification of ubiquitylation, activity of CHIP ubiquitin ligase activity, myosin assembly or locomotion is indicative that the agent of interest is a putative agent that inhibits CHN-1/CHIP expression or function. These assays may also be carried out with relevant mutants of C. elegans, for example muscle dystrophy mutants (for example mutant in the dys-1 gene) as described in the experimental part. As documented in the appended examples, an CHIP/CHN-1 inhibitor may be screened on corresponding muscle degeneration/muscle dystrophy mutants and an increased locomotion rate of the mutant C. elegans may be indicative for a functional CHN-1/CHIP inhibitor. A further read-out in these assays and other assays is an inhibition of the ubiquitin-proteasome pathway, as illustrated in the examples. Accordingly, a (potential) CHN-1/CHIP-inhibitor leads, inter alia and for example, to a reduced ubiquitylation of UNC-45.

In an alternate embodiment of the invention, a putative CHN-1/CHIP inhibitory agent is identified by measuring the levels of expression of the CHN-1 allele following contact with the agent of interest, wherein a reduced level of CHN-1 expression as compared to a suitable control (i.e., expression levels measured in a wild-type C. elegans nematode, or in a cell derived from a wild-type C. elegans nematode, in each case untreated with the agent of interest) indicates that the agent of interest is a putative agent that inhibits CHN-1 (and potentially also mammalian/human CHIP) expression.

It is also feasible that nematodes and in a particular also C. elegans wildtype or mutants be used in high-through put screenings. By automating high throughput screening and scoring in an array format, it is possible to gain a significant increase in throughput, a reduction in investigator time, and an increase in objectivity and accuracy. The array format may be managed by existing high throughput plate-based robotics. For example, high-throughput screenings using nematodes have been described in WO00/63427, relating to screening methods using nematode worms, particularly but not exclusively C. elegans, which are adapted to be performed in a high-throughput format. A further high-through put screening for nematodes applying an automated worm handling technology has been described in WO2004/033654. Also for the dys-deficient C. elegans HTPS-systems have been described, in order to test/assay drug effects, see for example Gaud (2004) Neuromuscular Disorders 14, 365-370. HTPS systems in DMD research have also been used and were described in Khurana (2003) Nature Reviews drug discovery 2: p 379-390 in particular in mouse systems.

The figures show:

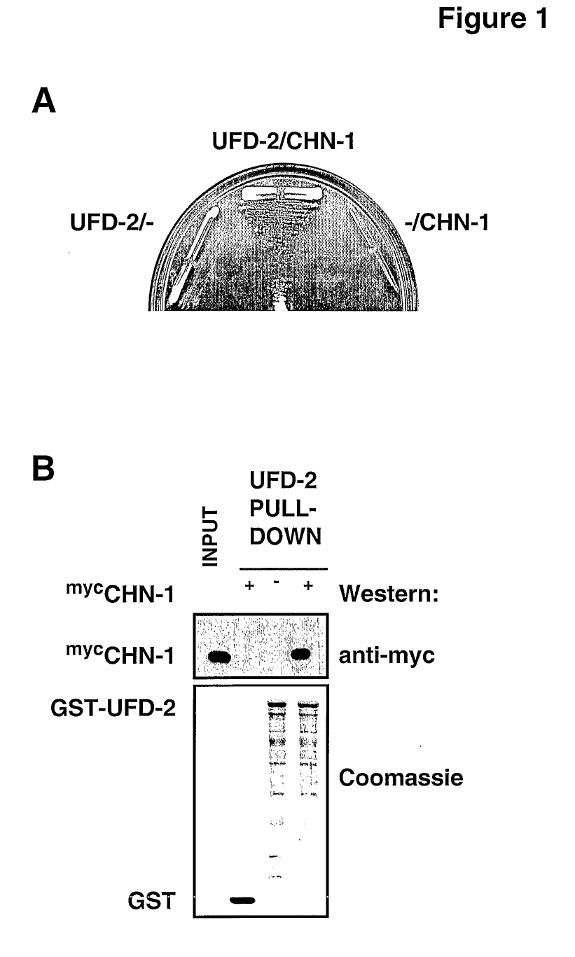

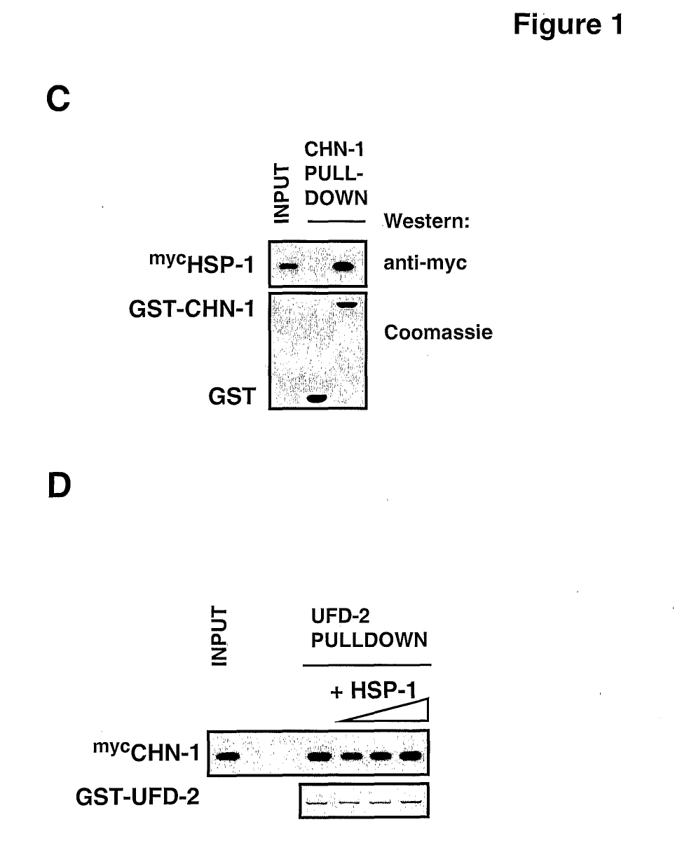

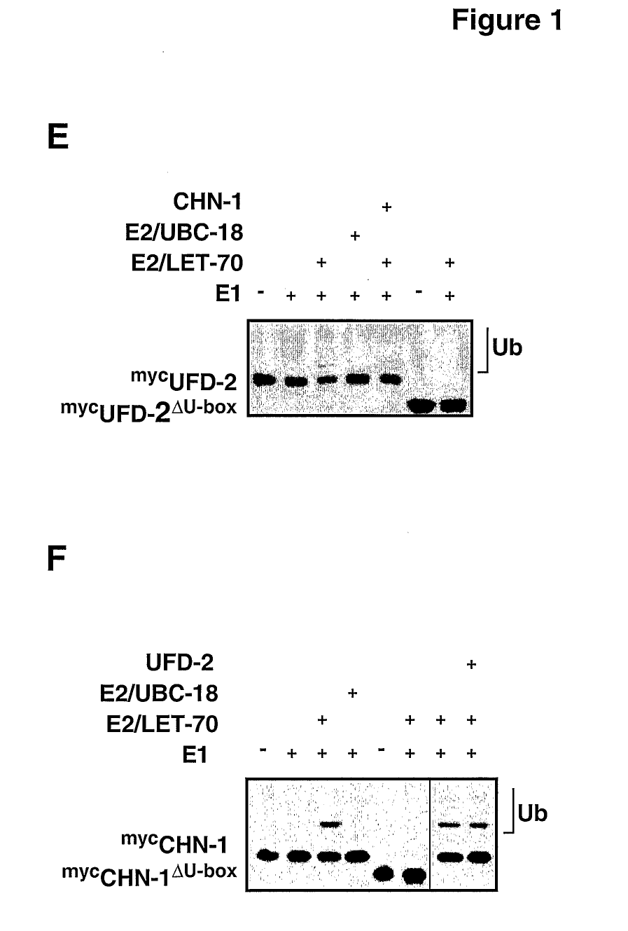

FIG. 1: The C. elegans CHIP Ortholog CHN-1 Binds to UFD-2

(A) Two-hybrid assay for the interaction of UFD-2 with CHN-1. Yeast cells expressing the indicated proteins were streaked out on medium plates lacking histidine to test for interaction-dependent activation of the HIS3 gene.

(B-D) Pulldown experiments with recombinant proteins followed by Western blotting using anti-myc antibodies and by Coomassie Blue staining.

(B) Interaction of CHN-1 and UFD-2 in vitro. Bacterially expressed myc-tagged CHN-1 (^{myc}CHN-1) was incubated with immobilized GST or immobilized GST-UFD-2.

(C) Interaction of the Hsp70 ortholog HSP-1 with CHN-1 in vitro. Bacterially expressed myc-tagged HSP-1 (^{myc}HSP-1) was incubated with immobilized GST or immobilized GST-CHN-1.

(D) HSP-1 does not compete for binding of CHN-1 with UFD-2. ^{myc}CHN-1 was allowed to bind to GST-UFD-2 in the presence of HSP-1 in increasing concentrations (0, 0.3, 3, and 30 μg respectively).

(E) Self-ubiquitylation of UFD-2 and CHN-1. Recombinant myc-tagged versions of UFD-2 or UFD-2 AU-box and CHN-1 or CHN-1^{ΔU-box} were incubated with the combination of enzymes indicated. The reaction was terminated by addition of SDS-PAGE sample buffer and analyzed by Western blotting. Efficient self-ubiquitylation of UFD-2 and CHN-1 requires E1, the E2 enzyme LET-70 and a functional U-box.

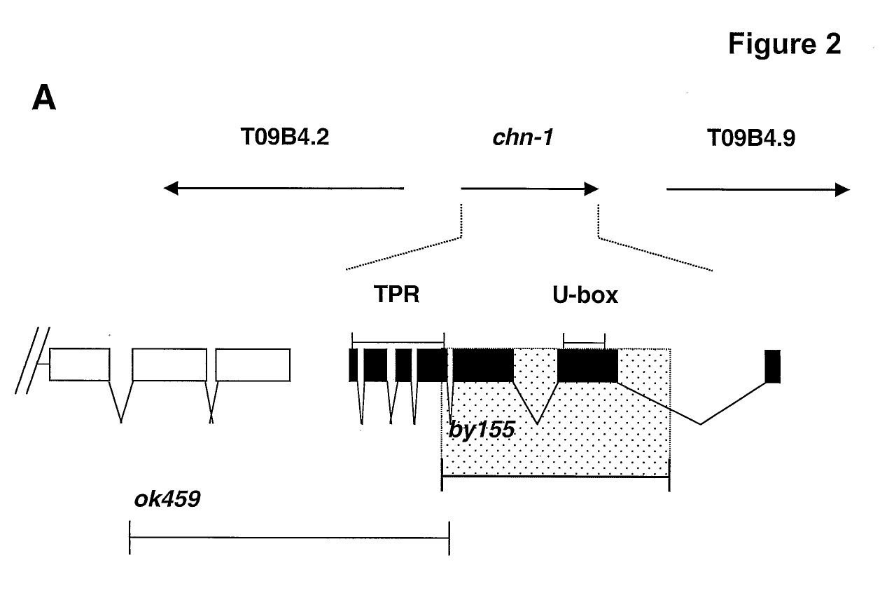

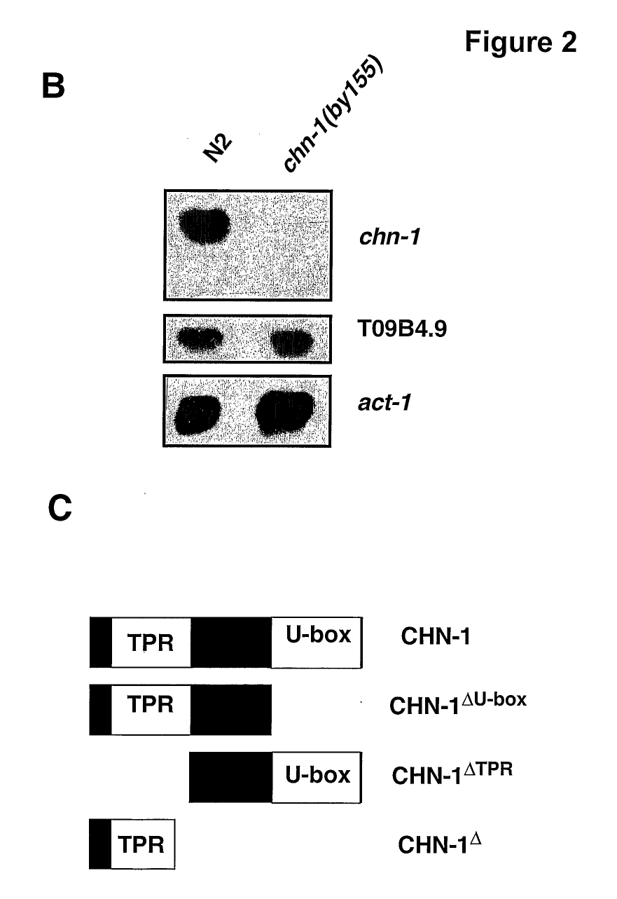

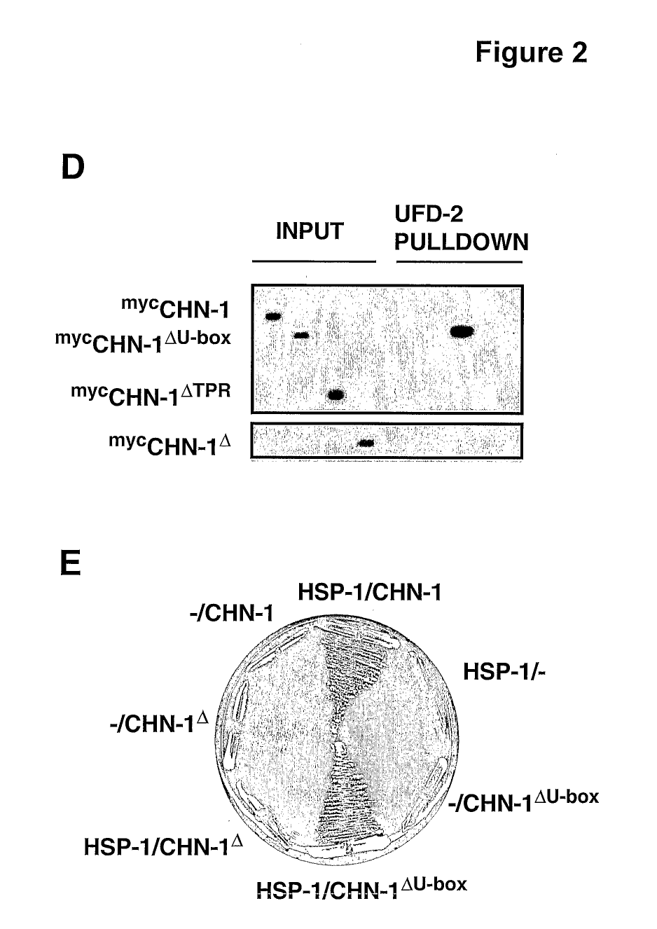

FIG. 2: Deletion of chn-1

(A) Isolation of the chn-1(by155) deletion allele. The chn-1 gene (T09B4.10) maps to chromosome 1. The genomic regions encoding the different domains are shown: TPR motifs (TPR) and U-box domain (U-box). The position and extent of the deletion alleles by155 (Δ989 bp) and ok459 (Δ1422 bp) are indicated.

(B) Northern blot analysis of the chn-1 and T09B4.9 transcripts from N2 wild-type and chn-1(by155). The blot was probed with a chn-1 specific cDNA fragment and with a specific DNA fragment against the neighbouring gene T09B4.9, which is part of the same operon as chn-1. An act-1 specific probe was used as loading control.

(C) Diagram of CHN-1 and deletion variants used in (D) and (E). Full-length CHN-1 possesses three TPR motifs and a U-box domain.

(D) The TPR motifs are required for binding of CHN-1 with UFD-2. As in (1B), the fragments of CHN-1, ^{myc}CHN-1^{ΔU-box}, ^{myc}CHN-1^{ΔTPR} and ^{myc}CHN-1^{Δ} (corresponding to the chn-1(by155) deletion) were tested for their ability to bind to GST-UFD-2.

(E) The CHN-1 fragment, CHN-1^{Δ}, corresponding to the chn-1(by155) deletion, does not interact with HSP-1. Two-hybrid interactions of CHN-1 and the truncated versions, CHN-1^{ΔU-box} and CHN-1^{Δ}, were tested for binding to HSP-1 on medium plates lacking histidine.

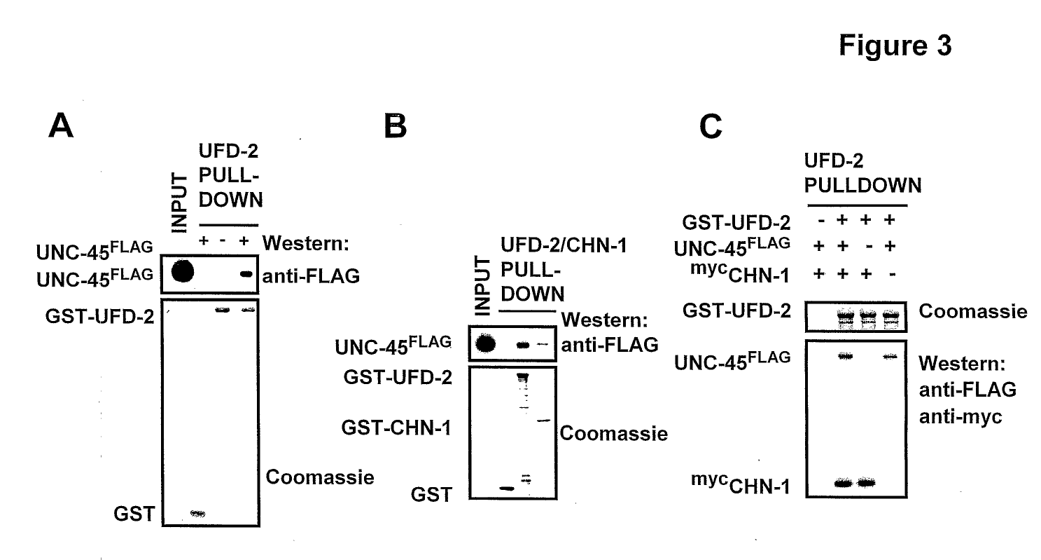

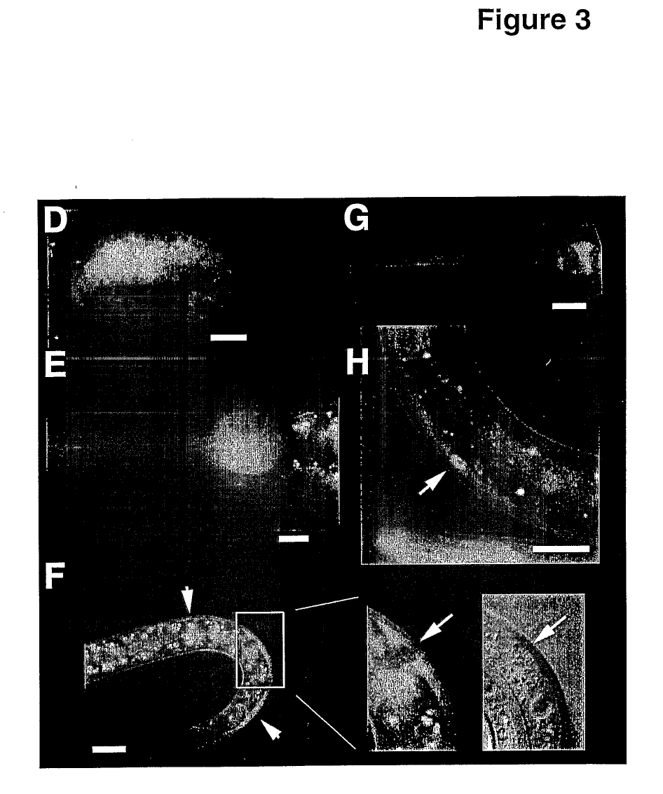

FIG. 3: Complex of CHN-1 UFD-2 and UNC-45

(A-C) Pulldown experiments with recombinant proteins followed by Western blot analysis and Coomassie Blue staining.

(A) Interaction of UFD-2 and UNC-45 in vitro. GST-UFD-2 was incubated with purified FLAG-tagged UNC-45 (UNC-45^{FLAG}) and pulled down with glutathione-Sepharose beads. GST alone with UNC-45^{FLAG} served as the negative control.

(B) In vitro binding of UNC-45 by UFD-2 or CHN-1. Equimolar amounts of GST-UFD-2 and GST-CHN-1 were incubated with the same amount of purified FLAG-tagged UNC-45 (UNC-45^{FLAG}) and pulled down with glutathione-Sepharose beads. GST alone with UNC-45^{FLAG} served as the negative control.

(C) Complex of CHN-1, UFD-2 and UNC-45. GST-UFD-2 was incubated with purified FLAG-tagged UNC-45 (UNC-45^{FLAG}) and myc-tagged CHN-1 (^{myc}CHN-1) together, ^{myc}CHN-1 or UNC-45^{FLAG} alone and pulled down with glutathione-Sepharose beads. GST alone with UNC-45^{FLAG} and ^{myc}CHN-1 served as the negative control.

(D-H) Transgenic expression of chn-1 and ufd-2 in muscle cells of N2 wild-type animals. (D) GFP::chn-1 is expressed in the cytoplasm of most cells of embryos at the twofold stage, (E) in pharynx muscles, (F) and in body wall muscle cells (arrows) at different larval stages. (G) ufd-2::GFP is expressed in pharynx muscles, and (H) in the cytoplasm and the nuclei of body wall muscle cells (arrow). Scale bars: 10 μm.

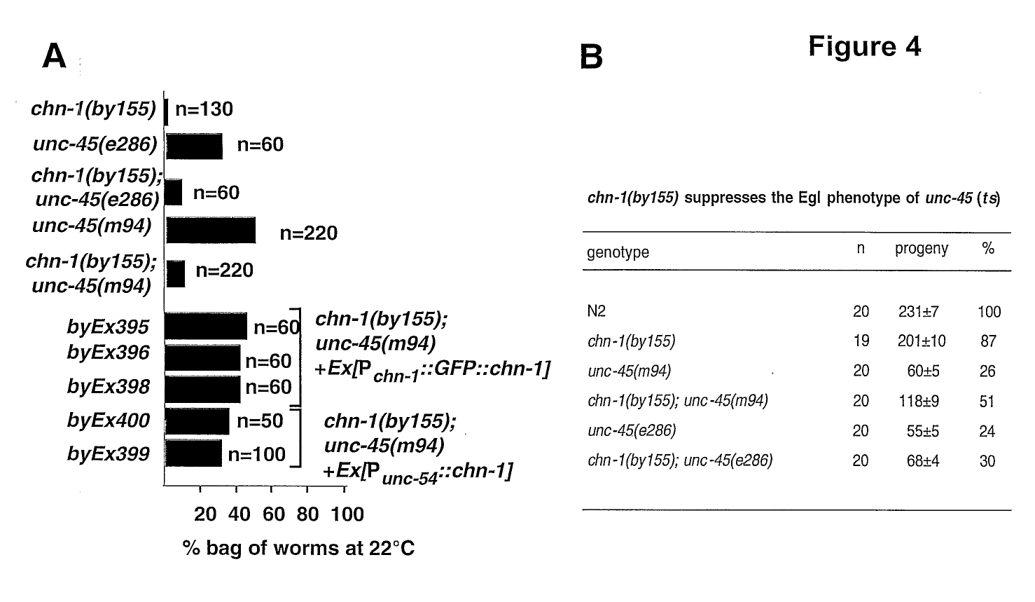

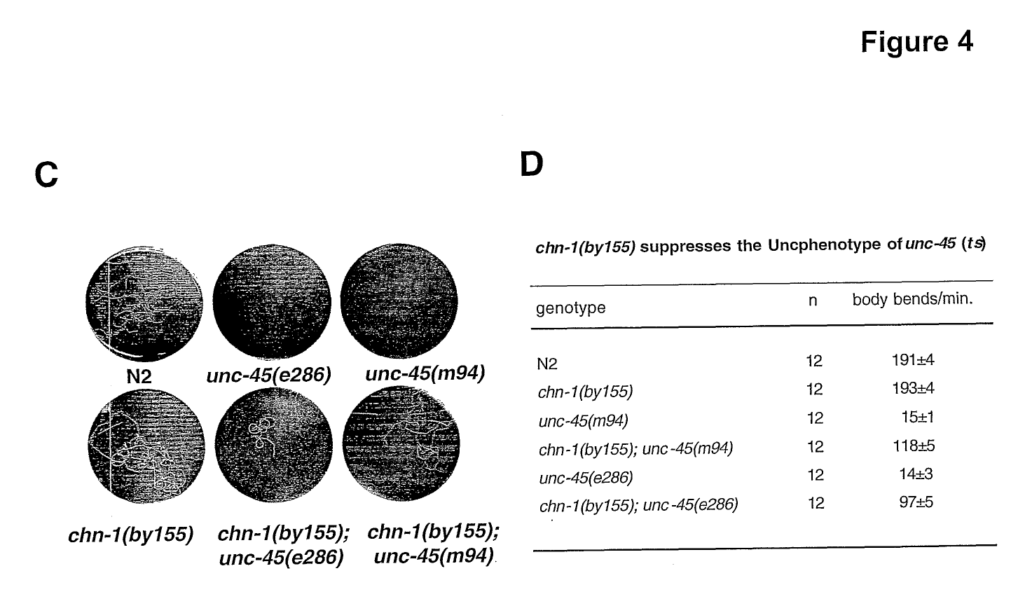

FIG. 4: Genetic Interaction of chn-1 with unc-45

(A) Suppression of the ‘bag of worms’ (Bag) Phenotype of unc-45 (ts) mutants by chn-1(by155). L3 larvae of the indicated strains were shifted from 15° C. to 22° C. 48 hr before counting the number of animals that died of internal hatching (‘bag of worms’ in %). This suppression was reverted by extrachromosomal expression of GFP::chn-1 under the chn-1 promoter (P_{chn-1}::GFP::chn-1) or by extrachromosomal expression of the chn-1 cDNA under the muscle specific unc-54 promoter (P_{unc-54}::chn-1). Three (P_{chn-1}::GFP::chn-1: byEx395, byEx396, byEx398) and two (P_{unc-54}::chn-1: byEx399, byEx400) independent lines were analyzed for rescue of chn-1(by155) dependent suppression of unc-45(m94).

(B) Suppression of the egg-laying defect (Egl) of unc-45 (ts) mutants by chn-1(by155). L3 larvae of chn-1(by155), unc-45(m94), unc-45(e286) single and double mutants were shifted from 15° C. to 22° C. and progenies were counted.

(C) Suppression of the Unc Phenotype of unc-45 (ts) mutants by chn-1(by155). The bacterial lawns on the plates show traces of temperature shifted worms (shifted as in 4A and 4B) after crawling for 1 h at 22° C. for chn-1(by155), unc-45(m94), unc-45(e286) single and double mutants. Ten young adults were assayed for each strain and all displayed similar motility.

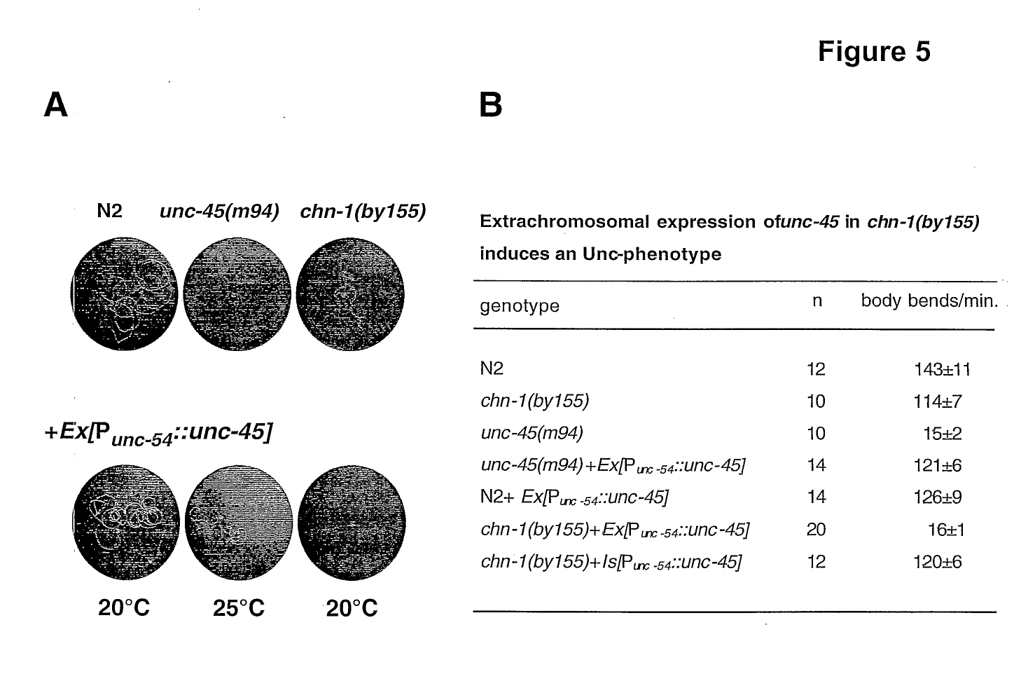

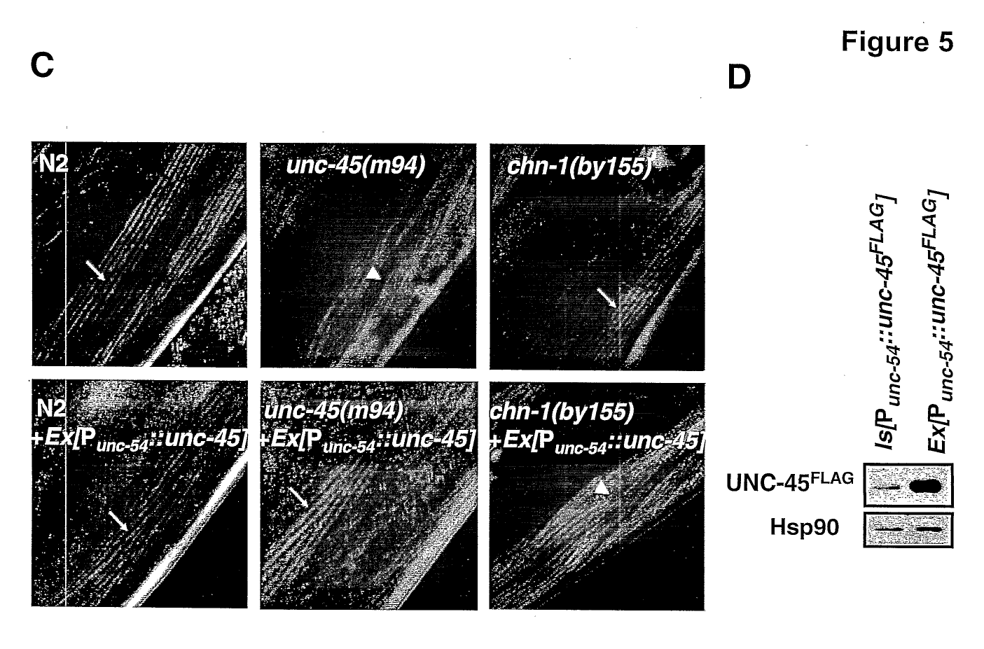

FIG. 5: Extrachromosomal Expression of unc-45 in chn-1(by155)

(A) Motility assay for N2 wild-type, unc-45(m94) and chn-1(by155). The bacterial lawns on the plates showing traces of worms after crawling for 1 h at 20° C. or 25° C. for N2, unc-45(m94) and chn-1(by155) with or without extrachromosomal expression of unc-45 under the muscle specific unc-54 promoter (P_{unc-54}::unc-45). Ten young adults were assayed for each strain and all displayed similar motility.

(B) Measurement of body bends for N2 wild-type, unc-45(m94) and chn-1(by155). The number of body bends per minute of young adult animals grown at 25° C. were counted for N2, unc-45(m94) and chn-1(by155) with or without extrachromosomal expression of unc-45 under the muscle specific unc-54 promoter (P_{unc-54}::unc-45).

(C) Polarized light microscopy of body wall muscles of N2, unc-45(m94) and chn-1(by155) strains expressing or not unc-45 under the muscle specific unc-54 promoter (P_{unc-54}::unc-45). All micrographs are of muscle cells located immediately posterior to the pharynx of young adult animals grown at 25° C. Note that the muscle cells of N2, chn-1(by155), N2+P_{unc-54}::unc-45, and unc-45(m94)+P_{unc-54}::unc-45 animals have well organized sarcomeres in which long A bands (bright bands, marked by arrows) alternate with long I bands (dark bands), whereas those of unc-45(m94) and chn-1(by155)+P_{unc-54}::unc-45 animals have disorganized sarcomeres in which alternating A and I bands are difficult to identify in equivalent regions (arrowheads).

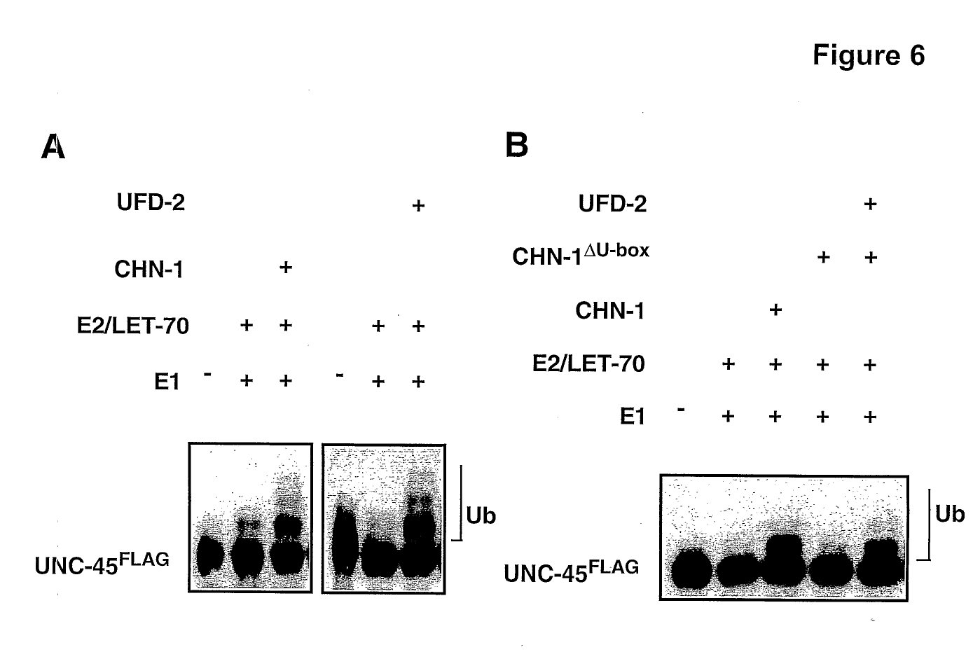

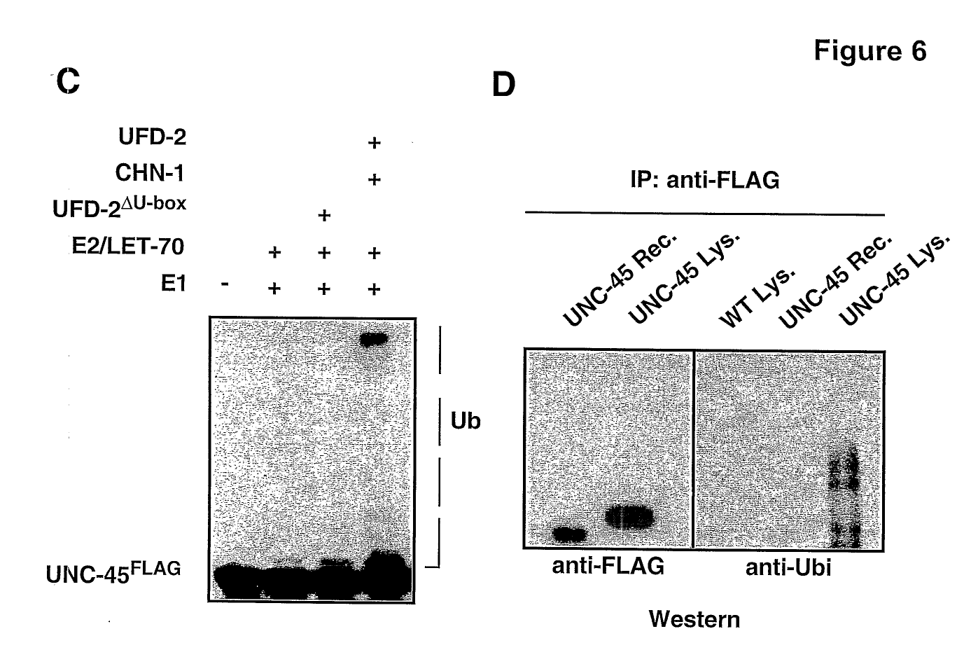

FIG. 6: UNC-45 is a Target for CHN-1 and UFD-2 Dependent Ubiguitylation

(A-C) In vitro ubiquitylation of UNC-45. Purified FLAG-tagged UNC-45 (UNC-45^{FLAG}) was incubated with the combination of enzymes indicated. The reaction was terminated by addition of SDS-PAGE sample buffer and analyzed by Western blotting. (A) UNC-45 ubiquitylation requires CHN-1 or UFD-2. (B) The ubiquitylation of UNC-45 depends on a functional U-box of CHN-1. (C) Both CHN-1 and UFD-2 together multiubiquitylate UNC-45.

(D) UNC-45 is ubiquitylated in vivo. Recombinantly expressed FLAG-tagged UNC-45 (UNC-45 Rec.) or UNC-45^{FLAG} expressed in N2 wild-type animals (UNC-45 Lys.) were immunoprecipitated with FLAG-specific antibodies and the precipitates were separated by SDS gel electrophoresis followed by Western blotting using anti-FLAG antibodies or anti-Ubiquitin (anti-Ubi) antibodies. Lysate of N2 wild-type which did not express FLAG-tagged UNC-45 (WT Lys.) served as the negative control and did not react with anti-Ubi antibodies.

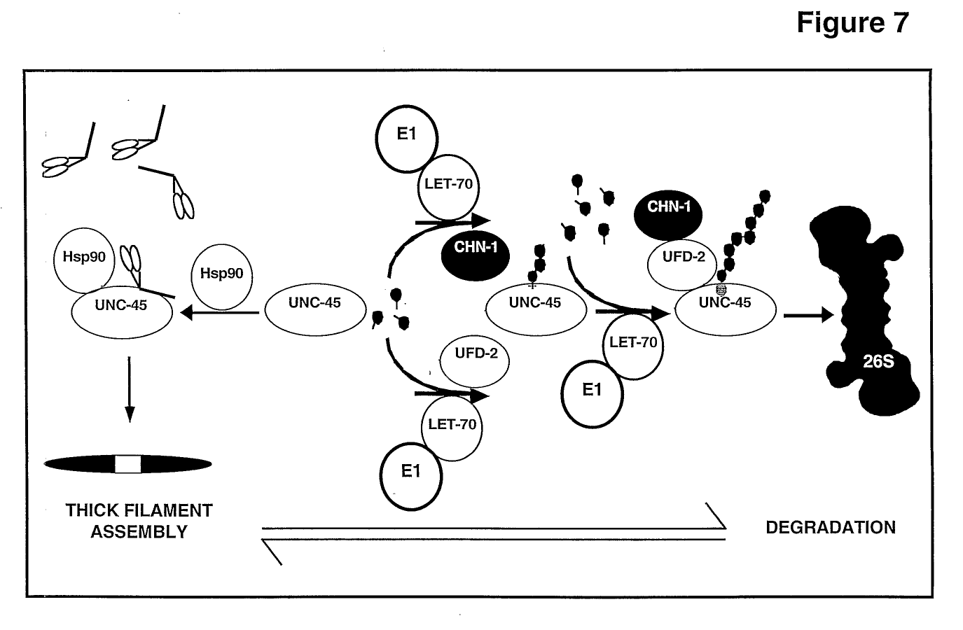

FIG. 7: Hypothetical Model for CHN-1/UFD-2 Dependent Regulation of the Myosin Chaperone UNC-45

UNC-45 is able to bind myosin and Hsp90 simultaneously and thereby functions both as a molecular chaperone and as an Hsp90 co-chaperone for myosin in muscle thick filament assembly. In collaboration with the ubiquitin-activating enzyme (E1) and the ubiquitin-conjugating enzyme (E2) LET-70, the E3/E4-complex formed by CHN-1 and UFD-2 multiubiquitylates UNC-45. Multiubiquitylated UNC-45 is probably degraded by the 26S proteasome. Thus, the protein level of the myosin chaperone UNC-45 seems to be tightly regulated which appears to be necessary for the correct assembly of myosin in body wall muscle cells.

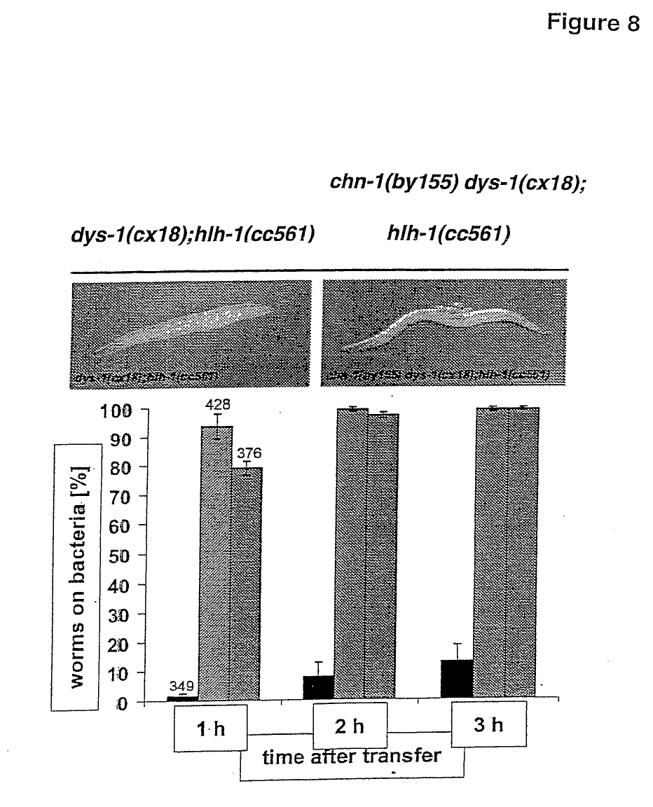

FIG. 8: chn-1 Deletion Increases Mobility of dys-1(cx18);hlh-1(cc561) Background C. elegans

chn-1 background rescue the muscle degeneration in dys-1(cx18);hlh-1(cc561) worms. The chn-1(by155) dys-1(cx18);hlh-1(cc561) adult animals show wild-type-like body shape and a lower egg number in the uterus when compared to dys-1(cx18);hlh-1(cc561) adults.

chn-1(ok459) and chn-1(by155) deletion increase the mobility of dys-1(cx18);hlh-1(cc561). Adult worms were placed on agar plates with fresh bacteria at the opposite side of the plate as odourus attraction and after a specified time period the percentage of animals on bacteria was calculated. The chn-1(ok459) dys-1(cx18);hlh-1(cc561) (grey dotted column) and chn-1(by155) dys-1(cx18);hlh-1(cc561) (grey column) triple mutants showed a highly increased locomotion when compared to dys-1(cx18);hlh-1(cc561) (black column). The horizontal axis shows the time in hours after the animals had been transferred to the assay plate. The results are based on at least four independent experiments. The total number of tested animals per strain is indicated on top of the first column.

The invention will now be described by reference to the following biological examples which are merely illustrative and are not construed as a limitation of the scope of the present invention.

Before the present invention is described in detail, it is to be understood that this invention is not limited to the particular methodology, protocols, cells, animals, vectors, reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

FIG. 9: Illustrative examples for siRNAs to be employed in context of this invention. A human; B mouse

### EXAMPLE 1

### Materials and Methods

### Strains

Worms were handled according to standard procedures and grown at 20° C. unless otherwise stated (Brenner, 1974). The mutations used in this study are listed by chromosomes as follows: LGI: chn-1(by155), chn-1(ok459), (?)ok59, unc-54(e1301); LGIII: unc-45(e286), unc-45(m94), unc-45(st604), daf-7(n696). The bristol strain N2 was used as the wild-type strain. The chn-1(ok459) mutant was kindly provided by the C. elegans Gene Knockout Consortium, Oklahoma.

### Plasmids

chn-1 and ufd-2 were amplified by PCR and cloned into pGBKT7 (pGBK-UFD-2), pGADT7 (pGAD-CHN-1) and pGEX4T1 (pGEX-CHN-1, pGEX-UFD-2). A myc-tag was introduced 5′ of chn-1, ufd-2 and hsp-1 cDNAs by PCR and cloned into pET21a (pET-^{myc}CHN-1, pET-^{myc}UFD-2, pET-^{myc}HSP-1). pET-^{myc}CHN-1 and pGAD-CHN-1 have been modified to create the deleted versions pET-^{myc}CHN-1^{ΔU-box}, pET-^{myc}CHN-1^{ΔTPR}, pET-^{myc}CHN-1^{Δ} and pGAD-^{myc}CHN-1^{ΔU-box}, pGAD-^{myc}CHN-1^{Δ}. The same was done with pET-^{myc}UFD-2 to obtain ^{myc}UFD-2^{ΔU-box}. hsp-1 was subcloned into pGBKT7 (pGBK-HSP-1). The cDNAs encoding the E2 enzymes LET-70 and UBC-18 were cloned into pGEX4T1 (pGEX-LET-70, pGEX-UBC-18). Details on each construct are available on request.

### Transgene Construction

The ufd-2::GFP transgene was constructed by subcloning 1.2 kb upstream regulatory sequence of the predicted start codon and the complete 3.2 kb genomic ufd-2 locus (predicted gene T05H10.5) into vector pPD95.75 (gift of A. Fire) in frame to a 3′ GFP cassette. The resulting construct was coinjected at a final concentration of 80 ng/μl with 20 ng/μl of the marker plasmid pRF4 (rol-6) (Mello, 1991). GFP::chn-1 was constructed by subcloning 0.6 kb predicted promoter sequence upstream and the complete 2.9 kb genomic chn-1 locus (predicted gene T09B4.10) downstream in frame to a GFP cassette into vector pPD117.01 (gift of A. Fire). The resulting construct was coinjected at a final concentration of 20 ng/μl with 20 ng/μl of the marker plasmid pRF4 (rol-6). At least three independent transgenic lines were analyzed to determine the expression pattern of ufd-2::GFP and GFP::chn-1. The same pPD117.01-P_{chn-1}::GFP::chn-1 construct was used for rescue experiments. Additionally, the chn-1 cDNA was subcloned into pPD88.27 to get pPD88.27-P_{unc-54}::chn-1. For overexpression studies the unc-45 cDNA containing a 3′FLAG-tag sequence was subcloned into pPD30.38 and 20 ng/μl of the resulting construct pPD30.38-P_{unc-54}::unc-45^{FLAG} were coinjected with 20 ng/μl of the marker plasmid pPD114.108 (mec-7::GFP).

### Yeast Two-Hybrid Studies

The full-length UFD-2 protein was fused to the GAL4 DNA binding domain using the vector pGBKT7 and transformed in the yeast host strain AH109 (James, 1996), obtained from CLONTECH (Palo Alto, Calif.). Protein interaction studies were carried out according to the manufacturer's instructions.

### Expression and Purification of Proteins

BL21(pRIL) E. coli cells were used to produce recombinant enzymes and lysed in buffer A [10 mM Tris pH 8; 10 mM DTT, Complete™ protease inhibitors (Boehringer)]. For purification of GST fusion proteins (GST-UFD-2, GST-CHN-1, GST-LET-70, GST-UBC-18) cleared lysates were allowed to bind to 500 μg of glutathione-Sepharose beads (Pharmacia). After extensive washing in buffer A with 0.1% Triton X-100 and 500 mM NaCl, GST-fusion proteins were eluted with 10 mM glutathione. UNC-45 was purified as described (Barral, 2002).

### Binding Assays and Immunoprecipitation

Binding of UNC-45^{FLAG}, ^{myc}CHN-1, ^{myc}CHN-1^{ΔU-box}, ^{myc}CHN-1^{ΔTPR}, ^{myc}CHN-1^{Δ}, ^{myc}UFD-2, ^{myc}UFD-2^{ΔU-box,} ^{myc}HSP-1 to GST-UFD-2 or GST-CHN-1 was done in buffer A [10 mM Tris pH 8, 10 mM DTT, Complete protease inhibitors (Boehringer)]+150 mM NaCl at 4° C. for 60 min. Glutathione-Sepharose beads were washed at least five times in buffer A with 0.1% Triton X-100 and 150 mM NaCl and bound proteins were eluted with SDS-PAGE sample buffer. Sonicated lysates from wild-type worms expressing the transgene P_{unc-54}::unc-45^{FLAG} were incubated with anti-FLAG M2-agarose beads (Sigma) in 50 mM Tris/HCl (pH 8.0), 100 mM NaCl, 10% glycerol, 1% Triton X-100 and 5 mM PMSF at 4° C. for 3 hours. Samples were precipitated with protein G-agarose beads (Amersham Pharmacia). Immunoprecipitates were washed at least five times in the same lysis buffer, were eluted with SDS-PAGE sample buffer and were separated by SDS gel electrophoresis followed by Western blotting using anti-FLAG M2 monoclonal antibodies (Sigma) or anti-Ubiquitin monoclonal antibodies (Chemicon).

### In Vitro Ubiquitylation Assay

Reactions were done as previously described (Koegl, 1999). Purified rabbit E1 (Affiniti), LET-70, UBC-18 and ^{myc}CHN-1, ^{myc}CHN-1^{ΔU-box}, ^{myc}CHN-1^{ΔTPR}, ^{myc}CHN1^{Δ}, ^{myc}UFD-2, ^{myc}UFD-2^{ΔU-box}, ^{myc}HSP-1 crude E. coli cell extracts were used for self-ubiquitylation reactions and for in vitro ubiquitylation of purified UNC-45.

### Northern Blot Analysis

RNA was isolated from mixed stage worm plates and prepared with an RNAeasy kit according to the manufacturer's instructions (Qiagen, Hilden). 5 μg of total RNA were loaded per lane and blots were performed following standard procedures (Lakowski, 2003). Probes were labelled with α³²P dCTP using the Megaprime labelling kit according to the manufacturer's instructions (Amersham). An act-1 specific probe was used as a loading control.

Isolation of the chn-1(lg155) Deletion Mutant

Nested PCR was used to screen for shorter alleles of chn-1 in an EMS mutagenized worm library that was composed of 3.5×10⁶ mutagenized genomes. The wild-type chn-1 allele amplified a 2920 bp fragment compared to a 1931 bp fragment from the chn-1(by155) deletion allele (Deletion breakpoints (T09B4 coordinates): 32756/33746 through 32757/33747). The chn-1(by155) mutant strain was backcrossed eight times to animals of wild-type background. For rescue experiments 20 ng/μl of the subclone pPD117.01-P_{chn-1}::GFP::chn-1 or pPD88.27-P_{unc-54}::chn-1 were coinjected into chn-1(by155); unc-45(m94) animals with 20 ng/μl of the marker plasmid pRF4 (rol-6) or pBY1153 (sel-12::GFP). Three (byEx395, byEx396, byEx398) and two (byEx399, byEx400) independent lines respectively were analyzed for rescue of chn-1(by155) dependent suppression of unc-45(m94).

### Motility Assay

Ten young adult worms of each strain were put in the center of ten plates that were completely seeded with a lawn of E. coli cells (OP50). The worms were allowed to crawl for 1 h, and the traces were photographed. In each experiment all ten worms of the same strain showed similar motility.

### Software and Microscopy

Quantitative evaluation of Northern blots was performed using ImageQuant 5.0 software (Molecular Dynamics). Photographs of GFP-expression were taken and polarized light microscopic examination of body wall muscle was carried out with an AxioPlan 2 Microscope (Zeiss) using the AxioVision 3.0 software.

### EXAMPLE 2

### CHN-1 and UFD-2 Directly Interact with Each Other and Both Collaborate with the E2 Enzyme LET-70

A C. elegans homolog of yeast UFD2 (Koegl, 1999) in the C. elegans database (open reading frame T05H10.5) was identified, encoding a U-box-containing protein with an overall identity of 26%, and it was named ufd-2. To investigate the role of UFD-2 in C. elegans, it was searched for proteins that interact with UFD-2 using the yeast two-hybrid system and CHN-1, a homolog of the human E4 enzyme CHIP (Cyr, 2002) (FIG. 1A) was identified. Furthermore, a Glutathione S-Transferase (GST) pull-down assay was carried out. It was found that bacterially expressed myc-tagged CHN-1 (^{myc}CHN-1) interacted with a GST-UFD-2 protein purified from E. coli, and vice versa (FIG. 1B and data not shown). Additional two-hybrid analysis (FIG. 2E) and in vitro protein binding assays showed that the C. elegans Hsp70 homolog HSP-1 (Heschl, 1990) is able to interact with CHN-1 (FIG. 1C) and does not compete for UFD-2 binding (FIG. 1D).

Self-ubiquitylation is characteristic for enzymes that belong to the ubiquitin system (Lorick, 1999). In order to study the enzymatic activities of UFD-2 and CHN-1, an assay in which self-ubiquitylation in the absence of a specific substrate was performed and revealed ubiquitin conjugation that occurred on the ubiquitin ligase itself. Bacterially expressed UFD-2 or CHN-1 were incubated in separate reactions containing ubiquitin, E1 and the purified C. elegans E2 enzymes LET-70 or UBC-18. Both E2 enzymes are homologs of yeast UBC4, which functions in the same degradation pathway as yeast UFD2 (Jones, 2002; Koegl, 1999). It has been shown that CHIP also functions specifically with the E2 enzymes of the UBC4/5 family (Jiang, 2001; Murata, 2001). Accordingly, E2 enzymes cooperate with UFD-2. Surprisingly, myc-tagged UFD-2 (^{myc}UFD-2) showed self-ubiquitylation only in conjunction with the E2 enzyme LET-70 and not with UBC-18 (FIG. 1E, top). CHN-1 self-ubiquitylation showed the same dependency on LET-70 (FIG. 1E, bottom). In comparison to full-length UFD-2 or CHN-1, the truncated proteins lacking the U-box had no self-ubiquitylation activity (FIG. 1E). To test whether UFD-2 has any influence on CHN-1 self-ubiquitylation ability, was compared in vitro self-ubiquitylation of CHN-1 in the absence and in the presence of excess amounts of UFD-2. There was no apparent difference in the efficiency of the reaction (FIG. 1E, bottom).

Accordingly, UFD-2 and CHN-1 act in the same conjugation pathway to degrade at least one specific substrate.

### EXAMPLE 3

### A chn-1 Loss of Function Mutant is Linked to Stress Tolerance

In order to gain further insight into the in vivo function of chn-1, a deletion mutant of chn-1 after screening a C. elegans deletion library was isolated. The mutation of the allele by155 is a 0.9-kilobase deletion that removes the 3′ splice site of exon 4 as well as the complete exons 5 and 6 (FIG. 2A). Northern blot analysis showed that the chn-1 gene in chn-1(by155) mutant animals is transcribed, as expected, as a shorter message with significantly reduced amounts compared to wild-type. The transcription efficiency of the downstream gene T09B4.9, belonging to the same operon (Blumenthal, 2002), is not affected by this deletion (FIG. 2B). If expressed in vivo, the corresponding protein would be C-terminally truncated, consisting of the N-terminus leaving only two of the three TPR motifs intact (CHN-1^{Δ}) (FIG. 2C). Such a CHN-1 version without the U-box is expected to no longer act as an ubiquitylation enzyme (FIG. 1E, bottom). Different truncated versions of CHN-1 (FIG. 2C) were bacterially expressed and it was found that CHN-1^{Δ} is not able to interact with GST-UFD-2, unlike CHN-1 that lacks the U-box (CHN-1^{ΔU-box}), which is still able to bind (FIG. 2D). These results show that, if translated, the gene product of chn-1(by155) is unable to interact with UFD-2. Furthermore, CHN-1^{Δ} is also not able to interact with the C. elegans Hsp70 homolog HSP-1 (FIG. 2E).

The chn-1(by155) mutant is viable and displays no obvious morphological defects. No significant alterations in motility, egg-laying behavior or life span were identified under normal growth conditions but a reduced brood size (250±6 progenies (n=18) in contrast to 297±5 progenies (n=18) for N2 wild-type at 20° C.) was observed. Shifting chn-1 mutants to temperatures greater than 30° C. before they produce eggs led to developmental arrest and lethality at different larval stages in the F1 generation (data not shown). chn-1 (RNAi) treated wild-type nematodes displayed similar temperature sensitivity (C. Holmberg and R. Morimoto, personal communication).

A strain trans-heterozygous for chn-1(by155) and chn-1(ok459) was generated, which represents a loss-of-function allele (isolated by the C. elegans Gene Knockout Consortium, Oklahoma) (FIG. 2A). 7/8 chn-1(by155)/chn-1(ok459) trans-heterozygotes behaved like chn-1(by155) homozygotes. This supports our conclusion that by155 is a complete loss-of-function allele. Since chn-1(ok459) homozygous mutants are not viable due to deletions of neighbouring genes chn-1(by155) were used for further studies.

The assembly of the motor protein myosin into the contractile ring during cytokinesis and into thick filaments during muscle development are still largely unexplored processes. Recent results suggest that myosins do not assemble autonomously but rather need additional factors (Srikakulam, 2004). One likely candidate for this process is the Hsp90 co-chaperone UNC-45, which itself exerts chaperone activity on muscle myosin II during thick filament assembly and interacts with non-muscle myosin II and unconventional myosin V (Barral, 2002; Hutagalung, 2002).

In context of this invention, it was found that UNC-45 is ubiquitylated in vivo and that the CHIP ortholog CHN-1 together with UFD-2 are necessary and sufficient for its multiubiquitylation in vitro. The ‘bag of worms’ phenotype and the movement defect of unc-45 (ts) mutants can be suppressed in animals lacking CHN-1 most likely due to stabilization of the corresponding UNC-45 (ts) proteins. Importantly, this suppression needs residual UNC-45 activity. However, excessive amounts of UNC-45 appear to be toxic for myosin assembly in the chn-1(by155) mutant. The E2 enzyme LET-70 (Jones, 2002) specifically collaborates with CHN-1 and UFD-2 in self-ubiquitylation assays as well as in UNC-45 conjugation. Thus, this E2 enzyme is considered to function in this pathway in vivo. Supporting the results, Jones has shown that arrested larvae resulting from let-70 (RNAi) treatment of wild-type worms have defects in muscle sarcomere assembly (Jones, 2002). Down regulation of the proteasomal subunit RPT-2 by RNAi leads to a similar suppression of unc-45 (ts) mutants as shown by chn-1 loss-of-function. Thus, the protein levels of the myosin chaperone UNC-45 necessitate stringent regulation, which appears to be dependent on CHN-1 and UFD-2 ubiquitylation activity (FIG. 7).

### EXAMPLE 4

### CHN-1 and UFD-2 Interact with the Myosin Chaperone UNC-45

It was found that purified FLAG-epitope-tagged UNC-45 (^{FLAG}UNC-45) interacted with purified GST-UFD-2 (FIG. 3A). UNC-45 has been recently shown to stoichiometrically bind the chaperone Hsp90 and myosin (Barral, 2002). In several pull-down assays carried out in context of this invention, Hsp90 did not compete for the interaction between UFD-2 and UNC-45.

Then it was tested whether CHN-1 was also able to interact with UNC-45 and it was found that a GST-tagged version of CHN-1 bound purified UNC-45. To compare their binding efficiencies, pull-down experiments were performed using equimolar amounts of GST-UFD-2 or GST-CHN-1 with identical amounts of purified UNC-45. GST-UFD-2 bound-20-fold more UNC-45 when compared to GST-CHN-1 (FIG. 3B). Next, it was examined whether there was any competition between CHN-1 and UNC-45 for UFD-2 binding. Binary mixtures containing immobilized UFD-2 and either UNC-45 or CHN-1, and a ternary mixture containing all three proteins were set up and the resulting complexes analyzed by SDS-polyacrylamide gel electrophoresis. Approximately equimolar complexes were detected between UFD-2 and both CHN-1 and UNC-45 in the binary mixtures and among the three proteins in the ternary mixture. There was no significant change in binding of either UNC-45 or CHN-1 to UFD-2 in the ternary mixture in comparison to the binary mixtures (FIG. 3C). UFD-2 is, accordingly able to bind CHN-1 and UNC-45 simultaneously.

After it was shown that UFD-2, CHN-1 and UNC-45 can interact in vitro, it was asked whether their tissue distribution overlaps in vivo. The expression pattern of CHN-1 and UFD-2 was analyzed in wild-type animals transgenic for constructs that encode each protein fused with green fluorescent protein (GFP) under the control of the endogenous promoters. Both GFP::CHN-1 and UFD-2::GFP fusion proteins displayed overlapping expression throughout the transgenic animals from the early embryo to adulthood in a variety of tissues including body wall muscle cells, neurons and hypodermis, but were absent from the intestine (FIG. 3D-H). UNC-45 is expressed in body wall muscle cells throughout development as well (Ao, 2000; Venolia, 1999). Importantly, the P_{chn-1}::GFP::chn-1 construct used for expression analyses is able to rescue chn-1(by155) (FIG. 4A). Therefore it was concluded that, at least in body wall muscle cells, the protein interactions observed in vitro can occur in vivo as well.

### EXAMPLE 5

### chn-1(by155) Specifically Suppresses Different unc-45 Temperature-Sensitive Mutants

In order to study the putative interaction between CHN-1 and UNC-45 in vivo, chn-1(by155) were crossed with two temperature-sensitive (ts) unc-45 mutants, unc-45(e286) and unc-45(m94), which encode full-length proteins with a single amino acid substitution each (Barral, 1998). When grown at the restrictive temperature for unc-45 (ts) alleles (20° C. or above), homozygous animals in each case are fully paralyzed due to disorganized myofilament arrays in the body wall muscles (as shown in FIG. 5C). In contrast, growth at the permissive temperature of 15° C. leads to an essentially wild-type phenotype. Temperature shifts reverse the unc-45 (ts) phenotype in developing embryos or larvae, when most myofilament assembly occurs, but not in adults (Epstein, 1974). In addition, uncoordinated worms, including unc-45 (ts) mutants retain an excessive amount of eggs in the uterus (an egg-laying defective or EgI phenotype). These eggs hatch and develop inside the mother, leading to a terminal ‘bag of worms’ (Bag) phenotype (Waterston, 1988). The chn-1 loss-of-function mutation by155 suppresses significantly, but not completely, the ‘bag of worms’ phenotype of unc-45(e286) and unc-45(m94) after shifting L3 larvae from 15° C. to 22° C. (FIG. 4A). The egg-laying defect of unc-45 (ts) mutants severely limits the number of progeny generated. chn-1(by155); unc-45(e286) and chn-1(by155); unc-45(m94) double mutants display a significantly enhanced brood size in contrast to the single mutants, but still not equivalent to the wild-type (FIG. 4B). The specificity of this genetic interaction was demonstrated with the restoration of the ‘bag of worms’ phenotype to unc-45 (ts) mutant behavior by the expression of a functional copy of chn-1 from a genomic subclone (identical to the one used for the expression studies in FIG. 3D-F) or the chn-1 cDNA under the muscle specific control of the unc-54 promoter (FIG. 4A). This transgenic rescue indicates that the suppression of temperature-sensitive unc-45 alleles is due to the deletion in the chn-1 gene. Furthermore, expression of each transgene alone on a wild-type background shows no effect on muscle structure (data not shown). In order to detect subtle movement defects, temperature shifted worms were allowed to crawl for one hour on agar plates completely seeded with a lawn of E. coli cells and the traces were then photographed (FIG. 4C). The double mutants chn-1(by155); unc-45(e286) and chn-1(by155); unc-45(m94) displayed significantly better movement than unc-45(e286) or unc-45(m94) alone.

Complete loss-of-function alleles of unc-45 result in embryonic lethality, which demonstrates the requirement of unc-45 function for normal embryonic development in C. elegans (Venolia, 1990). Other UCS-domain proteins have been demonstrated to be required for proper cytokinesis (S. pombe Rng3p and human general cell-type UNC-45) (Price, 2002; Wong, 2000). This is consistent with a localization of UNC-45 at the cleavage furrow in the early embryo. Moreover, recent two-hybrid analysis suggests that, in addition to the muscle myosins, cytoskeletal type II and unconventional type V myosins interact with the UNC-45 protein (Hutagalung, 2002). However, in contrast to the suppression of unc-45 (ts) mutants, chn-1(by155) is not able to suppress the embryonically lethal allele unc-45(st604) (Venolia, 1990) (25/25 chn-1(by155); unc-45(st604) embryos arrested like the unc-45(st604) single mutant at the twofold stage of embryonic development). It was asked whether the chn-1 mutation suppresses specifically unc-45 alleles or, rather more generally, mutations in body wall muscle components. Therefore double mutants of chn-1 and unc-54, the gene encoding body wall muscle myosin B (Epstein, 1974) were analyzed. Loss of chn-1 does not suppress the EgI phenotype of the temperature-sensitive mutant unc-54(e1301) (29/30 chn-1(by155) unc-54(e1301) double mutants were still EgI), indicating that suppression is indeed specific for unc-45 (ts) alleles.

These findings show that some defects of the unc-45 (ts) phenotype can be suppressed specifically by loss-of-chn-1-function. However, since this suppression needs the residual UNC-45 activity provided in unc-45 (ts) animals, these findings dokument that CHN-1 acts as a negative regulator of UNC-45.

CHIP was first identified as a co-chaperone of Hsc70 and Hsp90 (Ballinger, 1999). In recent studies it was found that the C-terminal U-box domain of CHIP has ubiquitin ligase activity (Jiang, 2001). Thus, CHIP may acts as a protein quality-control ubiquitin ligase that selectively leads abnormal proteins recognized by molecular chaperones to degradation by the 26S proteasome. Although accumulating evidence from in vitro studies suggests that this may be the case, actual biological pathways involving CHIP have remained largely unexplored (Murata, 2003).

CHN-1 displays U-box-dependent self-ubiquitylation activity and that it interacts with C. elegans Hsp70, consistent with previous results involving CHIP (Ballinger, 1999; Jiang, 2001). CHN-1 is a bona fide C. elegans ortholog of CHIP. Surprisingly, in contrast to the speculated key role for CHIP in modulating the cellular response to stress, CHN-1, encoded by a single gene in C. elegans, is not essential for viability. The ability of CHIP/CHN-1 to mediate ubiquitylation and its co-chaperone activity does not represent a link between abnormal protein degradation and protein folding in vivo. UFD2 is also required for stress tolerance in yeast cells deficient for the proteasomal subunit RPN10 (Koegl, 1999), which suggests that U-box proteins are particularly well suited to function under conditions of cellular stress.

Here in vitro and in vivo data were presented, that document the direct role of CHN-1 in the regulation of myosin assembly by ubiquitylation of the myosin chaperone UNC-45 (FIG. 7). Therefore, UNC-45 represents the first natural substrate identified for the CHIP ortholog CHN-1.

### EXAMPLE 6

### Muscle Specific Expression of unc-45 Leads to Myosin Assembly Defects in the chn-1(by155) Mutant

To assess the effect of unc-45 overexpression on a chn-1-deficient background, the unc-45 cDNA from an extra chromosomal array was expressed under the control of the muscle specific unc-54 promoter in unc-45(m94), chn-1(by155) and N2 wild-type worms. Importantly, expression of this transgene (P_{unc-54}::unc-45) rescues the ts phenotype of unc-45(m94) at the restrictive temperature of 25° C., as assessed by both improved motility and muscle structure, demonstrating that it expresses functional levels of UNC-45 protein (FIG. 5A-C). The worms were allowed to crawl for one hour on agar plates completely seeded with a lawn of E. coli cells and the traces were then photographed (FIG. 5A). Additionally, it was determined how many body bends per minute the worms can perform in liquid medium, a reliable way of assessing subtle contractile defects (FIG. 5B). It was found that wild-type and unc-45(m94) worms expressing functional UNC-45 behave essentially like untransformed wild-type worms. However, extra copies of unc-45 in the chn-1(by155) mutant lead to a dramatic Unc phenotype, resulting in animals that are fully paralyzed.

Next it was sought to study the defects in muscle structure of these strains by polarized light microscopic examination. Wild-type worms have body wall muscle cells exhibiting periodic sarcomeric structures such as A bands, I bands, dense bodies, and H zones (Waterston, 1988). In line with the movement data, the transgenic expression of unc-45 did not affect muscle structure in wild-type and rescued the muscular defects of the unc-45(m94) allele, which normally displays a disorganized sarcomeric structure. This analysis also showed that the muscle structure of chn-1(by155) is comparable to that of wild-type, but the expression of transgenic unc-45 led to strong sarcomeric assembly defects (FIG. 5C). Expression of UNC-45 protein from an integrated transgene (produced from the extra chromosomal array Ex[P_{unc-54}::unc-45^{FLAG}]) was significantly less than that from the extra chromosomal array (˜20-fold less) and did not lead to any detectable sarcomeric defects in the chn-1(by155) mutant background (data not shown). Therefore, the amount of UNC-45 protein present in the muscle cells is critical for proper function in worms lacking CHN-1.

Taken together, the results demonstrate that chn-1 loss-of-function not only suppresses temperature-sensitive unc-45 alleles, but is also not able to compensate for excessive amounts of UNC-45.

### EXAMPLE 7

### UNC-45 is a Substrate for CHN-1 and UFD-2 Dependent Multiubiguitylation

The direct physical interaction between CHN-1 and UNC-45, the chn-1(by155)-dependent suppression of unc-45 (ts) mutants, and the myosin assembly defects resulting from extra chromosomal expression of unc-45 in the chn-1(by155) mutant background suggest that UNC-45 might be a substrate for CHN-1-dependent ubiquitylation. To examine this possibility, in vitro ubiquitylation assays were performed using recombinantly expressed E1, the E2 enzyme LET-70 (DeRenzo, 2003; Jones, 2002) and CHN-1. These enzymes were incubated together with the purified FLAG-tagged UNC-45 protein in a buffer containing ATP and ubiquitin. As shown in FIG. 6A (left panel), the activities of all three enzymes together are required and sufficient for the recognition and ubiquitylation of UNC-45 in vitro. In contrast to LET-70 alone the addition of full-length CHN-1 proceeds to efficient ubiquitin conjugation. However, the majority of conjugates appeared to contain only one to three ubiquitin moieties. As expected from the self-ubiquitylation studies, a CHN-1 version lacking the U-box, CHN-1^{ΔU-box}, was not able to ubiquitylate UNC-45 (FIG. 6B).

A similar reaction containing UFD-2 instead of CHN-1 led to a corresponding result: addition of one to three ubiquitins to the substrate protein UNC-45 (FIG. 6A, right panel). Thus, CHN-1 and UFD-2 have a limited capacity to multiubiquitylate UNC-45 in vitro. Next it was tested whether incubation of UNC-45 with both CHN-1 and UFD-2 simultaneously during the reaction would give a different result. It was found that these enzymes together dramatically stimulated the multiubiquitylation reaction, yielding UNC-45 conjugates with significantly longer chains than those conjugated by CHN-1 or UFD-2 alone (FIG. 6C).

To determine whether UNC-45 is in fact ubiquitylated in vivo, wild-type worms were examined expressing functional FLAG-tagged UNC-45 (the extra chromosomal array Ex[P_{unc-54}::unc-45^{FLAG}] rescues the myosin assembly defect and thereby the movement disorder of unc-45(m94) (see FIG. 5A-C)). Indeed, immunoblot analysis of protein samples obtained by immunoprecipitation of UNC-45 revealed ubiquitin-immunoreactive proteins with sizes slightly larger than full-length UNC-45. Furthermore, immunoprecipitated UNC-45 recombinantly expressed and purified from baculovirus-infected insect cells was not ubiquitylated (FIG. 6D). In summary it was concluded from these data that CHN-1 and UFD-2 form a complex that directly multiubiquitylates the myosin chaperone UNC-45 in vivo.

E3 enzymatic activity is usually sufficient for multiubiquitylation of substrate proteins. However, some E3 enzymes need additional factors. For example, the tumor suppressor protein BRCA1 is only active as a functional E3 ligase when in a heterodimeric E3-like complex with BARD1 (Hashizume, 2001). Yeast UFD2 has been recently identified to act as a multiubiquitylation factor, which binds to the ubiquitin moieties of preformed conjugates and catalyzes multiubiquitin chain assembly in conjunction with E1, E2, and E3, an activity designated as E4 (Koegl, 1999). A similar E4-like function has been identified by Imai and co-workers for mammalian CHIP (Imai, 2002).

This work revealed an unexpected aspect of multiubiquitylation pathways. The regulation of UNC-45 described here appears to involve a novel E4 activity linked to a heterooligomeric complex of two E3 enzymes. In contrast to in vitro results with yeast UFD2 (Koegl et al., 1999), the C. elegans ortholog UFD-2 is able to add one to three ubiquitin molecules in the absence of an additional E3 enzyme to its substrate UNC-45 (FIG. 6A, right panel). A similar reaction occurs when only CHN-1 is used (FIG. 6A, left panel). Therefore, in C. elegans both CHN-1 and UFD-2 work independently as E3 enzymes in this ubiquitylation pathway. Remarkably, it was found that only combination of the E3 enzymes CHN-1 and UFD-2 leads to appropriate multiubiquitylation of the substrate UNC-45 (FIG. 6C). Thus, a situation has been identified in which two E3 activities in combination lead to formation of a complex with E4-like activity (FIG. 7).

Such an E3/E4 complex offers a variety of possibilities for regulation of cellular pathways. For example, the presence of a regulated assembly factor during the cell cycle could allow different degradation times for different substrates of the same multiubiquitylation complex. In multicellular organisms like C. elegans, substrate degradation could be induced by tissue specific co-expression of both E3 enzymes in a developmentally regulated manner. It is not unreasonable to propose that different combinations of E3 enzymes could lead to alternative complexes that mediate E4 activity. Interestingly, CHIP interacts with the E3 enzyme Parkin (Imai, 2002) and Nikolay has recently shown that CHIP is also able to form a homodimer (Nikolay, 2004).

Several CHIP dependent substrates have been identified from experiments with CHIP overexpressing cells or from in vitro reconstitution systems. Surprisingly, some of these substrates are not efficiently multiubiquitylated in vitro (ErbB2, raf-1 kinase) (Demand, 2001; Xu, 2002) and ubiquitylation of some substrates is not sufficient for degradation (Hsc70, BAG-1) (Alberti, 2002; Jiang, 2001). It is thus conceivable that a similarly conserved CHIP/UFD2 complex to the one described here is indeed necessary to efficiently multiubiquitylate a variety of substrates in vivo.

UNC-45 homologs have been identified in Drosophila, Xenopus, zebrafish, mouse, and human. They all share the same domain configuration found in C. elegans UNC-45: an N-terminal TPR domain, a unique central region and a C-terminal UCS domain (Barral, 2002; Hutagalung, 2002). Significantly, mutated residues in C. elegans and S. pombe UCS domains are identical or conserved throughout all identified homologs (Barral, 2002). The UNC-45 homolog from zebrafish was recently shown to be required for muscle thick fiber assembly upon interaction with myosin (Etheridge, 2002). Both human and mouse genomes contain two isoforms of UNC-45, which have separate, but possibly overlapping functions in striated muscle differentiation (Price, 2002). Thus, the function of UNC-45 in myosin assembly is highly conserved among invertebrate and vertebrate species.

CHN-1 and UFD-2 also have homologs from yeast to man, including C. elegans. Expression studies of human CHIP showed that it is highly expressed in adult human striated muscles (skeletal muscles and heart) as well as in a developmentally and spatially regulated fashion in the mouse embryo, particularly during the course of cardiac and skeletal myogenesis (Ballinger, 1999). However, the functional significance of this tissue-specific expression pattern of CHIP is presently unclear. A regulatory function of the CHIP ortholog CHN-1 was shown in myosin assembly in the nematode C. elegans, which could provide insight into similar roles for CHIP during mammalian development. A similarly conserved CHIP/UFD2/UNC45 mammalian complex probably mediates an equivalent ubiquitin-dependent regulation on processes that require myosin assembly. Like CHIP, human and mouse UFD2 are highly expressed in skeletal muscle (Ballinger, 1999; Kaneko, 2003; Mahoney, 2002). Both the mouse homologs UFD2a, UFD2b and CHIP collaborate with the same E2 enzymes Ubc4 and UbcH5c (Hatakeyama, 2001; Jiang, 2001; Murata, 2001). The proteasome subunit S5a interacts physically with CHIP (Connell, 2001) and the yeast S5a homolog RPN-10 interacts epistatically with UFD2 in S. cerevisiae (Koegl, 1999).

### EXAMPLE 8

### The Influence of CHIP on Muscle Degeneration

CHIP acts as a linker and regulator between the chaperone system, which detects and repairs damaged proteins, and the degradation machinery of the ubiquitin-proteasome system (Koegl, 1999; Connell, 2001).

The C. elegans muscle dystrophy mutant carries mutations in the dystrophin gene dys-1(cx18) and in the homologe of the muscle-specific helix-loop-helix transcription factor MyoD hlh-1(cc561). The animals show a time dependent loss of mobility and egg-laying, and their muscles degenerate when they reach adulthood (Gieseler, 2000).

To test whether downregulation of the CHIP ubiquitin ligase activity has an influence on muscle degeneration, dys-1(cx18);hlh-1(cc561) C. elegans were fed with chn-1(RNAi) bacteria. The bacteria expressing dsRNA of chn-1 (T09B4.10) were achieved from the C. elegans RNAi library of J. Ahringer's laboratory. chn-1(RNAi) increased the locomotion rate of dys-1(cx18);hlh-1(cc561) worms up to two to three fold but had no detectable effect on the locomotion rate of the individual single mutants dys-1(cx18) and hlh-1(cc561). Accordingly, CHIP has an influence on the degradation of muscle cells in dys-1(cx18);hlh-1(cc561) worms. A possible explanation for the effect was a shift from the protein degradation via the ubiquitin-proteasome pathway to an increased protein reparation by chaperones. CHIP was thought to be responsible for this shift: heat shock proteins like Hsc70 are common targets, that are ubiquitylated by CHIP (Jiang, 2001). The activity of ubiquitylated chaperones is strongly reduced and following less misfolded protein is repaired. When the activity of CHIP is reduced or the protein is missing, the ubiquitylation of chaperones is downregulated and the level of protein repair is increased in the cell. Un- or misfolded proteins of the DGC will be folded correctly, and the DGC reformation will lead to a higher stability of the muscles, which allows an increased mobility of the individuum.

The mobility of the dys-1(cx18);hlh-1(cc561) was even dramatically improved when the animals were crossed with chn-1 mutants. The allele by155 and a by155/ok459 heterozygous animals were used for these experiments. The two chn-1 alleles ok459 and by155 by the C. elegans Knock out consortium and also described in Hoppe et al., Cell Aug. 6, 2004. Eliminating chn-1 activity in the by155 homozygous mutant or in the by155/ok459 heterozygous strain resulted in restoration of wild-type behaviour of the dys-1 (cx18); hlh-1 (cc561) double mutant.

When wild-type worms were tested in the locomotion assay (n=287) more than 95% of the animals had reached the bacteria after one hour. After the same time period 94±4% of chn-1(ok454/by155); dys-1(cx18);hlh-1(cc561) and 79±2% of chn-1(by155)dys-1(cx18);hlh-1(cc561), but only 10±6% of the dys-1(cx18);hlh-1(cc561) had reached the bacteria. The better mobility of the triple mutants was clearly due to the reduced muscle degeneration. dys-1(cx18);hlh-1(cc561) animals are hypercontracted, uncoordinated (unc) and egg-laying defective (egl), all results of the increasing muscle degeneration when they reach adulthood. These phenotypic characteristics are strongly reduced in the chn-1(ok459/by155)dys-1(cx18;hlh-1(cc561) worms, and chn-1(by155)dys-1(cx18); hlh-1(cc561) show wild-type body shape and number of eggs in the uterus again (FIG. 8).

The muscle degeneration in dys-1(cx18);hlh-1(cc561) C. elegans was until now only partially decreased through overexpression of two members of the dystrophin-glycoprotein complex. dyc-1 (similar to neutral nitric oxide synthase nNOS-binding protein CAPON) overexpression partially supressed the dys-1(cx18);hlh-1(cc561) phenotype (Gieseler, 2000), and dyb-1 (dystrobrevin-like) overexpression delayed the outcome of the dys-1(cx18);hlh-1(cc561) phenotype for one day (Gieseler, 2002). A reduced muscle degeneration as detected, was only found in dys-1(cx18);hlh-1(cc561) with RNAi-mediated inhibition of the egl-19 calcium channel (Mariol, 2001) The reduced calcium levels in the cells due to reduced calcium channel activity lead to much slower degeneration of the muscle cells.

The inhibition of the ubiquitin-proteasome pathway is a way of reducing the muscle degeneration in DMD patients. CHIP is a single muscle specific protein that plays a keyrole in described process and therefore, CHIP is a key player in new therapy strategies against muscular disease, in particular dystrophies and most preferably Duchenne Muscle Dystrophy.

### EXAMPLE 9

### siRNA Transfection of Myoblasts and Quantitative Real-Time PCR

Human myoblasts from the biopsy samples were selected according to an established procedure of the Muscle Tissue Culture Collection (Munich, Germany). To obtain primary human myoblast cultures muscle tissue samples of neuromuscular patients were treated proteolytically. After 2-3 weeks in culture 2×10⁷ myoblasts were cryopreserved for each sample. Pure myoblast cultures were obtained by magnetically activated cell sorting using an antibody to neural cell adhesion molecule, as previously described.

Human or mouse myoblasts were maintained in SkMC growth medium (PromoCell, Heidelberg, Germany), at 37° C., 5% CO2, and 95% humidity prior to transfection.

Cells were plated at a density of 60% in six-well culture dishes on the day before transfection. Immediately before transfection the cells were washed with phosphate-buffered saline, and 1 ml SkMC growth medium without serum was added. Transfection was carried out using Genesilencer (PeqLab, Erlangen, Germany) according to the manufacturers protocol. Briefly, 5 μl Genesilencer reagent was mixed with 25 μl serum-free medium. 1000 ng siRNA was diluted in a mix of 25 μl siRNA diluent and 15 μl serum-free medium and incubated for 5 min at room temperature. siRNA solution was added to the diluted Genesilencer and incubated for 5 min at room temperature before adding to the cells. Serum was omitted in the medium for the first 4 h after transfection. Then 1 vol medium with a serum concentration of 10% was added to a final concentration of 5%. Twenty-four hours after transfection the medium was changed to SkMC differentiation medium (Promocell) with 2.5% fetal bovine serum (for myoblasts) or changed to DMEM with 2% horse serum (for differentiation of myoblasts into myotubes).

SiRNAs (SEQ ID Nos: 13 to 18 for human and SQ ID NOs: 19 to 24 for mouse) have been synthesized via the siMAX online service of MWG BIOTECH (Ebersberg, Germany) as standard 21mers comprising (rUrU)-3′ or (dTdT)-3′ overhangs. Note that the siRNAs provided in any one of SEQ ID NOs: 13 to 24 comprised in this experimental setting (or preferably also in a medical setting) further modifications, like the herein mentioned additional (stabilizing) rUrU/dTdT overhangs. Examples of siRNAs are also illustrated in FIG. 9.

After 72 h total incubation, RNA form ca. 5×10³ myoblasts/myotubes was isolated according to manufacturers recommendations (Quiagen RNeasy Kit) or Chomczynski and Sacchi (Anal Biochem. 1987; 162:156-159).

The pellets were air-dried and resuspended in Nuclease-free water (Promega, Inc.) to a final concentration of 0.2 μg/μl. 1 μg RNA each was dissolved in 25 μl DNase digestion solution (2.5 μl 10×DNasel digestion buffer, 2 μl DNase I (10 IU/μl; Roche, Mannheim, Germany), and incubated at 37° C. for 30 min to remove genomic DNA contamination. After enzyme inactivation at 75° C. for 10 min, the solution was stored at −80° C. The absence of genomic DNA was controlled by PCR using isolated RNA as positive control template.

For mRNA quantification, 12.5 μl RNA samples were incubated with 100 ng random hexamer primer (pN6; Roche) for 5 min at 70° C., and chilled on ice. RT was performed in a total volume of 20 μl containing 50 mM Tris-HCl (pH8.3); 75 mM KCl; 3 mM MgCl₂; 10 mM dithiothreitol; 1 mM each of deoxy (d)-GTP, dATP, dTTP, and, dCTP (MBI Fermentas, St. Leon-Rot, Germany); and 200 IU murine leukemia virus reverse transcriptase (high specific activity; GIBCO Life Technologies, Inc.). The RT reaction was carried out at 25° C. for 10 min, then at 42° C. for 1 h, terminated by 10 min at 70° C., and stored at −80° C.

Real-time PCR was carried out using the TaqMan Universal Master Mix and MicroAmp Optical plates and caps (Applied Biosystems, Foster City, Calif., USA) as described in: Jaksch et al. (2001) Hum Mol. Genet. 10: 3025-3035.

Quantification of mRNA abundance was performed by real-time PCR detection using an ABI PRISM 7700 sequence detector (PE Biosystems, Inc., Weiterstadt, Germany) and SybrGreen as a double stranded DNA-specific fluorescent dye. Amplification mixes (25 μl) contained 2.5 μl complementary DNA (cDNA); 12.5 μl 5×SybrGreen PCR buffer; 200 μM dATP, dCTP, dGTP, and 400 μM dUTP; 3 mM MgCl₂; 300 nM (=7.5 pmol) of each primer; 0.25 IU AmpErase uracil N-glycosylase; and 0.625 IU AmpliTaq Gold DNA polymerase (PE Biosystems, Inc.). Amplification primers were synthesized according to primer pairs listed in the PrimerBank (Xiaowei Wang and Brian Seed (2003) A PCR primer bank for quantitative gene expression analysis. Nucleic Acids Research 31: e154. p 1-8.), which can be accessed through: http://pga.mgh.harvard.edu/primerbank/index.html. Primer sequences can be retrieved via PrimerBank Id numbers. The following primer pairs for quantification of human CHIP-cDNA (NM_{—}005861; shown in SEQ ID NO:3) have been used:

| | |
|---|---|
| PrimerBank Id 5031963a1: | |
| 5′-AGCAGGGCAATCGTCTGTTC | (SEQ ID NO:25) |
| and | |
| 5′-CATCTTCAGGTAGCACAAGGC | (SEQ ID NO:26) |
| PrimerBank Id 5031963a2: | |
| 5′-TGCACTCCTACCTCTCCAGG | (SEQ ID NO:27) |
| and | |
| 5′-TTGTCGTGCTTGGCCTCAAT | (SEQ ID NO:28) |
| PrimerBank Id 5031963a3: | |
| 5′-GCGGACATGGACGAGCTTT | (SEQ ID NO:29) |
| and | |
| 5′-CCTTGCGGTCGTAGGTGATG | (SEQ ID NO:30) |

The following primer pairs for quantification of murine CHIP-cDNA (AK002752; shown in SEQ ID NO:7) have been used:

| | |
|---|---|
| PrimerBank Id 9789907a1: | |
| 5′-CGGCAGCCCTGATAAGAGC | (SEQ ID NO: 31) |
| and | |
| 5′-CACAAGTGGGTTCCGAGTGAT | (SEQ ID NO: 32) |
| PrimerBank Id 9789907a2: | |
| 5′-CCACTTGTGGCAGTGTACTAC | (SEQ ID NO: 33) |
| and | |
| 5′-TGGCCTCATCATAACTCTCCAT | (SEQ ID NO: 34) |
| PrimerBank Id 9789907a3: | |
| 5′-AAGGAGCAGCGACTCAACTTT | (SEQ ID NO: 35) |
| and | |
| 5′-CAGCAGCAATGAGCCTGGT-3′ | (SEQ ID NO: 36) |

The primer pair: 5′-ACCCCAGCCATGTACGTAGC (SEQ ID NO: 37) and 5′-GTGTGGGTGACCCCGTCTC (SEQ ID NO: 38) were used for quantification of human and murine β-Actin-cDNA (as described in: Jaksch et al. (2001) Hum Mol. Genet. 10: 3025-3035).

PCR was started with 2 min at 50° C. for AmpErase activation and 10 min at 95° C. for denaturation. The program continued with 40 cycles of 15 sec at 95° C. and 60 sec at 60° C. Each assay included triplicates of cDNA primed separately for the gene of interest (i.e. CHIP), for the reference gene β-Actin, a no template control, and four dilutions of cDNA (1:25, 1:50, 1:100, and 1:200) to calculate the corresponding amplification efficiency (E=10^{-(1/b)}−1; b=regression coefficient). The parameter C_{T} (threshold cycle) is defined as the cycle number at which fluorescence intensity exceeds a fixed threshold. Relative mRNA expression for each gene of interest (I) was calculated using the formula: (1+E_{(I)})^{-CT(I)}/(1+E_{(β-Actin))}^{-CT(β-Actin)}. These values obtained for the myoblasts/myotubes treated with siRNAs were referenced to the corresponding values obtained for untreated cells and healthy control myoblasts/myotubes, respectively.

### Downregulation of CHIP Expression in Human Myoblasts by Hs-CHIP-siRNAs

TABLE
C_{T} values for CHIP- and β-Actin-mRNA

| | Myoblasts | +Hs-CHIP- | +Hs-CHIP- |
|---|---|---|---|
| | (DMD patients) | siRNA 1 | siRNA 2 |
| CHIP | 28.1 | 29.4 | 29.2 |
| β-Actin | 25.7 | 25.5 | 25.9 |
| CHIP/ | 1.09 | 1.15 | 1.13 |
| β-Actin | | | |

In accordance with this experimental setting, a down-regulation of CHIP-mRNA could be obtained which corresponds to 35 to 50% of the CHIP-mRNA levels of untreated cells, i.e. a reduction of expression of 50 to 65%.

The siRNA treated myoblasts and myotubes are analyzed by Western Blot and immunostaining for presence of dystro- and sarcoglycans. For DMD myotubes, where a (secondary) reduction of adhalin or sarcoglycans has been confirmed by Western Blot analysis, an increase of these DAG components in the siRNA treated cells is envisaged, being a clear clinical improvement.

Macromolecular improvements such as stability of cellular membranes of siRNA treated myoblasts and myotubes can be verified by the Evans Blue test.

Sequences of nucleic acid molecules for CHN-1/CHIP
orthologs and correspondinq amino acid sequences
(SEQ ID NOs: 1 to 12)

SEQ ID NO: 1 chn-1 genomic [Caenorhabditis
elegans] (GenBank ACCESSION NM_059380)
CGTTTGCAGCATGTCAAGCGGCGCCGAACAACATAATACTAATGGCAAAA
AGTGTTATATGAATAAAAGATATGACGATGCCGTTGATCACTATTCGAAA
GCCATCAAAGTAAATCCTCTGCCGAAATACTATCAGAACAGAGCAATGTG
TTACTTTCAGCTTAACAACCTCAAGATGACTGAAGAAGACTGCAAACGGG
CACTGGAACTGAGCCCAAACGAAGTGAAACCTTTATACTTCTTGGGAAAT
GTATTTCTACAGAGCAAAAAGTACAGTGAAGCAATAAGCTGTCTCTCCAA
AGCGCTCTACCATAATGCTGTTATCACGAACGCTCCAGATATTGAGAACG
CCCTCAAACGGGCACGCCATCAAAAGTACGAGGAGGAGGAGTCAAAGCGA
ATTGTACAAGACGTTGAATTTCACACGTATTTGGAGAGCTTGATCGAAAA
GGATCGTCAAGAAAATTCTGAAAACCCAGAGGAGTTGCAAAGAGCAGATA
TGGCTAAAAAACGTCTAACCGAACTCACTTTAGCGACACAAGAAAAACGG
CAAAACAGGGAGGTTCCTGAGATGCTCTGCGGCAAAATTACACTGGAACT
GATGAAAGAGCCGGTGATAGTTCCATCTGGAATCACGTATGACCGAGAAG
AAATTGTGCAGCATTTGAGAAGAATTGGCCATTTCGATCCAGTCACAAGA
AAACCACTTACCGAAAATGAAATTATTCCGAATTATGCACTGAAAGAGGT
TATTGAAAAGTTTCTTGACGACAACCCATGGGCTAAATATGAACCCGGAG
GCATGGTTTAAATACTATTCAGAATATTGAAAAATTCAACATTAAACATT
CCTGGCTGCTTCTCAATGCAACTTCTTCACTTTTCTACATGTTTCAGTTC
TTTGCATTTTTTTTTCGGATTTTTAAACTTTTATTGTTTGAACGCTGTAC
CCTCCTTTTCTGTTCTACCGGTATTTGTATCTTGGTGTATTTTTTAGCTT
TAGTTTTCTTTCAAAATTCCATTGAATTTCTGGTAGAAAGTGCACTTCAA
CTTGTCGCTTAGTTCTCAATTTTACTGCTAAAAGTGAGATTCTGTAAATA
TTGTATGAAATAAAGAGGTGTGATGCTTTTC
SEQ ID NO: 2 C.elegans CHN-1 (Protein GenPept
Accession NP_491781)
MSSGAEQHNTNGKKCYMNKRYDDAVDHYSKAIKVNPLPKYYQNRAMCYFQ
LNNLKMTEEDCKRALELSPNEVKPLYFLGNVFLQSKKYSEAISCLSKALY
HNAVITNAPDIENALKRARHQKYEEEESKRIVQDVEFHTYLESLIEKDRQ
ENSENPEELQRADMAKKRLTELTLATQEKRQNREVPEMLCGKITLELMKE
PVIVPSGITYDREEIVQHLRRIGHFDPVTRKPLTENEIIPNYALKEVIEK
FLDDNPWAKYEPGGMV
SEQ ID NO: 3 human CHIP cDNA(STUB1) (GenBank
Accession NM_005861.1 or NM_005861.2)
CTGGGCCGCGAGGCGCGGAGCTTGGGAGCGGAGCCCAGGCCGTGCCGCGC
GGCGCCATGAAGGGCAAGGAGGAGAAGGAGGGCGGCGCACGGCTGGGCGC
TGGCGGCGGAAGCCCCGAGAAGAGCCCGAGCGCGCAGGAGCTCAAGGAGC
AGGGCAATCGTCTGTTCGTGGGCCGAAAGTACCCGGAGGCGGCGGCCTGC
TACGGCCGCGCGATCACCCGGAACCCGCTGGTGGCCGTGTATTACACCAA
CCGGGCCTTGTGCTACCTGAAGATGCAGCAGCACGAGCAGGCCCTGGCCG
ACTGCCGGCGCGCCCTGGAGCTGGACGGGCAGTCTGTGAAGGCGCACTTC
TTCCTGGGGCAGTGCCAGCTGGAGATGGAGAGCTATGATGAGGCCATCGC
CAATCTGCAGCGAGCTTACAGCCTGGCCAAGGAGCAGCGGCTGAACTTCG
GGGACGACATCCCCAGCGCTCTTCGAATCGCGAAGAAGAAGCGCTGGAAC
AGCATTGAGGAGCGGCGCATCCACCAGGAGAGCGAGCTGCACTCCTACCT
CTCCAGGCTCATTGCCGCGGAGCGTGAGAGGGAGCTGGAAGAGTGCCAGC
GAAACCACGAGGGTGATGAGGACGACAGCCACGTCCGGGCCCAGCAGGCC
TGCATTGAGGCCAAGCACGACAAGTACATGGCGGACATGGACGAGCTTTT
TTCTCAGGTGGATGAGAAGAGGAAGAAGCGAGACATCCCCGACTACCTGT
GTGGCAAGATCAGCTTTGAGCTGATGCGGGAGCCGTGCATCACGCCCAGT
GGCATCACCTACGACCGCAAGGACATCGAGGAGCACCTGCAGCGTGTGGG
TCATTTTGACCCGGTGACCGGGAGCCCCCTGACCCAGGAACAGTTCATCC
CCAACTTGGCTATGAAGGAGGTTATTGACGCATTCATCTCTGAGAATGGC
TGGGTGGAGGACTACTGAGGTTCCCTGCCCTACCTGGCGTCCTGGTCCAG
GGGAGCCCTGGGCAGAAGCCCCCGGCCCCTAAACATAGTTTATGTTTTTG
GCCACCCCGACCGCTTCCCCCAAGTTCTGCTGTTGGACTCTGGACTGTTT
CCCCTCTCAGCATCGCTTTTGCTGGGCCGTGATTGTCCCCTTTGTGGGCT
GGAAAAGCAGGTGAGGGTGGGCTGGGCTGAGGCCATTGCCGCCACTATCT
GTGTAATAAAATCCGTGAGCACGAAA
SEQ ID NO: 4 human CHIP protein sequence (GenPept
Accession AAD33400)
MKGKEEKEGGARLGAGGGSPEKSPSAQELKEQGNRLFVGRKYPEAAACYG
RAITRNPLVAVYYTNRALCYLKMQQHEQALADCRRALELDGQSVKAHFFL
GQCQLEMESYDEAIANLQRAYSLAKEQRLNFGDDIPSALRIAKKKRWNSI
EERRIHQESELHSYLSRLIAAERERELEECQRNHEGDEDDSHVRAQQACI
EAKHDKYMADMDELFSQVDEKRKKRDIPDYLCGKISFELMREPCITPSGI
TYDRKDIEEHLQRVGHFDPVTRSPLTQEQLIPNLAMKEVIDAFISENGWV
EDY
SEQ ID NO: 5 CHIP, Danio rerio (Sequence also
published in: Proc Natl Acad Sci U S A. 2002
Dec 24; 99(26):16899-903, GenPept Accession
AAH51775, GenBank Accession NM_199674)
ACCGAAACATGATGCTAGCGTCTCACACCCGCGGGTAAAATGTCGTGTTT
TTGAGGATCGCCGATGAAAACGTCGGACATTTGTCATCTTAATGTCATGA
TCGAGTCGGAATATGAGTTTGGCCGCGTTTCATTCGCTATCAGGGATCCA
AAAGTGATCATCAAAGCACCAGTGTCGCTACCGGTTTGCTAACGACAGGC
GTCAGTCCAGCAGTCAAACCCGAGGTTTGCAGGTAACTGGCTCCCCGGGA
CCGGCCGGTGCTCACCGAAGAGACGGGGAACAGGATTGGGAAGCAGGGGG
GAGCGGCCACCGCAGAGCCCATGGAGAAGATGGCGAGCAGCCCAGAGAAA
AGCTCATCCGCACAGGAGCTGAAGGAGCAGGGAAACCGCCTGTTCCTCAG
CCGCAAGTACCAGGAGGCCGTCACCTGCTACAGCAAAGCCATCAATCGTA
ACCCATCAGTGGCCGTGTACTACACTAACAGAGCGCTATGCTACGTCAAG
CTACAGCAGTACGACAAGGCACTGGCCGACTGTAAACACGCCCTTGAGCT
TGACAGCCAATCGGTGAAGGCGCACTTTTTTCTGGGCCAATGTCAGCTGG
AGCTGGAGAATTACGAAGAAGCCATCGGCAACCTACAGAGAGCCTATAAC
CTGGCGAAGGAACAGAGGCTCAATTTTGGAGACGACATCCCAAGTGCTCT
TCGCATCGCCAAGAAGAAACGCTGGAACAGCATAGAGGAGAAGCGGATCA
GCCAAGAGAACGAGCTGCACGCCTATCTCAGCAAACTCATCCTGGCCGAG
AAGGAGAGAGAGCTGGACGACCGAGTGAAGCAGTCAGATGACAGTCAGAA
TGGAGGAGACATCAGCAAGATGAAGTCCAAGCATGATAAGTATCTGATGG
ACATGGATGAGCTTTTCTCTCAAGTAGATGAAAAGAGGAAGAAGCGCGAG
ATCCCCGATTACCTGTGTGGGAAGATCAGCTTCGAGCTGATGAGGGAACC
CTGCATTACTCCCAGCGGCATCACCTACGACCGCAAGGACATCGAGGAGC
ATCTACAGCGTGTTGGCCATTTTGACCCCGTCACGCGTAGTCCTCTGACC
CAAGATCAGCTGATCCCCAACCTGGCCATGAAGGAGGTGATCGACGCTTT
CATCCAGGAGAACGGCTGGGTGGAGGACTACTGAAAACACACACACACAC
ACACTCTTCCTAATCACACACAATAATCACTCGCCTGCTGGTCTGGTACA
CACACACTTCCTCTCAATCTGCTGCATCCGATGATGATGATGATGATGCT
GTAGTTTCGCCTCTGAATGTTAAGAAGCAACATTATTGATGACACATCTC
CAATAATCATTGATAAAGAGGGAACTCTGTGCATGAGAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAA
SEQ ID NO: 6 CHIP protein sequence, Danio rerio
(GenPept Accession AAH51775)
MASSPEKSSSAQELKEQGNRLFLSRKYQEAVTCYSKAINRNPSVAVYYTN
RALCYVKLQQYDKALADCKHALELDSQSVKAHFFLGQCQLELENYEEAIG
NLQRAYNLAKEQRLNFGDDIPSALRIAKKKRWNSIEEKRISQENELHAYL
SKLILAEKERELDDRVKQSDDSQNGGDISKMKSKHDKYLMDMDELFSQVD
EKRKKREIPDYLCGKISFELMREPCITPSGITYDRKDIEEHLQRVGHFDP
VTRSPLTQDQLIPNLAMKEVIDAFIQENGWVEDY
SEQ ID NO: 7 mouse CHIP (GenBank Accession
AK002752)
GGATCGCTGCGCGGGCTGCGAGATCTAGGTGGCCGGGCGCGGACCCAAGC
CGTGCCGCCGGCGCCATGAAGGGCAAGGAGGAAAAGGAGGGCGGCGCGCG
GCTGGGCACTGGTGGCGGCGGCACGCCTGATAAGAGCCCGAGTGCGCAAG
AGCTCAAGGAGCAGGGAAACCGGCTCTTCGTGGGCCGCAAGTACCCGGAG
GCGGCGGCCTGCTACGGCCGCGCCATCACTCGGAACCCACTTGTGGCAGT
GTACTACACTAACCGGGCCCTGTGCTATCTGAAGATGCAGCAGCCTGAAC
AGGCACTTGCTGACTGCCGGCGAGCCCTGGAGCTGGACGGGCAGTCTGTG
AAGGCGCACTTCTTCCTGGGGCAGTGCCAGCTGGAGATGGAGAGTTATGA
TGAGGCCATTGCCAATCTGCAGCGAGCCTATAGTTTGGCCAAGGAGCAGC
GACTCAACTTTGGGGATGATATTCCTAGTGCCCTTCGCATTGCTAAGAAG
AAGCGCTGGAACAGTATCGAGGAACGGCGCATCCACCAGGAGAGTGAGCT
GCATTCATATCTCACCAGGCTCATTGCTGCTGAGCGAGAGAGGGAACTGG
AGGAGTGTCAGCGGAACCACGAGGGTCATGAAGATGATGGCCACATCCGG
GCCCAGCAGGCCTGCATTGAGGCCAAGCACGATAAATACATGGCAGATAT
GGATGAGCTCTTCTCTCAGGTGGACGAGAAAAGAAAGAAGCGAGATATCC
CTGACTACTTGTGTGGCAAGATTAGCTTTGAGCTGATGCGGGAACCCTGC
ATTACACCCAGTGGTATCACCTATGACCGCAAGGACATTGAGGAGCACCT
GCAGCGTGTGGGCCACTTTGACCCTGTGACCCGGAGCCCTCTGACCCAGG
AACAGCTCATCCCCAATTTGGCCATGAAGGAAGTCATTGACGCTTTCATC
TCTGAGAACGGCTGGGTAGAGGACTATTGAGGCCCCATGTCCTGCCTGGC
ACCCTGGCCCAGGAGGATCTGGAGACGGAAGCTCCAGTCCCTGTATAGTT
TGTGTCCCTGGGCCTGCCCCCATCGGCCCTGCTGATGGGTTCTGAACTGC
TCCCCTTCTCAGCATACCCCTTGCTGGACCATGAGCCTCCCTTGTCCCCC
TTCTGGGCTGGAGAGTGGGTGAGGGTGGGCTGAGGTTGCTGCTGCTGCCA
CTGTCCTGTAATAAAGTCTGTGACACT
SEQ ID NO: 8 mouse CHIP protein sequence (GenPept
Accession BAB22329)
MKGKEEKEGGARLGTGGGGTPDKSPSAQELKEQGNRLFVGRKYPEAAACY
GRAITRNPLVAVYYTNRALCYLKMQQPEQALADCRRALELDGQSVKAHFF
LGQCQLEMESYDEAIANLQRAYSLAKEQRLNFGDDIPSALRIAKKKRWNS
IEERRIHQESELHSYLTRLIAAERERELEECQRNHEGHEDDGHIRAQQAC
IEAKHDKYMADMDELFSQVDEKRKKRDIPDYLCGKISFELMREPCITPSG
ITYDRKDIEEHLQRVGHFDPVTRSPLTQEQLIPNLAMKEVIDAFISENGW
VEDY
SEQ ID No: 9 Rattus CHIP (GenBank Accession
XM_213270.2 or XM_213270.3)
GGAAGTTCCGGTGGCGGATCGCCGACCGGGCGGAGCTGATAGCTGCGCGG
GCTGCGAGATCTAGGTGGCCGGGCGCGGAGCCCAAGCCGTGCCGCGCGGC
GCCATGAAGGGCAAGGAGGAAAAGGAGGGCGGCGCGCGGCTGGGCACTGG
TGGCGGCGGCAGCCCTGATAAGAGCCCGAGTGCGCAAGAGCTCAAGGAGC
AGGGAAACCGGCTCTTCGTGGGCCGCAAGTACCCGGAGGCGGCGGCCTGC
TACGGCCGCGCCATCACCCGGAACCCACTTGTGGCAGTGTACTACACCAA
CCGGGCCCTGTGCTATCTGAAGATGCAGCAGCCTGAACAGGCACTTGCTG
ACTGCCGGAGAGCCCTGGAGCTGGATGGGCAGTCTGTGAAGGCGCACTTC
TTCCTGGGGCAGTGCCAGCTAGAGATGGAGAGTTATGATGAGGCCATTGC
CAATCTGCAGCGAGCCTATAGTTTGGCCAAGGAGCAGCGACTCAACTTTG
GGGATGATATTCCTAGTGCCCTTCGCATTGCTAAGAAGAAGCGCTGGAAC
AGTATCGAGGAACGGCGCATCCACCAGGAGAGTGAGCTGCACTCCTATCT
CACCAGGCTCATTGCTGCTGAGCGAGAGAGGGAACTGGAAGAGTGTCAGC
GGAACCACGAGGGTGATGAGGATGATGGCCACATCAGGGCCCAGCAGGCC
TGCATTGAGGCCAAGCACGATAAATACATGGCGGACATGAATGAGCTCTT
CTCTCAGGTGGATGAGAAAAGAAAGAAGCGAGATATCCCTGACTACTTGT
GTGGCAAGATCAGCTTTGAGCTGATGCGGGAACCCTGCATTACACCCAGT
GGCATCACCTATGACCGCAAGGACATTGAGGAGCACCTGCAGCGCGTGGG
CCATTTCGATCCTGTGACCCGGAGCCCTCTGACCCAGGAACAGCTCATCC
CCAACTTAGCCATGAAGGAAGTCATTGATGCTTTCATCTCTGAGAATGGC
TGGGTAGAGGACTACTGATACCCCATGTCCTGCCTGGCACCCTGGCCCAG
GAGGGTCTGGAGGCAGAAGCTCCAGTCCCTGTATATAGTTTGTGTCCCTG
GGCCTGCCCCCATCGGTCCTGATGATGGGTTCTGAACTGCTCCCCTTCTC
AGCATATCCCTTGCTGGGCCATGAGCCTCCCTTGTCTCCCTTCTGGGCTG
GAGAGCTGGTGAGGGTGGGTTGCTGCTGCTGCTGCCACTGTCCTGTAATA
AAGTCTGTGAGCACT
SEQ ID NO: 10 Rattus CHIP protein sequence
(GenPept Accession XP_213270)
MKGKEEKEGGARLGTGGGGSPDKSPSAQELKEQGNRLFVGRKYPEAAACY
GRAITRNPLVAVYYTNRALCYLKMQQPEQALADCRRALELDGQSVKAHFF
LGQCQLEMESYDEAIANLQRAYSLAKEQRLNFGDDIPSALRIAKKKRWNS
IEERRIHQESELHSYLTRLIAAERERELEECQRNHEGDEDDGHIRAQQAC
IEAKHDKYMADMNELFSQVDEKRKKRDIPDYLCGKISFELMREPCITPSG
ITYDRKDIEEHLQRVGHFDPVTRSPLTQEQLIPNLAMKEVIDAFISENGW
VEDY
SEQ ID NO: 11 human CHIP cDNA (STUB1) (GenBank
Accession NM_005861.2)
GCTGTTGCGGGAGCGCGCCCTCAGCGAAGCAAGTGAGGCATCTCACTGGG
AAAGTCGAATGTGTGTGGCGGCCGCCGCCGAGGCGGGTTCCGAAGAGACC
TCAGCAGGGCAGGCCAGGGCCTACGCGAACGCCCACCCTTAAGAGCGCGG
GGACAGGGAACTGGAGCGTTCCTCCCAGCCCCCGACGTCGCGGGCCCAGT
GTCCCCGTCCAGGCTGGTTGGGCGCACGCGCGGCCCCACTCGCCCCCACG
CGTGCGTCCCCGCTGGTCCCGCCCCCGGCCGGAAGTTCCGGCGGCGGAGC
TGGGCCGGGCCCGAGCGGATCGCGGGCTCGGGCTGCGGGGCTCCGGCTGC
GGGCGCTGGGCCGCGAGGCGCGGAGCTTGGGAGCGGAGCCCAGGCCGTGC
CGCGCGGCGCCATGAAGGGCAAGGAGGAGAAGGAGGGCGGCGCACGGCTG
GGCGCTGGCGGCGGAAGCCCCGAGAAGAGCCCGAGCGCGCAGGAGCTCAA
GGAGCAGGGCAATCGTCTGTTCGTGGGCCGAAAGTACCCGGAGGCGGCGG
CCTGCTACGGCCGCGCGATCACCCGGAACCCGCTGGTGGCCGTGTATTAC
ACCAACCGGGCCTTGTGCTACCTGAAGATGCAGCAGCACGAGCAGGCCCT
GGCCGACTGCCGGCGCGCCCTGGAGCTGGACGGGCAGTCTGTGAAGGCGC
ACTTCTTCCTGGGGCAGTGCCAGCTGGAGATGGAGAGCTATGATGAGGCC
ATCGCCAATCTGCAGCGAGCTTACAGCCTGGCCAAGGAGCAGCGGCTGAA
CTTCGGGGACGACATCCCCAGCGCTCTTCGAATCGCGAAGAAGAAGCGCT
GGAACAGCATTGAGGAGCGGCGCATCCACCAGGAGAGCGAGCTGCACTCC
TACCTCTCCAGGCTCATTGCCGCGGAGCGTGAGAGGGAGCTGGAAGAGTG
CCAGCGAAACCACGAGGGTGATGAGGACGACAGCCACGTCCGGGCCCAGC
AGGCCTGCATTGAGGCCAAGCACGACAAGTACATGGCGGACATGGACGAG
CTTTTTTCTCAGGTGGATGAGAAGAGGAAGAAGCGAGACATCCCCGACTA
CCTGTGTGGCAAGATCAGCTTTGAGCTGATGCGGGAGCCGTGCATCACGC
CCAGTGGCATCACCTACGACCGCAAGGACATCGAGGAGCACCTGCAGCGT
GTGGGTCATTTTGACCCCGTGACCCGGAGCCCCCTGACCCAGGAACAGCT
CATCCCCAACTTGGCTATGAAGGAGGTTATTGACGCATTCATCTCTGAGA
ATGGCTGGGTGGAGGACTACTGAGGTTCCCTGCCCTACCTGGCGTCCTGG
TCCAGGGGAGCCCTGGGCAGAAGCCCCCGGCCCCTATACATAGTTTATGT
TCCTGGCCACCCCGACCGCTTCCCCCAAGTTCTGCTGTTGGACTCTGGAC
TGTTTCCCCTCTCAGCATCGCTTTTGCTGGGCCGTGATCGTCCCCCTTTG
TGGGCTGGAAAAGCAGGTGAGGGTGGGCTGGGCTGAGGCCATTGCCGCCA
CTATCTGTGTAATAAAATCCGTGAGCACGAGGTGGGACGTGCTGGTGTGT
GAAAAAAAAAAAAAAAAAAAAAAAAAAAAAAAAAAAAAAAAAAAAA
SEQ ID NO: 12 Rattus CHIP (GenBank Accession
XM_213270.3)
GGCCGCAGGCAGCTTCTCCCGATTCCCGAGGAAGCCTCTCGAGGGTGGTC
TGACCTGCGGGCCTCTCTCCTTCCCTCAATTCCGCCGTCCTACCAGCGGT
CTAGACGTTCCCAGGCTGCCGCATGAGCCCGCGTGCGCCCCGGTGGCGCG
CTAGGACCTGGGCCTAGTGACCGGCCCTCGGAGCGCCACTTCTCCAGCAC
GGACCGCTGCCACGTTCGCGTGGCGTGGGCCGGAGGGTGGGGCTTGACTG
CCAATGGGCGGAGCCTTGGTCTGAATATTGAGGGTAGGTCCTAGTTACTG
TAGGGCGGGGCCGGTAGAGAGCGCAGCGGTAGCTTCTCCGGGCTAAGCGA
ACCCGCTCAATCCGCGAGGCTCCGCCTTTGCTCACGTGTCCCCATGCCAG
CCCCGCCCCACACCGGAAGTTCCGGTGGCGGATCGCCGACCGGGCGGAGC
TGATAGCTGCGCGGGCTGCGAGATCTAGGTGGCCGGGCGCGGAGCCCAAG
CCGTGCCGCGCGGCGCCATGAAGGGCAAGGAGGAAAAGGAGGGCGGCGCG
CGGCTGGGCACTGGTGGCGGCGGCAGCCCTGATAAGAGCCCGAGTGCGCA
AGAGCTCAAGGAGCAGGGAAACCGGCTCTTCGTGGGCCGCAAGTACCCGG
AGGCGGCGGCCTGCTACGGCCGCGCCATCACCCGGAACCCACTTGTGGCA
GTGTACTACACCAACCGGGCCCTGTGCTATCTGAAGATGCAGCAGCCTGA
ACAGGCACTTGCTGACTGCCGGAGAGCCCTGGAGCTGGATGGGCAGTCTG
TGAAGGCGCACTTCTTCCTGGGGCAGTGCCAGCTAGAGATGGAGAGTTAT
GATGAGGCCATTGCCAATCTGCAGCGAGCCTATAGTTTGGCCAAGGAGCA
GCGACTCAACTTTGGGGATGATATTCCTAGTGCCCTTCGCATTGCTAAGA
AGAAGCGCTGGAACAGTATCGAGGAACGGCGCATCCACCAGGAGAGTGAG
CTGCACTCCTATCTCACCAGGCTCATTGCTGCTGAGCGAGAGAGGGAACT
GGAAGAGTGTCAGCGGAACCACGAGGGTGATGAGGATGATGGCCACATCA
GGGCCCAGCAGGCCTGCATTGAGGCCAAGCACGATAAATACATGGCGGAC
ATGAATGAGCTCTTCTCTCAGGTGGATGAGAAAAGAAAGAAGCGAGATAT
CCCTGACTACTTGTGTGGCAAGATCAGCTTTGAGCTGATGCGGGAACCCT
GCATTACACCCAGTGGCATCACCTATGACCGCAAGGACATTGAGGAGCAC
CTGCAGCGCGTGGGCCATTTCGATCCTGTGACCCGGAGCCCTCTGACCCA
GGAACAGCTCATCCCCAACTTAGCCATGAAGGAAGTCATTGATGCTTTCA
TCTCTGAGAATGGCTGGGTAGAGGACTACTGATACCCCATGTCCTGCCTG
GCACCCTGGCCCAGGAGGGTCTGGAGGCAGAAGCTCCAGTCCCTGTATAT
AGTTTGTGTCCCTGGGCCTGCCCCCATCGGTCCTGATGATGGGTTCTGAA
CTGCTCCCCTTCTCAGCATATCCCTTGCTGGGCCATGAGCCTCCCTTGTC
TCCCTTCTGGGCTGGAGAGCTGGTGAGGGTGGGTTGCTGCTGCTGCTGCC
ACTGTCCTGTAATAAAGTCTGTGAGCACTACATTGGCATGTGCTGGTGCA
GTGGGCTTGCCAGTCGCTTGTTGGCTAGCCAAGGAAAGTGGATATGAAGA
CACTGGTGTCCAGATTGAGTGTGGCATGCCACCACCGATCAGGAAAGTAC
AGCGCCTGGG

### ADDITIONAL LITERATURE

Alberti, (2002) J Biol Chem 277, 45920-45927.
Ao, (2000) J Cell Biol 148, 375-384.
Aravind, (2000) Curr Biol 10, R132-134.
Ballinger, (1999) Mol Cell Biol 19, 4535-4545.
Barral, (1998) J Cell Biol 143, 1215-1225.
Barral, (1999) Bioessays 21, 813-823.
Barral, (2002) Science 295, 669-671.
Blumenthal, (2002) Nature 417, 851-854.
Brenner, (1974) Genetics 77, 71-94.
Connell, (2001) Nat Cell Biol 3, 93-96.
Cyr, (2002) Trends Biochem Sci 27, 368-375.
Dai, (2003) Embo J 22, 5446-5458.
Demand, (2001) Curr Biol 11, 1569-1577.
DeRenzo, (2003) Nature 424, 685-689.
Epstein, (1974) Nature 250, 579-580.
Epstein, (1974) J Mol Biol 90, 291-300.
Etheridge, (2002) Dev Dyn 224, 457-460.
Hashizume, (2001) J Biol Chem 276, 14537-14540.
Hatakeyama, (2001) J Biol Chem 276, 33111-33120.
Hershko, A., and Ciechanover, A. (1998). The ubiquitin system. Annu Rev Biochem 67, 425-479.
Heschl, (1990) Comp Biochem Physiol B 96, 633-637.
Hochstrasser, (1996) Annu Rev Genet. 30, 405-439.
Hohfeld, (2001) EMBO Rep 2, 885-890.
Hutagalung, (2002) J Cell Sci 115, 3983-3990.
Imai, (2002) Mol Cell 10, 55-67.
James, (1996) Genetics 144, 1425-1436.
Jiang, (2001) J Biol Chem 276, 42938-42944.
Jones, (2002) Genome Biol 3, RESEARCH0002.
Kaneko, (2003) Biochem Biophys Res Commun 300, 297-304.
Koegl, (1999) Cell 96, 635-644.
Lakowski, (2003) Development 130, 2117-2128.
Lorick, (1999) Proc Natl Acad Sci USA 96, 11364-11369.
Mahoney, (2002) Biochem J 361, 587-595.
Mello, (1991) Embo J 10, 3959-3970.
Murata, (2003) Int J Biochem Cell Biol 35, 572-578.
Murata, (2001) EMBO Rep 2, 1133-1138.
Nikolay, (2004) J Biol Chem 279, 2673-2678.
Pickart, (2004) Cell 116, 181-190.
Price, (2002) J Cell Sci 115, 4013-4023.
Srikakulam, (2004) J Cell Sci 117, 641-652.
Venolia, (1999) Cell Motil Cytoskeleton 42, 163-177.
Venolia, (1990) Genetics 126, 345-353.
Waterston, (1988). Muscle. In The Nematode Caenorhabditis Elegans, W. B. Wood, ed. (N.Y., Cold Spring Harbor Laboratory), pp. 281-335.
Wong, (2000) J Cell Sci 113 (Pt 13), 2421-2432.
Xu, (2002) Proc Natl Acad Sci USA 99, 12847-12852.

1. A pharmaceutical composition comprising an inhibitor/negative regulator/antagonist of the mammalian ortholog of Caenorhabditis elegans CHN-1 and/or of the human CHIP (carboxyl-terminus of Hsc70 interacting protein).

2. (canceled)

3. A method for treating, ameliorating and/or preventing a myopathy or a muscular disease in a subject comprising administering an inhibitor/negative regulator/antagonist of the mammalian ortholog of Caenorhabditis elegans CHN-1 and/or of the human CHIP to mammal in need of such a therapy.

4. The pharmaceutical composition of claim 1, wherein said inhibitor/negative regulator/antagonist is selected from the group consisting of small-binding molecules, intracellular-binding receptors, aptamers, intramers, RNAi (double-stranded RNA), siRNA and anti-CHN-1- or anti-CHI P-antisense molecules.

5. The pharmaceutical composition method of claim 4, wherein said intracellular binding receptor is an intracellular antibody or an antibody fragment.

6. The pharmaceutical composition of claim 4, wherein said anti-CHN-1- or anti-CHIP-antisense molecule comprises a nucleic acid molecule which is the complementary strand of a reversed complementary strand of the coding region of CHN-1, CHIP or a CHN-1/CHIP-homologue.

7. The pharmaceutical composition of claim 1, wherein said CHN-1 ortholog or said CHIP ortholog is selected from the group consisting of a CHN-1- or CHIP-molecule encoded by a nucleotide sequence as shown in SEQ ID NO: 1, SEQ ID NO: 3, SEQ ID NO: 5, SEQ ID NO: 7 or SEQ ID NO: 9, 11 or 12.

8. The pharmaceutical composition of claim 4, wherein said siRNA is selected from the group consisting of the siRNA duplex shown in SEQ ID NOs: 13 and 14, 15 and 16, 17 and 18, 19 and 20, 21 and 22 or 23 and 24.

9. The method of claim 3, wherein said myopathy or muscular disease is a muscular dystrophy.

10. The use or method of claim 9, wherein said muscular dystrophy is selected from the group consisting of Duchenne muscular dystrophy, sarco-glycanopathy, limb-girdle dystrophy, facio-scapulo-humeral dystrophy, dystrophia myotonica, muscular dystrophy and dystrophy of the Becker type.

11. A method for screening inhibitors or negative regulators of CHN-1/CHIP activity or expression comprising the steps of
(a) contacting a cell or a non-human organism expressing CHN-1/CHIP or capable of expressing CHN-1/CHIP with a compound to be tested;
(b) determining the status of ubiquitylation or multi-ubiquitylation in said cell or a cell of said non-human organism in the presence of said compound to be tested when compared to a cell not contacted with said compound; and
(c) identifying the compound which inhibits CHN-1/CHIP activity.

12. The method of claim 11, wherein said non-human organism is selected from the group consisting of C. elegans, yeast, zebrafish, drosophila, mouse, rat, guinea pig, dog and cat.

13. A method for the preparation of a pharmaceutical composition comprising the steps of
(a) identifying a compound capable of inhibiting or negatively-regulating CHN-1/CHIP activity by the method of claim 11; and
(b) formulating said compound with a pharmaceutically acceptable carrier.

14. A non-human transgenic animal comprising a double mutation, wherein the first of said mutations comprises a modification in a gene which leads to a phenotype of a muscular disease and where the second of said mutations comprises a mutation in the ortholog of CHN-1/CHIP.

15. The non-human transgenic animal of claim 14, wherein said first mutation is selected from the group consisting of a mutation in the Dystrophin-, Utropin-, α-, β-, γ-, δ-Sarcoglycan-, Laminin-α2-, Dysferlin-, Integrin α5-, Integrin α7-, α-Dystrobrevin-, α-Dystroglycan-, Calpain 3-, Lamin A-, LARGE- and Caveolin 3-gene.

16. The non-human transgenic animal of claim 14, when said animal is a mouse and wherein said ortholog of CHN-1/CHIP is the mouse ortholog.

17. The non-human transgenic animal of claim 14, where said CHN-1/CHIP ortholog comprises a mutation which leads to a non-functional CHN-1/CHIP expression, function or activity.

18. The non-human transgenic animal of claim 17, wherein said CHN-1/CHIP ortholog mutation is a knock-out mutation

19. The method of claim 3, wherein said inhibitor/negative regulator/antagonist is selected from the group consisting of small-binding molecules, intracellular-binding receptors, aptamers, intramers, RNAi (double-stranded RNA), siRNA and anti-CHN-1- or anti-CHI P-antisense molecules.

20. The method of claim 19, wherein said intracellular binding receptor is an intracellular antibody or an antibody fragment.

21. The method of claim 19, wherein said anti-CHN-1- or anti-CHIP-antisense molecule comprises a nucleic acid molecule which is the complementary strand of a reversed complementary strand of the coding region of CHN-1, CHIP or a CHN1/CHIP-homologue.

22. The method of claim 3, wherein said CHN-1 ortholog or said CHIP ortholog is selected from the group consisting of a CHN-1- or CHIP-molecule encoded by a nucleotide sequence as shown in SEQ ID NO: 1, SEQ ID NO: 3, SEQ ID NO: 5, SEQ ID NO: 7 or SEQ ID NO: 9, 11 or 12.

23. The method of claim 19, wherein said siRNA is selected from the group consisting of the siRNA duplex shown in SEQ ID NOs: 13 and 14, 15 and 16, 17 and 18, 19 and 20, 21 and 22 or 23 and 24.
